SEQUENCE LISTING (37 sequences)

SEQ ID NO: 1 - DNA, 1503 bp, synthetic construct
  misc_feature 1..1503: note = Synthetic
  source 1..1503: mol_type = other DNA
atgaccgccatcaagatgaacaccaacggtgagggtgagactcagcacatccttatgatccctttcatggctcagggacacctcagaccatttctcgagcttgctatgttcctctacaagcgtagccacgtgatcatcacactcctcactactcctctcaacgctggattccttagacatctcctccaccaccacagctacagctcatctggaatcaggatcgtcgagctgcctttcaactctactaaccatggactcccaccgggaatcgagaatactgataagcttactctcccgctcgtggtgagcctcttccattctactatttctctcgatccgcacctcagggactacatctctagacatttcagccctgctaggcctcctctctgcgttatccatgatgtttttctcggctgggttgaccaggtggcaaaggatgttggatctactggtgtggtgttcactaccggtggtgcttatggaacctctgcctacgtttcgatctggaacgatctccctcaccagaactactctgacgaccaagaattccctcttccgggattccctgagaaccacaagttcagaagatctcagctccaccgtttcctcaggtacgctgatggatctgatgactggtccaagtacttccagcctcagctcagacagagcatgaagtctttcggatggctctgcaacagcgtggaagagattgagactctcgggttctctatcctccgtaactacactaagctcccgatctggggaatcggacctcttattgcttctcctgtgcagcactccagcagcgataacaattctacaggtgctgagttcgtgcagtggctctctttgaaagagcctgactctgtcctctacatcagcttcggaagccagaacaccatctctccgactcagatgatggaacttgctgctggacttgagtcctctgagaagccattcctttgggttatcagggctcctttcgggttcgacatcaacgaagaaatgaggcctgagtggcttcctgagggattcgaagagaggatgaaggtcaagaagcaggggaagctcgtttacaagcttggacctcagcttgagatcctcaaccacgagtctatcggaggattcctcactcattgcggctggaactctatccttgagtctcttagagaaggggtgccaatgcttggatggcctttggctgctgaacaggcttacaaccttaagtacctcgaggacgagatgggagttgctgttgaacttgctagaggactcgaaggggagatcagcaaagagaaggttaagcgtatcgtcgagatgatcctcgagaggaacgagggatctaaaggctgggagatgaagaacagggctgttgagatggggaagaagctcaaggacgctgtcaacgaggaaaaagagctgaaggggtcaagcgtgaaggctatcgatgatttcctcgacgctgtgatgcaggctaagcttgagccatctcttcagtga

SEQ ID NO: 2 - protein, 500 aa, Mirabilis jalapa
MTAIKMNTNGEGETQHILMIPFMAQGHLRPFLELAMFLYKRSHVIITLLTTPLNAGFLRHLLHHHSYSSSGIRIVELPFNSTNHGLPPGIENTDKLTLPLVVSLFHSTISLDPHLRDYISRHFSPARPPLCVIHDVFLGWVDQVAKDVGSTGVVFTTGGAYGTSAYVSIWNDLPHQNYSDDQEFPLPGFPENHKFRRSQLHRFLRYADGSDDWSKYFQPQLRQSMKSFGWLCNSVEEIETLGFSILRNYTKLPIWGIGPLIASPVQHSSSDNNSTGAEFVQWLSLKEPDSVLYISFGSQNTISPTQMMELAAGLESSEKPFLWVIRAPFGFDINEEMRPEWLPEGFEERMKVKKQGKLVYKLGPQLEILNHESIGGFLTHCGWNSILESLREGVPMLGWPLAAEQAYNLKYLEDEMGVAVELARGLEGEISKEKVKRIVEMILERNEGSKGWEMKNRAVEMGKKLKDAVNEEKELKGSSVKAIDDFLDAVMQAKLEPSLQ

SEQ ID NO: 3 - DNA, 828 bp, synthetic construct
  misc_feature 1..828: note = Synthetic
  source 1..828: mol_type = other DNA
atgaagatgatgaacggcgaggacgccaacgaccagatgatcaaagagagcttcttcatcacccacgggaaccctatcctcactgttgaggatactcatcctctcaggccgttcttcgaaacttggagggaaaagatcttcagcaagaagccgaaggccatcctcatcatctctggacattgggagactgtgaagcctactgtgaacgctgtgcacatcaacgataccatccacgacttcgatgactacccggctgctatgtaccagttcaagtatcctgctcctggtgagcctgagcttgctagaaaggttgaagagatcctcaagaagtccgggttcgagactgctgagactgatcaaaagaggggtcttgatcacggtgcttgggttccacttatgctcatgtatcctgaggctgacatccctgtgtgccagctttctgttcagcctcatctcgatggaacctaccactacaatcttggaagggctcttgctccgctcaagaacgatggtgttctcatcatcggaagcgggtctgctacacatcctcttgatgagactcctcactacttcgatggtgtggctccttgggctgctgcttttgattcttggcttcgtaaggccctcatcaacggtagattcgaagaggtgaacatctacgagagcaaggctcctaactggaagctcgctcatccatttccagagcacttctaccctctccatgttgttcttggagctgctggtgaaaagtggaaggctgagcttatccactcttcttgggatcatggaaccctctgccacgggtcttacaagttcacttctgcttga

SEQ ID NO: 4 - protein, 275 aa, Beta vulgaris
MKMMNGEDANDQMIKESFFITHGNPILTVEDTHPLRPFFETWREKIFSKKPKAILIISGHWETVKPTVNAVHINDTIHDFDDYPAAMYQFKYPAPGEPELARKVEEILKKSGFETAETDQKRGLDHGAWVPLMLMYPEADIPVCQLSVQPHLDGTYHYNLGRALAPLKNDGVLIIGSGSATHPLDETPHYFDGVAPWAAAFDSWLRKALINGRFEEVNIYESKAPNWKLAHPFPEHFYPLHVVLGAAGEKWKAELIHSSWDHGTLCHGSYKFTSA

SEQ ID NO: 5 - DNA, 1494 bp, synthetic construct
  misc_feature 1..1494: note = Synthetic
  source 1..1494: mol_type = other DNA
atggatcatgctaccctcgctatgatcctcgcgatctggttcatcagcttccacttcatcaagctcttgttcagccagcagacgaccaagcttcttcctcctggacctaaacctcttccgatcatcggtaacatcctcgaggttggaaagaagccgcaccgttcttttgctaacctggctaagatccacggaccgctcatttctcttagactcggatctgtgacgaccatcgtggtgtcatctgctgatgtggccaaagagatgttcctcaagaaggatcacccgctcagcaacaggactatccctaattctgtgaccgctggtgaccaccacaagctgactatgtcttggcttcctgtgtctccgaagtggcgaaacttccgtaagattactgctgtgcacctcttgtctccacagagacttgatgcttgccagaccttcagacatgctaaggttcagcagctctacgagtacgttcaagagtgtgctcagaaaggacaggctgtggatattggcaaggctgcctttactaccagcctcaaccttctgtccaagctgttcttcagcgttgagcttgctcaccacaagagccacactagccaagagttcaaagagctgatctggaacatcatggaagatatcgggaagccgaactacgctgactacttccctatcctcggatgcgttgacccttctggaatcagaagaaggctcgcttgctctttcgacaagctgatcgctgttttccaggggatcatctgcgaaagactcgctcctgattcttctaccactaccaccactactaccgacgacgttctcgatgtgcttctccagcttttcaagcagaacgagcttaccatgggcgagatcaaccatctcctcgtggatatcttcgatgctggaaccgataccaccagctctactttcgagtgggtgatgaccgagcttatcagaaaccctgagatgatggaaaaggcccaagaagagatcaagcaggtcctcggaaaggacaagcagatccaagagagcgacatcatcaaccttccatacctccaggccatcatcaaagagactctcagactccatcctccgaccgttttcttgcttcctagaaaggctgataccgacgtcgagctgtacggttacatcgttcctaaggatgctcagatcctcgtgaacctttgggctatcggtagagatcctaacgcttggcagaacgccgacatttttagccctgagagattcatcggatgcgagatcgatgtgaagggtcgtgatttcggacttctcccatttggagctggtagaaggatctgccctggaatgaacctcgctatcagaatgctcacccttatgctcgctactctcctccagttcttcaactggaagctcgagggtgacatctctcctaaggacctcgatatggacgagaagttcggaatcgctctccaaaagaccaagccgcttaagctcatccctattccgaggtactga

SEQ ID NO: 6 - protein, 497 aa, Beta vulgaris
MDHATLAMILAIWFISFHFIKLLFSQQTTKLLPPGPKPLPIIGNILEVGKKPHRSFANLAKIHGPLISLRLGSVTTIVVSSADVAKEMFLKKDHPLSNRTIPNSVTAGDHHKLTMSWLPVSPKWRNFRKITAVHLLSPQRLDACQTFRHAKVQQLYEYVQECAQKGQAVDIGKAAFTTSLNLLSKLFFSVELAHHKSHTSQEFKELIWNIMEDIGKPNYADYFPILGCVDPSGIRRRLACSFDKLIAVFQGIICERLAPDSSTTTTTTTDDVLDVLLQLFKQNELTMGEINHLLVDIFDAGTDTTSSTFEWVMTELIRNPEMMEKAQEEIKQVLGKDKQIQESDIINLPYLQAIIKETLRLHPPTVFLLPRKADTDVELYGYIVPKDAQILVNLWAIGRDPNAWQNADIFSPERFIGCEIDVKGRDFGLLPFGAGRRICPGMNLAIRMLTLMLATLLQFFNWKLEGDISPKDLDMDEKFGIALQKTKPLKLIPIPRY

SEQ ID NO: 7 - DNA, 1500 bp, synthetic construct
  misc_feature 1..1500: note = Synthetic
  source 1..1500: mol_type = other DNA
atggacaacgctactctcgctgtgatcctctctatcctcttcgtgttctaccacatcttcaagtcgttcttcaccaacagcagctctagaaggcttcctcctggacctaaacctgtgcctatcttcggaaacatcttcgaccttggagagaagccgcaccgttctttcgctaacctctctaagatccacggaccgctcatttctctcaagctcggatctgtgacgaccatcgtggttagctctgcttctgttgccgaagagatgttcctcaagaacgatcaggctctcgccaacaggactatccctgattctgttagagctggggaccacgacaagctctcaatgtcttggcttccggtgtctcaaaagtggcgtaacatgcgtaagatcagcgctgtgcagctcctctcaaaccagaaacttgatgcttctcagcctctcaggcaggctaaggttaagcagttgctctcttacgtgcaggtctgctctgagaagatgcagcctgttgatattggcagggctgcctttactacctctctcaaccttctcagcaacaccttcttcagcatcgagctggcttctcacgagtcgtctgcttctcaagagttcaagcagctcatgtggaacatcatggaagagatcggaaggccgaactacgctgacttcttccctatcctcggttacatcgaccctttcggaatcagaagaaggctcgctggatacttcgataagctcatcgatgtgttccaggacatcatccgtgagagacagaagctccgtagctctaactcttctggtgctaagcagaccaacgatatcctcgatactctcctcaagctccacgaggacaacgagctttctatgcctgagatcaaccacctcctcgtcgatatcttcgatgctggaactgataccaccgcttctactcttgaatgggctatggctgagcttgtgaagaaccctgagatgatgaccaaggtgcagatcgagatcgagcaggctcttggaaaggattgcctcgacatccaagagagcgacatctctaagctcccttacctccaggcgatcatcaaagagactctcagactccatcctccgaccgttttcttgcttcctcgtaaggctgataacgacgtcgagctgtatggatacgtggtgcctaagaacgctcaggtgttggttaacctttgggctatcggaagggatccgaaggtttggaagaatcctgaggtgttcagcccagagagattcctcgattgcaacatcgactacaagggccgtgacttcgagcttcttccatttggagctggaagaaggatctgccctggactcactcttgcttacagaatgctcaacctgatgctcgctacactcctccagaactacaactggaagctcgaggatggaatcaaccctaaggatctcgacatggacgagaagttcggtatcactctccagaaggtgaagcctctccaggttatccctgtgccaagaaactga

SEQ ID NO: 8 - protein, 499 aa, Beta vulgaris
MDNATLAVILSILFVFYHIFKSFFTNSSSRRLPPGPKPVPIFGNIFDLGEKPHRSFANLSKIHGPLISLKLGSVTTIVVSSASVAEEMFLKNDQALANRTIPDSVRAGDHDKLSMSWLPVSQKWRNMRKISAVQLLSNQKLDASQPLRQAKVKQLLSYVQVCSEKMQPVDIGRAAFTTSLNLLSNTFFSIELASHESSASQEFKQLMWNIMEEIGRPNYADFFPILGYIDPFGIRRRLAGYFDKLIDVFQDIIRERQKLRSSNSSGAKQTNDILDTLLKLHEDNELSMPEINHLLVDIFDAGTDTTASTLEWAMAELVKNPEMMTKVQIEIEQALGKDCLDIQESDISKLPYLQAIIKETLRLHPPTVFLLPRKADNDVELYGYVVPKNAQVLVNLWAIGRDPKVWKNPEVFSPERFLDCNIDYKGRDFELLPFGAGRRICPGLTLAYRMLNLMLATLLQNYNWKLEDGINPKDLDMDEKFGITLQKVKPLQVIPVPRN

SEQ ID NO: 9 - DNA, 696 bp, synthetic construct
  misc_feature 1..696: note = Synthetic
  source 1..696: mol_type = other DNA
atgagctacagcaagcagggaatcgtgcaagagatgaagaccaagtaccacatggaaggctctgtgaacggacacgagttcaccattgaaggtgtcggaactggatacccgtacgagggaaagcaaatgagcgagctggttatcatcaagccggctggaaagcctcttccgttctctttcgatatcctctcgagcgtgttccagtacggaaacagatgcttcactaagtaccctgctgacatgccggactacttcaagcaagctttccctgacggaatgagctacgagcgatctttcttgttcgaggatggtgctgtggctaccgcttcttggaacattagacttgaggggaactgcttcatccacaagagcatcttccacggggttaacttccctgctgatggaccggtgatgaagaagaaaaccatcgactgggacaagagcttcgagaagatgaccgtgtctaaagaggtgctcagaggcgacgttaccatgttccttatgcttgaaggtggcggttctcacagatgccagttccactctacttacaagaccgagaagcctgtgactctccctcctaaccatgttgttgagcaccagatcgtgaggactgatcttggacaaagcgctaagggattcaccgttaagcttgaggctcatgctgctgctcatgtgaaccctctcaaggtgaagtag

SEQ ID NO: 10 - protein, 231 aa, Acropora millepora
MSYSKQGIVQEMKTKYHMEGSVNGHEFTIEGVGTGYPYEGKQMSELVIIKPAGKPLPFSFDILSSVFQYGNRCFTKYPADMPDYFKQAFPDGMSYERSFLFEDGAVATASWNIRLEGNCFIHKSIFHGVNFPADGPVMKKKTIDWDKSFEKMTVSKEVLRGDVTMFLMLEGGGSHRCQFHSTYKTEKPVTLPPNHVVEHQIVRTDLGQSAKGFTVKLEAHAAAHVNPLKVK

SEQ ID NO: 11 - DNA, 474 bp, synthetic construct
  misc_feature 1..474: note = Synthetic
  source 1..474: mol_type = other DNA
atggacctgaaagtggaattctataatagcaatccgagcgataccaccaatagcattaatccgcagtttaaagttaccaataccggtagcagcgcaattgatctgagcaaactgaccctgcgttattactataccgttgatggtcagaaagatcagacattttggtgtgatcatgcagccattattggtagcaatggtagctataatggcattaccagcaatgtgaaaggcacctttgttaaaatgagcagcagcacaaataacgccgatacctatctggaaattagctttaccggtggcaccctggaaccgggtgcacatgttcagattcagggtcgttttgccaaaaatgattggagcaattatacccagagcaacgactatagctttaaaagcgcaagccagtttgttgaatgggatcaagttaccgcatatctgaatggtgttctggtttggggtaaagaaccgggttaa

SEQ ID NO: 12 - protein, 157 aa, Clostridium cellulovorans
MDLKVEFYNSNPSDTTNSINPQFKVTNTGSSAIDLSKLTLRYYYTVDGQKDQTFWCDHAAIIGSNGSYNGITSNVKGTFVKMSSSTNNADTYLEISFTGGTLEPGAHVQIQGRFAKNDWSNYTQSNDYSFKSASQFVEWDQVTAYLNGVLVWGKEPG

SEQ ID NO: 13 - DNA, 753 bp, synthetic construct
  misc_feature 1..753: note = Synthetic
  source 1..753: mol_type = other DNA
gtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgc

SEQ ID NO: 14 - DNA, 769 bp, synthetic construct
  misc_feature 1..769: note = Synthetic
  source 1..769: mol_type = other DNA
ggtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgataacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggacctcgactctagaggatccc

SEQ ID NO: 15 - DNA, 1084 bp, synthetic construct
  misc_feature 1..1084: note = Synthetic
  source 1..1084: mol_type = other DNA
aaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaacc

SEQ ID NO: 16 - DNA, 666 bp, synthetic construct
  misc_feature 1..666: note = Synthetic
  source 1..666: mol_type = other DNA
atgagcgtgatcgccaagcagatgacttacaaggtgtacatgagcggaaccgtgaacggacattacttcgaggttgaaggtgacggaaagggaaagccttatgagggtgagcagactgtgaagctcactgtgacaaaaggtggacctcttccgttcgcttgggatatcctttctccgcaatgccaatacggcagcatcccgtttactaagtaccctgaggacatcccggattacgtgaagcaatctttcccagagggatacacctgggagcgtatcatgaatttcgaggatggtgctgtgtgcaccgtgagcaacgattcttctatccagggcaactgcttcatctaccacgtgaagttcagcggactcaacttccctcctaacggacctgtgatgcagaaaaagactcaaggatgggagcctaacaccgagagactcttcgctagagatggaatgctcctcgggaacaacttcatggctcttaagcttgaaggcggaggacattacctctgcgagttcaagacaacctacaaggccaagaaaccggtgaagatgccgggataccattacgtggacagaaagctcgatgtgaccaaccacaacaaggactacacctctgttgagcagtgcgagatctctatcgctcgtaagcctgttgtggcttaa

SEQ ID NO: 17 - DNA, 666 bp, synthetic construct
  misc_feature 1..666: note = Synthetic
  source 1..666: mol_type = other DNA
atgagcgtgattgcaaagcagatgacctataaagtttatatgagcggcaccgtgaacggccattattttgaagttgaaggtgatggtaaaggcaaaccgtatgaaggtgaacagaccgttaaactgaccgttaccaaaggtggtccgctgccgtttgcatgggatattctgagtccgcagtgtcagtatggtagcattccgtttaccaaatatccggaagatatcccggattatgtgaaacagagctttccggaaggttatacctgggaacgtattatgaattttgaagatggtgccgtttgtaccgttagcaatgatagcagcattcagggtaattgctttatctaccacgtgaaatttagcggtctgaattttccgcctaatggtccggttatgcagaaaaagacccaaggttgggaaccgaataccgaacgtctgtttgcacgtgatggtatgctgctgggtaataactttatggcactgaaactggaaggtggtggtcattatctgtgtgaattcaaaaccacgtacaaagccaaaaagccggttaaaatgcctggctatcattatgttgatcgcaaactggatgtgaccaaccacaataaagattacaccagcgttgaacagtgcgaaattagcattgcacgtaaaccggttgttgcctaa

SEQ ID NO: 18 - protein, 221 aa, Acropora millepora
MSVIAKQMTYKVYMSGTVNGHYFEVEGDGKGKPYEGEQTVKLTVTKGGPLPFAWDILSPQCQYGSIPFTKYPEDIPDYVKQSFPEGYTWERIMNFEDGAVCTVSNDSSIQGNCFIYHVKFSGLNFPPNGPVMQKKTQGWEPNTERLFARDGMLLGNNFMALKLEGGGHYLCEFKTTYKAKKPVKMPGYHYVDRKLDVTNHNKDYTSVEQCEISIARKPVVA

SEQ ID NO: 19 - DNA, 684 bp, synthetic construct
  misc_feature 1..684: note = Synthetic
  source 1..684: mol_type = other DNA
atgagcgtgatcaagcaggtcatgaagaccaagcttcacctcgagggaactgtgaacggacacgatttcaccatcgaaggcaaaggtgagggaaagccttacgagggacttcagcacatgaagatgactgtgactaagggtgctccgcttcctttctctgtgcatatcctcacaccgagccacatgtatggaagcaagccgttcaacaagtaccctgctgatatcccggattaccacaagcagtctttcccagagggaatgtcttgggagcgtagcatgattttcgaggatggtggtgtgtgcaccgcctctaaccattcttctatcaacctccaagagaactgcttcatctacgacgtgaagttccacggtgtgaaccttccacctgatggacctgtgatgcaaaagactattgctggatgggagccgtctgtcgagactctctatgttagagatgggatgctcaagagcgacaccgctatggtgttcaagcttaaaggtggtggacaccaccgtgtggatttcaagactacctacaaggctaagaagccggtcaagctccctgagttccatttcgttgagcacagactcgagcttaccaagcacgataaggacttcactacctgggatcagcaagaagctgctgagggacatttcagccctttgcctaaggctctcccatag

SEQ ID NO: 20 - DNA, 684 bp, synthetic construct
  misc_feature 1..684: note = Synthetic
  source 1..684: mol_type = other DNA
atgagcgtgattaaacaggtgatgaaaaccaaactgcatctggaaggcaccgttaatggtcatgatttcaccattgaaggtaaaggtgaaggcaaaccgtatgaaggtctgcagcacatgaaaatgaccgttaccaaaggtgcaccgctgccgtttagcgttcatattctgaccccgagccacatgtatggtagcaaaccgtttaacaaatatccggcagatatcccggattatcacaaacagagctttccggaaggtatgagctgggaacgtagcatgatttttgaagatggtggtgtttgtaccgcaagcaatcatagcagcattaatctgcaagaaaactgcttcatctacgacgtgaaattccatggtgttaatctgcctccggatggtccggttatgcagaaaaccattgcaggttgggaaccgagcgttgaaaccctgtatgttcgtgatggtatgctgaaaagcgataccgccatggtttttaaactgaaaggtggtggtcatcatcgtgtggatttcaaaaccacctataaagcaaagaaaccggttaaactgccggaatttcattttgttgaacatcgtctggaactgaccaaacacgataaagattttaccacctgggatcagcaagaagcagcagaaggtcattttagtccgctgccgaaagcactgccgtaa

SEQ ID NO: 21 - protein, 227 aa, Echinopora forskaliana
MSVIKQVMKTKLHLEGTVNGHDFTIEGKGEGKPYEGLQHMKMTVTKGAPLPFSVHILTPSHMYGSKPFNKYPADIPDYHKQSFPEGMSWERSMIFEDGGVCTASNHSSINLQENCFIYDVKFHGVNLPPDGPVMQKTIAGWEPSVETLYVRDGMLKSDTAMVFKLKGGGHHRVDFKTTYKAKKPVKLPEFHFVEHRLELTKHDKDFTTWDQQEAAEGHFSPLPKALP

SEQ ID NO: 22 - DNA, 72 bp, synthetic construct
  misc_feature 1..72: note = Synthetic
  source 1..72: mol_type = other DNA
atggcgaacaagcacctgagccttagcctcttcctcgtgctcctgggcctctccgcctccctcgcctccggc

SEQ ID NO: 23 - DNA, 72 bp, synthetic construct
  misc_feature 1..72: note = Synthetic
  source 1..72: mol_type = other DNA
atggccaacaagcacctcagcttgtctttgttccttgtgctccttgggctctctgcttctcttgcttctgga

SEQ ID NO: 24 - protein, 24 aa, synthetic construct
  REGION 1..24: note = Synthetic
MANKHLSLSLFLVLLGLSASLASG

SEQ ID NO: 25 - DNA, 69 bp, synthetic construct
  misc_feature 1..69: note = Synthetic
  source 1..69: mol_type = other DNA
atggagttttccatgatttttatgattagcttctctgtattgattttgtgctcctcactggcatatggt

SEQ ID NO: 26 - protein, 23 aa, synthetic construct
  REGION 1..23: note = Synthetic
MEFSMIFMISFSVLILCSSLAYG

SEQ ID NO: 27 - DNA, 75 bp, synthetic construct
  misc_feature 1..75: note = Synthetic
  source 1..75: mol_type = other DNA
atgaggcaacaatatgtcttcactactctcacgttgctcatcctgttttccctcagttgttcaacaacattagcc

SEQ ID NO: 28 - protein, 25 aa, synthetic construct
  REGION 1..25: note = Synthetic
MRQQYVFTTLTLLILFSLSCSTTLA

SEQ ID NO: 29 - DNA, 515 bp, synthetic construct
  misc_feature 1..515: note = Synthetic
  source 1..515: mol_type = other DNA
ccacccctaaaacacccatatctaatcctatatatggttttcaaatattttcttttaatcatggttagtttttttgttaattttctatacctactttttaccttaaacttcacatccatttaatcaccaacaattggtataaccccacacgttgttttctttctgtttatatcttacaacttacaaccaaaacatccacactacacacacatatatatattcattccaatctgctaaattggtatccaaaactactcaactttctctctgaattcctccaagttaggttagtgttttccatttgcatttttgtgttaaaagttgtttctcttgagagttctaaatgcatgtctatgttttgaaatggggtttactttttgtcttttgttcataatgtaaatttattggatattttgctgtttatctgcaatgtaattgccaatgatttgattcttgtaggagtttggagagtggtcaccgctgaagcaaagccaaatttcttgggggaaaagaaa

SEQ ID NO: 30 - DNA, 1745 bp, synthetic construct
  misc_feature 1..1745: note = Synthetic
  source 1..1745: mol_type = other DNA
gttatataacaattttagtaacaaaagacctgattgatataacattaaatcatcaataatttaagatgtaattagaatgttttaaattttagggtcaatttaaaatgagagtcataatttgaggatgtctaatgaaattaacattgttctttaaattaaatttaatataatttatagaaataaattttattttggtagaatttagaactctaagataatttcccatttaacccattaagaaaattttagatttttgttatagaaggtgaagcagggaggtgtttaggtttttcaagattaaagttattataatttcacctgtgatacagttgaaaaatcaattttcatattttggttgtacatattctgtagaatgtagtaacataataatagcttttaaataaatggacaataattgccttaccgttaataacatgggacagaagaaaaaaagtgtttttgcctttgttgaatctaatgaatccaagaaaagaaacgagggttaaagaagaaggaagcagggatcatcagcttggatcgatctgctcatggaaattaaagccccacccacagggagccatcagcctcgttgacattagctggaaatagggcccaatttaaccgccatcgctacggaaacttatttcctaagttagccaagcattcgcaatcacaaaccgaggcaatcattaattctttttaatggtgttggatggttatattatatctcgattatatttttttttttaaaaagcgaagttgaatgtctaaataggaagtaatttttttaatattatttttttataatatttgaatccgatatcttatttaaaaaccatcgaaatttttattactcaatcattaccgaaatagaattgggctaaaatatttcgaaaactaaaagtttcactgtttatattgaaaaacgaggctttgtgattcttataaatttaattcattgaaatttcatcaagtaaaacggaagaattataaatctctaaaatgatagataaaaatgtcacaaataaagccattgtgacacccattaaaggagtctttccccatccaggggctaccttaccatattccgaattcaatggtttcaaaaaatctcctacaacaattcgtcttggactagatctagaactactattaacgttttgtgtagccataaatcttattttgtattcattgagatttgttaactttgtatatcatttcactataaataaacaatcttatcatagacccattgtgatcatgaaattatataataatggaaacaaaaaccttagttgcaatctctatatctgatttacttgtaagttttactaggtacgccttatatactgcttttgggtgactctctcaacaattaagagatccattcgaggaatacgggaaataattgaagaaacgagcctccccatatgtatgtctatttggtcacatatccactccaaaatctactctgttttccctgcttttccaagcaatgtagttgctcatcaatttctttcctttcaaataaacatcccaacagagtttgctaaagttgataagtatgtcttctagttaactaaagtagcatattttacctaacttcaccctccaatatcctaaataaaaagctcccatttcttatccaatcaaaacatccataaatttttgttcaaggaccacttcttcccttccattttactttgttttagttgccataacgtcaccttccaatacaacccaca

SEQ ID NO: 31 - DNA, 7782 bp, synthetic construct
  misc_feature 1..7782: note = Synthetic
  source 1..7782: mol_type = other DNA
aaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccaatgaagatgatgaacggcgaggacgccaacgaccagatgatcaaagagagcttcttcatcacccacgggaaccctatcctcactgttgaggatactcatcctctcaggccgttcttcgaaacttggagggaaaagatcttcagcaagaagccgaaggccatcctcatcatctctggacattgggagactgtgaagcctactgtgaacgctgtgcacatcaacgataccatccacgacttcgatgactacccggctgctatgtaccagttcaagtatcctgctcctggtgagcctgagcttgctagaaaggttgaagagatcctcaagaagtccgggttcgagactgctgagactgatcaaaagaggggtcttgatcacggtgcttgggttccacttatgctcatgtatcctgaggctgacatccctgtgtgccagctttctgttcagcctcatctcgatggaacctaccactacaatcttggaagggctcttgctccgctcaagaacgatggtgttctcatcatcggaagcgggtctgctacacatcctcttgatgagactcctcactacttcgatggtgtggctccttgggctgctgcttttgattcttggcttcgtaaggccctcatcaacggtagattcgaagaggtgaacatctacgagagcaaggctcctaactggaagctcgctcatccatttccagagcacttctaccctctccatgttgttcttggagctgctggtgaaaagtggaaggctgagcttatccactcttcttgggatcatggaaccctctgccacgggtcttacaagttcacttctgcttgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggagaagcttaaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccaatggatcatgctaccctcgctatgatcctcgcgatctggttcatcagcttccacttcatcaagctcttgttcagccagcagacgaccaagcttcttcctcctggacctaaacctcttccgatcatcggtaacatcctcgaggttggaaagaagccgcaccgttcttttgctaacctggctaagatccacggaccgctcatttctcttagactcggatctgtgacgaccatcgtggtgtcatctgctgatgtggccaaagagatgttcctcaagaaggatcacccgctcagcaacaggactatccctaattctgtgaccgctggtgaccaccacaagctgactatgtcttggcttcctgtgtctccgaagtggcgaaacttccgtaagattactgctgtgcacctcttgtctccacagagacttgatgcttgccagaccttcagacatgctaaggttcagcagctctacgagtacgttcaagagtgtgctcagaaaggacaggctgtggatattggcaaggctgcctttactaccagcctcaaccttctgtccaagctgttcttcagcgttgagcttgctcaccacaagagccacactagccaagagttcaaagagctgatctggaacatcatggaagatatcgggaagccgaactacgctgactacttccctatcctcggatgcgttgacccttctggaatcagaagaaggctcgcttgctctttcgacaagctgatcgctgttttccaggggatcatctgcgaaagactcgctcctgattcttctaccactaccaccactactaccgacgacgttctcgatgtgcttctccagcttttcaagcagaacgagcttaccatgggcgagatcaaccatctcctcgtggatatcttcgatgctggaaccgataccaccagctctactttcgagtgggtgatgaccgagcttatcagaaaccctgagatgatggaaaaggcccaagaagagatcaagcaggtcctcggaaaggacaagcagatccaagagagcgacatcatcaaccttccatacctccaggccatcatcaaagagactctcagactccatcctccgaccgttttcttgcttcctagaaaggctgataccgacgtcgagctgtacggttacatcgttcctaaggatgctcagatcctcgtgaacctttgggctatcggtagagatcctaacgcttggcagaacgccgacatttttagccctgagagattcatcggatgcgagatcgatgtgaagggtcgtgatttcggacttctcccatttggagctggtagaaggatctgccctggaatgaacctcgctatcagaatgctcacccttatgctcgctactctcctccagttcttcaactggaagctcgagggtgacatctctcctaaggacctcgatatggacgagaagttcggaatcgctctccaaaagaccaagccgcttaagctcatccctattccgaggtactgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggagaagcttaaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccaatgaccgccatcaagatgaacaccaacggtgagggtgagactcagcacatccttatgatccctttcatggctcagggacacctcagaccatttctcgagcttgctatgttcctctacaagcgtagccacgtgatcatcacactcctcactactcctctcaacgctggattccttagacatctcctccaccaccacagctacagctcatctggaatcaggatcgtcgagctgcctttcaactctactaaccatggactcccaccgggaatcgagaatactgataagcttactctcccgctcgtggtgagcctcttccattctactatttctctcgatccgcacctcagggactacatctctagacatttcagccctgctaggcctcctctctgcgttatccatgatgtttttctcggctgggttgaccaggtggcaaaggatgttggatctactggtgtggtgttcactaccggtggtgcttatggaacctctgcctacgtttcgatctggaacgatctccctcaccagaactactctgacgaccaagaattccctcttccgggattccctgagaaccacaagttcagaagatctcagctccaccgtttcctcaggtacgctgatggatctgatgactggtccaagtacttccagcctcagctcagacagagcatgaagtctttcggatggctctgcaacagcgtggaagagattgagactctcgggttctctatcctccgtaactacactaagctcccgatctggggaatcggacctcttattgcttctcctgtgcagcactccagcagcgataacaattctacaggtgctgagttcgtgcagtggctctctttgaaagagcctgactctgtcctctacatcagcttcggaagccagaacaccatctctccgactcagatgatggaacttgctgctggacttgagtcctctgagaagccattcctttgggttatcagggctcctttcgggttcgacatcaacgaagaaatgaggcctgagtggcttcctgagggattcgaagagaggatgaaggtcaagaagcaggggaagctcgtttacaagcttggacctcagcttgagatcctcaaccacgagtctatcggaggattcctcactcattgcggctggaactctatccttgagtctcttagagaaggggtgccaatgcttggatggcctttggctgctgaacaggcttacaaccttaagtacctcgaggacgagatgggagttgctgttgaacttgctagaggactcgaaggggagatcagcaaagagaaggttaagcgtatcgtcgagatgatcctcgagaggaacgagggatctaaaggctgggagatgaagaacagggctgttgagatggggaagaagctcaaggacgctgtcaacgaggaaaaagagctgaaggggtcaagcgtgaaggctatcgatgatttcctcgacgctgtgatgcaggctaagcttgagccatctcttcagtgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagat

SEQ ID NO: 32 - DNA, 3957 bp, synthetic construct
  misc_feature 1..3957: note = Synthetic
  source 1..3957: mol_type = other DNA
atggatcatgctaccctcgctatgatcctcgcgatctggttcatcagcttccacttcatcaagctcttgttcagccagcagacgaccaagcttcttcctcctggacctaaacctcttccgatcatcggtaacatcctcgaggttggaaagaagccgcaccgttcttttgctaacctggctaagatccacggaccgctcatttctcttagactcggatctgtgacgaccatcgtggtgtcatctgctgatgtggccaaagagatgttcctcaagaaggatcacccgctcagcaacaggactatccctaattctgtgaccgctggtgaccaccacaagctgactatgtcttggcttcctgtgtctccgaagtggcgaaacttccgtaagattactgctgtgcacctcttgtctccacagagacttgatgcttgccagaccttcagacatgctaaggttcagcagctctacgagtacgttcaagagtgtgctcagaaaggacaggctgtggatattggcaaggctgcctttactaccagcctcaaccttctgtccaagctgttcttcagcgttgagcttgctcaccacaagagccacactagccaagagttcaaagagctgatctggaacatcatggaagatatcgggaagccgaactacgctgactacttccctatcctcggatgcgttgacccttctggaatcagaagaaggctcgcttgctctttcgacaagctgatcgctgttttccaggggatcatctgcgaaagactcgctcctgattcttctaccactaccaccactactaccgacgacgttctcgatgtgcttctccagcttttcaagcagaacgagcttaccatgggcgagatcaaccatctcctcgtggatatcttcgatgctggaaccgataccaccagctctactttcgagtgggtgatgaccgagcttatcagaaaccctgagatgatggaaaaggcccaagaagagatcaagcaggtcctcggaaaggacaagcagatccaagagagcgacatcatcaaccttccatacctccaggccatcatcaaagagactctcagactccatcctccgaccgttttcttgcttcctagaaaggctgataccgacgtcgagctgtacggttacatcgttcctaaggatgctcagatcctcgtgaacctttgggctatcggtagagatcctaacgcttggcagaacgccgacatttttagccctgagagattcatcggatgcgagatcgatgtgaagggtcgtgatttcggacttctcccatttggagctggtagaaggatctgccctggaatgaacctcgctatcagaatgctcacccttatgctcgctactctcctccagttcttcaactggaagctcgagggtgacatctctcctaaggacctcgatatggacgagaagttcggaatcgctctccaaaagaccaagccgcttaagctcatccctattccgaggtactgaggaagcggagctactaacttcagcctgctgaagcaggctggagacgtggaggagaaccctggacctatgaagatgatgaacggcgaggacgccaacgaccagatgatcaaagagagcttcttcatcacccacgggaaccctatcctcactgttgaggatactcatcctctcaggccgttcttcgaaacttggagggaaaagatcttcagcaagaagccgaaggccatcctcatcatctctggacattgggagactgtgaagcctactgtgaacgctgtgcacatcaacgataccatccacgacttcgatgactacccggctgctatgtaccagttcaagtatcctgctcctggtgagcctgagcttgctagaaaggttgaagagatcctcaagaagtccgggttcgagactgctgagactgatcaaaagaggggtcttgatcacggtgcttgggttccacttatgctcatgtatcctgaggctgacatccctgtgtgccagctttctgttcagcctcatctcgatggaacctaccactacaatcttggaagggctcttgctccgctcaagaacgatggtgttctcatcatcggaagcgggtctgctacacatcctcttgatgagactcctcactacttcgatggtgtggctccttgggctgctgcttttgattcttggcttcgtaaggccctcatcaacggtagattcgaagaggtgaacatctacgagagcaaggctcctaactggaagctcgctcatccatttccagagcacttctaccctctccatgttgttcttggagctgctggtgaaaagtggaaggctgagcttatccactcttcttgggatcatggaaccctctgccacgggtcttacaagttcacttctgcttgaggttcaggtgccactaatttttctctcttgaaacaggccggtgacgttgaagagaacccaggtccaatgaccgccatcaagatgaacaccaacggtgagggtgagactcagcacatccttatgatccctttcatggctcagggacacctcagaccatttctcgagcttgctatgttcctctacaagcgtagccacgtgatcatcacactcctcactactcctctcaacgctggattccttagacatctcctccaccaccacagctacagctcatctggaatcaggatcgtcgagctgcctttcaactctactaaccatggactcccaccgggaatcgagaatactgataagcttactctcccgctcgtggtgagcctcttccattctactatttctctcgatccgcacctcagggactacatctctagacatttcagccctgctaggcctcctctctgcgttatccatgatgtttttctcggctgggttgaccaggtggcaaaggatgttggatctactggtgtggtgttcactaccggtggtgcttatggaacctctgcctacgtttcgatctggaacgatctccctcaccagaactactctgacgaccaagaattccctcttccgggattccctgagaaccacaagttcagaagatctcagctccaccgtttcctcaggtacgctgatggatctgatgactggtccaagtacttccagcctcagctcagacagagcatgaagtctttcggatggctctgcaacagcgtggaagagattgagactctcgggttctctatcctccgtaactacactaagctcccgatctggggaatcggacctcttattgcttctcctgtgcagcactccagcagcgataacaattctacaggtgctgagttcgtgcagtggctctctttgaaagagcctgactctgtcctctacatcagcttcggaagccagaacaccatctctccgactcagatgatggaacttgctgctggacttgagtcctctgagaagccattcctttgggttatcagggctcctttcgggttcgacatcaacgaagaaatgaggcctgagtggcttcctgagggattcgaagagaggatgaaggtcaagaagcaggggaagctcgtttacaagcttggacctcagcttgagatcctcaaccacgagtctatcggaggattcctcactcattgcggctggaactctatccttgagtctcttagagaaggggtgccaatgcttggatggcctttggctgctgaacaggcttacaaccttaagtacctcgaggacgagatgggagttgctgttgaacttgctagaggactcgaaggggagatcagcaaagagaaggttaagcgtatcgtcgagatgatcctcgagaggaacgagggatctaaaggctgggagatgaagaacagggctgttgagatggggaagaagctcaaggacgctgtcaacgaggaaaaagagctgaaggggtcaagcgtgaaggctatcgatgatttcctcgacgctgtgatgcaggctaagcttgagccatctcttcagtga

SEQ ID NO: 33 - DNA, 13756 bp, synthetic construct
  source 1..13756: mol_type = other DNA
gtgccgaattcggatccggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgaagatgatgaacggcgaggacgccaacgaccagatgatcaaagagagcttcttcatcacccacgggaaccctatcctcactgttgaggatactcatcctctcaggccgttcttcgaaacttggagggaaaagatcttcagcaagaagccgaaggccatcctcatcatctctggacattgggagactgtgaagcctactgtgaacgctgtgcacatcaacgataccatccacgacttcgatgactacccggctgctatgtaccagttcaagtatcctgctcctggtgagcctgagcttgctagaaaggttgaagagatcctcaagaagtccgggttcgagactgctgagactgatcaaaagaggggtcttgatcacggtgcttgggttccacttatgctcatgtatcctgaggctgacatccctgtgtgccagctttctgttcagcctcatctcgatggaacctaccactacaatcttggaagggctcttgctccgctcaagaacgatggtgttctcatcatcggaagcgggtctgctacacatcctcttgatgagactcctcactacttcgatggtgtggctccttgggctgctgcttttgattcttggcttcgtaaggccctcatcaacggtagattcgaagaggtgaacatctacgagagcaaggctcctaactggaagctcgctcatccatttccagagcacttctaccctctccatgttgttcttggagctgctggtgaaaagtggaaggctgagcttatccactcttcttgggatcatggaaccctctgccacgggtcttacaagttcacttctgcttgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggatcatgctaccctcgctatgatcctcgcgatctggttcatcagcttccacttcatcaagctcttgttcagccagcagacgaccaagcttcttcctcctggacctaaacctcttccgatcatcggtaacatcctcgaggttggaaagaagccgcaccgttcttttgctaacctggctaagatccacggaccgctcatttctcttagactcggatctgtgacgaccatcgtggtgtcatctgctgatgtggccaaagagatgttcctcaagaaggatcacccgctcagcaacaggactatccctaattctgtgaccgctggtgaccaccacaagctgactatgtcttggcttcctgtgtctccgaagtggcgaaacttccgtaagattactgctgtgcacctcttgtctccacagagacttgatgcttgccagaccttcagacatgctaaggttcagcagctctacgagtacgttcaagagtgtgctcagaaaggacaggctgtggatattggcaaggctgcctttactaccagcctcaaccttctgtccaagctgttcttcagcgttgagcttgctcaccacaagagccacactagccaagagttcaaagagctgatctggaacatcatggaagatatcgggaagccgaactacgctgactacttccctatcctcggatgcgttgacccttctggaatcagaagaaggctcgcttgctctttcgacaagctgatcgctgttttccaggggatcatctgcgaaagactcgctcctgattcttctaccactaccaccactactaccgacgacgttctcgatgtgcttctccagcttttcaagcagaacgagcttaccatgggcgagatcaaccatctcctcgtggatatcttcgatgctggaaccgataccaccagctctactttcgagtgggtgatgaccgagcttatcagaaaccctgagatgatggaaaaggcccaagaagagatcaagcaggtcctcggaaaggacaagcagatccaagagagcgacatcatcaaccttccatacctccaggccatcatcaaagagactctcagactccatcctccgaccgttttcttgcttcctagaaaggctgataccgacgtcgagctgtacggttacatcgttcctaaggatgctcagatcctcgtgaacctttgggctatcggtagagatcctaacgcttggcagaacgccgacatttttagccctgagagattcatcggatgcgagatcgatgtgaagggtcgtgatttcggacttctcccatttggagctggtagaaggatctgccctggaatgaacctcgctatcagaatgctcacccttatgctcgctactctcctccagttcttcaactggaagctcgagggtgacatctctcctaaggacctcgatatggacgagaagttcggaatcgctctccaaaagaccaagccgcttaagctcatccctattccgaggtactgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgaccgccatcaagatgaacaccaacggtgagggtgagactcagcacatccttatgatccctttcatggctcagggacacctcagaccatttctcgagcttgctatgttcctctacaagcgtagccacgtgatcatcacactcctcactactcctctcaacgctggattccttagacatctcctccaccaccacagctacagctcatctggaatcaggatcgtcgagctgcctttcaactctactaaccatggactcccaccgggaatcgagaatactgataagcttactctcccgctcgtggtgagcctcttccattctactatttctctcgatccgcacctcagggactacatctctagacatttcagccctgctaggcctcctctctgcgttatccatgatgtttttctcggctgggttgaccaggtggcaaaggatgttggatctactggtgtggtgttcactaccggtggtgcttatggaacctctgcctacgtttcgatctggaacgatctccctcaccagaactactctgacgaccaagaattccctcttccgggattccctgagaaccacaagttcagaagatctcagctccaccgtttcctcaggtacgctgatggatctgatgactggtccaagtacttccagcctcagctcagacagagcatgaagtctttcggatggctctgcaacagcgtggaagagattgagactctcgggttctctatcctccgtaactacactaagctcccgatctggggaatcggacctcttattgcttctcctgtgcagcactccagcagcgataacaattctacaggtgctgagttcgtgcagtggctctctttgaaagagcctgactctgtcctctacatcagcttcggaagccagaacaccatctctccgactcagatgatggaacttgctgctggacttgagtcctctgagaagccattcctttgggttatcagggctcctttcgggttcgacatcaacgaagaaatgaggcctgagtggcttcctgagggattcgaagagaggatgaaggtcaagaagcaggggaagctcgtttacaagcttggacctcagcttgagatcctcaaccacgagtctatcggaggattcctcactcattgcggctggaactctatccttgagtctcttagagaaggggtgccaatgcttggatggcctttggctgctgaacaggcttacaaccttaagtacctcgaggacgagatgggagttgctgttgaacttgctagaggactcgaaggggagatcagcaaagagaaggttaagcgtatcgtcgagatgatcctcgagaggaacgagggatctaaaggctgggagatgaagaacagggctgttgagatggggaagaagctcaaggacgctgtcaacgaggaaaaagagctgaaggggtcaagcgtgaaggctatcgatgatttcctcgacgctgtgatgcaggctaagcttgagccatctcttcagtgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaagcttctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaacagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggctccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 34 - DNA, 11187 bp, synthetic construct
  source 1..11187: mol_type = other DNA
gtgccgaattcggatccggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgaagatgatgaacggcgaggacgccaacgaccagatgatcaaagagagcttcttcatcacccacgggaaccctatcctcactgttgaggatactcatcctctcaggccgttcttcgaaacttggagggaaaagatcttcagcaagaagccgaaggccatcctcatcatctctggacattgggagactgtgaagcctactgtgaacgctgtgcacatcaacgataccatccacgacttcgatgactacccggctgctatgtaccagttcaagtatcctgctcctggtgagcctgagcttgctagaaaggttgaagagatcctcaagaagtccgggttcgagactgctgagactgatcaaaagaggggtcttgatcacggtgcttgggttccacttatgctcatgtatcctgaggctgacatccctgtgtgccagctttctgttcagcctcatctcgatggaacctaccactacaatcttggaagggctcttgctccgctcaagaacgatggtgttctcatcatcggaagcgggtctgctacacatcctcttgatgagactcctcactacttcgatggtgtggctccttgggctgctgcttttgattcttggcttcgtaaggccctcatcaacggtagattcgaagaggtgaacatctacgagagcaaggctcctaactggaagctcgctcatccatttccagagcacttctaccctctccatgttgttcttggagctgctggtgaaaagtggaaggctgagcttatccactcttcttgggatcatggaaccctctgccacgggtcttacaagttcacttctgcttgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatggacaacgctactctcgctgtgatcctctctatcctcttcgtgttctaccacatcttcaagtcgttcttcaccaacagcagctctagaaggcttcctcctggacctaaacctgtgcctatcttcggaaacatcttcgaccttggagagaagccgcaccgttctttcgctaacctctctaagatccacggaccgctcatttctctcaagctcggatctgtgacgaccatcgtggttagctctgcttctgttgccgaagagatgttcctcaagaacgatcaggctctcgccaacaggactatccctgattctgttagagctggggaccacgacaagctctcaatgtcttggcttccggtgtctcaaaagtggcgtaacatgcgtaagatcagcgctgtgcagctcctctcaaaccagaaacttgatgcttctcagcctctcaggcaggctaaggttaagcagttgctctcttacgtgcaggtctgctctgagaagatgcagcctgttgatattggcagggctgcctttactacctctctcaaccttctcagcaacaccttcttcagcatcgagctggcttctcacgagtcgtctgcttctcaagagttcaagcagctcatgtggaacatcatggaagagatcggaaggccgaactacgctgacttcttccctatcctcggttacatcgaccctttcggaatcagaagaaggctcgctggatacttcgataagctcatcgatgtgttccaggacatcatccgtgagagacagaagctccgtagctctaactcttctggtgctaagcagaccaacgatatcctcgatactctcctcaagctccacgaggacaacgagctttctatgcctgagatcaaccacctcctcgtcgatatcttcgatgctggaactgataccaccgcttctactcttgaatgggctatggctgagcttgtgaagaaccctgagatgatgaccaaggtgcagatcgagatcgagcaggctcttggaaaggattgcctcgacatccaagagagcgacatctctaagctcccttacctccaggcgatcatcaaagagactctcagactccatcctccgaccgttttcttgcttcctcgtaaggctgataacgacgtcgagctgtatggatacgtggtgcctaagaacgctcaggtgttggttaacctttgggctatcggaagggatccgaaggtttggaagaatcctgaggtgttcagcccagagagattcctcgattgcaacatcgactacaagggccgtgacttcgagcttcttccatttggagctggaagaaggatctgccctggactcactcttgcttacagaatgctcaacctgatgctcgctacactcctccagaactacaactggaagctcgaggatggaatcaaccctaaggatctcgacatggacgagaagttcggtatcactctccagaaggtgaagcctctccaggttatccctgtgccaagaaactgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaagcttctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaacagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggctccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 35 - DNA, 14527 bp, synthetic construct
  source 1..14527: mol_type = other DNA
gtgccgaattcggatccggaggtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgataacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgctcgagtataagagctcatttttacaacaattaccaacaacaacaaacaacaaacaacattacaattacatttacaattatcgatacaatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactccaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgaggacgtactcggatggaagccggtcttgtcgatcaggatgatctggacgaagagcatcaggggctcgcgccagccgaactgttcgccaggctcaaggcgcggatgcccgacggcgaggatctcgtcgtgacccagggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctgcaagaattcaagcttggagaagcttaaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccaatgaagatgatgaacggcgaggacgccaacgaccagatgatcaaagagagcttcttcatcacccacgggaaccctatcctcactgttgaggatactcatcctctcaggccgttcttcgaaacttggagggaaaagatcttcagcaagaagccgaaggccatcctcatcatctctggacattgggagactgtgaagcctactgtgaacgctgtgcacatcaacgataccatccacgacttcgatgactacccggctgctatgtaccagttcaagtatcctgctcctggtgagcctgagcttgctagaaaggttgaagagatcctcaagaagtccgggttcgagactgctgagactgatcaaaagaggggtcttgatcacggtgcttgggttccacttatgctcatgtatcctgaggctgacatccctgtgtgccagctttctgttcagcctcatctcgatggaacctaccactacaatcttggaagggctcttgctccgctcaagaacgatggtgttctcatcatcggaagcgggtctgctacacatcctcttgatgagactcctcactacttcgatggtgtggctccttgggctgctgcttttgattcttggcttcgtaaggccctcatcaacggtagattcgaagaggtgaacatctacgagagcaaggctcctaactggaagctcgctcatccatttccagagcacttctaccctctccatgttgttcttggagctgctggtgaaaagtggaaggctgagcttatccactcttcttgggatcatggaaccctctgccacgggtcttacaagttcacttctgcttgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctactagaattcgagctcggagaagcttaaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccaatggatcatgctaccctcgctatgatcctcgcgatctggttcatcagcttccacttcatcaagctcttgttcagccagcagacgaccaagcttcttcctcctggacctaaacctcttccgatcatcggtaacatcctcgaggttggaaagaagccgcaccgttcttttgctaacctggctaagatccacggaccgctcatttctcttagactcggatctgtgacgaccatcgtggtgtcatctgctgatgtggccaaagagatgttcctcaagaaggatcacccgctcagcaacaggactatccctaattctgtgaccgctggtgaccaccacaagctgactatgtcttggcttcctgtgtctccgaagtggcgaaacttccgtaagattactgctgtgcacctcttgtctccacagagacttgatgcttgccagaccttcagacatgctaaggttcagcagctctacgagtacgttcaagagtgtgctcagaaaggacaggctgtggatattggcaaggctgcctttactaccagcctcaaccttctgtccaagctgttcttcagcgttgagcttgctcaccacaagagccacactagccaagagttcaaagagctgatctggaacatcatggaagatatcgggaagccgaactacgctgactacttccctatcctcggatgcgttgacccttctggaatcagaagaaggctcgcttgctctttcgacaagctgatcgctgttttccaggggatcatctgcgaaagactcgctcctgattcttctaccactaccaccactactaccgacgacgttctcgatgtgcttctccagcttttcaagcagaacgagcttaccatgggcgagatcaaccatctcctcgtggatatcttcgatgctggaaccgataccaccagctctactttcgagtgggtgatgaccgagcttatcagaaaccctgagatgatggaaaaggcccaagaagagatcaagcaggtcctcggaaaggacaagcagatccaagagagcgacatcatcaaccttccatacctccaggccatcatcaaagagactctcagactccatcctccgaccgttttcttgcttcctagaaaggctgataccgacgtcgagctgtacggttacatcgttcctaaggatgctcagatcctcgtgaacctttgggctatcggtagagatcctaacgcttggcagaacgccgacatttttagccctgagagattcatcggatgcgagatcgatgtgaagggtcgtgatttcggacttctcccatttggagctggtagaaggatctgccctggaatgaacctcgctatcagaatgctcacccttatgctcgctactctcctccagttcttcaactggaagctcgagggtgacatctctcctaaggacctcgatatggacgagaagttcggaatcgctctccaaaagaccaagccgcttaagctcatccctattccgaggtactgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctttacgaattcccatggggagaagcttaaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccaatgaccgccatcaagatgaacaccaacggtgagggtgagactcagcacatccttatgatccctttcatggctcagggacacctcagaccatttctcgagcttgctatgttcctctacaagcgtagccacgtgatcatcacactcctcactactcctctcaacgctggattccttagacatctcctccaccaccacagctacagctcatctggaatcaggatcgtcgagctgcctttcaactctactaaccatggactcccaccgggaatcgagaatactgataagcttactctcccgctcgtggtgagcctcttccattctactatttctctcgatccgcacctcagggactacatctctagacatttcagccctgctaggcctcctctctgcgttatccatgatgtttttctcggctgggttgaccaggtggcaaaggatgttggatctactggtgtggtgttcactaccggtggtgcttatggaacctctgcctacgtttcgatctggaacgatctccctcaccagaactactctgacgaccaagaattccctcttccgggattccctgagaaccacaagttcagaagatctcagctccaccgtttcctcaggtacgctgatggatctgatgactggtccaagtacttccagcctcagctcagacagagcatgaagtctttcggatggctctgcaacagcgtggaagagattgagactctcgggttctctatcctccgtaactacactaagctcccgatctggggaatcggacctcttattgcttctcctgtgcagcactccagcagcgataacaattctacaggtgctgagttcgtgcagtggctctctttgaaagagcctgactctgtcctctacatcagcttcggaagccagaacaccatctctccgactcagatgatggaacttgctgctggacttgagtcctctgagaagccattcctttgggttatcagggctcctttcgggttcgacatcaacgaagaaatgaggcctgagtggcttcctgagggattcgaagagaggatgaaggtcaagaagcaggggaagctcgtttacaagcttggacctcagcttgagatcctcaaccacgagtctatcggaggattcctcactcattgcggctggaactctatccttgagtctcttagagaaggggtgccaatgcttggatggcctttggctgctgaacaggcttacaaccttaagtacctcgaggacgagatgggagttgctgttgaacttgctagaggactcgaaggggagatcagcaaagagaaggttaagcgtatcgtcgagatgatcctcgagaggaacgagggatctaaaggctgggagatgaagaacagggctgttgagatggggaagaagctcaaggacgctgtcaacgaggaaaaagagctgaaggggtcaagcgtgaaggctatcgatgatttcctcgacgctgtgatgcaggctaagcttgagccatctcttcagtgagcttctctagctagagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcgctcagagaggatgcacatgtgaccgagggacacgaagtgatccgtttaaactatcagtgtttgacaggatatattggcgggtaaacctaagagaaaagagcgtttattagaataatcggatatttaaaagggcgtgaaaaggtttatccgttcgtccatttgtatgtgccagccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaagcttctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaacagctgcaaaaagcgcctacccttcggtcgctgcgctccctacgccccgccgcttcgcgtcggcctatcgcngccgctggccgctcaaaaatggctggcctacggccaggcaatctaccagggcgcggacaagccgcgccgtcgccactcgaccgccggcgcccacatcaaggctccgagtgcgcggaacccctatttgtttatttttctaaatacattcaaatatgtatccgctcatgagacaataaccctgataaatgcttcaataatattgaaaaaggaagagtatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaggacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgctcagggaggatatcggggaagaacagtatgtcgagctattttttgacttactggggatcaagcctgattgggagaaaataaaatattatattttactggatgaattgttttagctgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgctcggatctgttggaccggacagtagtcatggttgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggttgaacactct

SEQ ID NO: 36 - DNA, 11166 bp, synthetic construct
  source 1..11166: mol_type = other DNA
cgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccgggccccccctcgaggcgcgccaagctatcaaacaagtttgtacaaaaaagcaggcttcatggccaacaagcacctcagcttgtctttgttccttgtgctccttgggctctctgcttctcttgcttctgggtcttacagcaagcagggaatcgtgcaagagatgaagaccaagtaccacatggaaggctctgtgaacggacacgagttcaccattgaaggtgtcggaactggatacccgtacgagggaaagcaaatgagcgagctggttatcatcaagcctgctggaaagcctctgccgttctctttcgatatcctctcgtctgtgttccagtacgggaaccgttgcttcactaagtaccctgctgatatgccggactacttcaagcaggcattccctgacggaatgagctacgagcgatctttcttgttcgaggatggtgctgtggctaccgcttcttggaacattagacttgaggggaactgcttcatccacaagagcatcttccacggggttaacttcccagctgatggaccggtgatgaagaagaaaaccatcgactgggacaagagcttcgagaagatgaccgtgtctaaagaggtgctcagaggcgacgttaccatgttccttatgcttgaaggtggcggttctcacagatgccagttccactctacttacaagaccgagaagcctgtgactctccctcctaaccatgttgttgagcaccagatcgtgaggactgatcttggacaaagcgctaagggattcaccgttaagcttgaggctcatgctgctgctcatgtgaaccctctcaaggtgaagtaggacccagctttcttgtacaaagtggttcgataattccttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactgaatt

SEQ ID NO: 37 - DNA, 11154 bp, synthetic construct
  source 1..11154: mol_type = other DNA
cgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattggctagagcagcttgccaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgataacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagaccttcctctatataaggaagttcatttcatttggagaggacacgctgaaatcaccagtctctctctacaaatctatctctctcgagctttcgcagatcccggggggcaatgagatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggagtttagcgagagcctgacctattgcatctcccgccgtgcacagggtgtcacgttgcaagacctgcctgaaaccgaactgcccgctgttctacaaccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccacgactccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaagaaatagagtagatgccgaccggatctgtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagttttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagagaccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagacgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttaaactgaaggcgggaaacgacaatctgatccaagctcaagctgctctagcattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgccaagcttaaaatcacttatcaatttcaaaaacagaggttagccgaatgctaagagcttaaaaatggcttcttttgtttctttttcttgcaaacggtggagagaagagggaaatgaagattgaccatatctttttttattatgttttaacatataatattaataatttaatcataattatactttggtgaatgtgacagtggggatatacgtaaagtatataacattatactttttgcaagcagttggctggtctacccaagagtgatcaaattttgagctgccttcaatgagccaatttttgcctataatggataaaggcactttgtctagttcaactgctcacagaataatgttaaaatgaaattaaaacaaggtgggctggtcacaccaaaaaaataaaatattaatgtggtgtttggttagtcgattttatattagttccatggcataccgcctggaaaaggaaaattcatgtaaataatatatttataaaaatttatattaaaactaaatgaaattttagttgaaatagttaagttaaaaagagtaaaatttataatttatcataattttatagaaattgagactaaaaaacattaagagaataaattctataacaaagacaatttagtaaaaatgtccttttaggtaattttaagtactcttaaccaaaataaaaaattcaaatcaaattaaccaaataagataatataacatacggaacatcccacttataatcttacatccccgtaatcttattatgaaaagtaatcttatattactcgaatcaaatgctctcacaaactattatctaaataaagaaaaacacttaatttttataacattttttttcatatatttgaaagattatattttgtatttttacgtaaaaatatttgacatagattgagcacctttttaacataattccaccataagtcaattatgtagatgagaaattggtacaaacaacgtggagccaaatcccaccaaaccatctctcatcctctcctataaaaggcttgctacacatagacaacaatccacacaaaaatacacttaaaattcttttctttctatttggttaaccggtaccgggccccccctcgaggcgcgccaagctatcaaacaagtttgtacaaaaaagcaggcttcatggccaacaagcacctcagcttgtctttgttccttgtgctccttgggctctctgcttctcttgcttctggatctgtgatcaagcaggtcatgaagaccaagctccatctcgagggaactgtgaacggacacgatttcaccatcgaaggcaaaggtgagggaaagccttacgagggacttcagcacatgaagatgactgtgactaagggtgctccgcttcctttctctgtgcatatcctcacaccgagccacatgtatggaagcaagccgttcaacaagtaccctgctgatatcccggattaccacaagcagtctttcccagagggaatgtcttgggagcgtagcatgattttcgaggatggtggtgtgtgcaccgcctctaaccattcttctatcaacctccaagagaactgcttcatctacgacgtgaagttccacggtgtgaaccttccacctgatggacctgtgatgcaaaagactattgctggatgggagccgtctgtcgagactctctatgttagagatgggatgctcaagagcgacaccgctatggtgttcaagcttaaaggtggtggacaccaccgtgtggatttcaagactacctacaaggctaagaagccggtcaagctccctgagttccatttcgttgagcacagactcgagcttaccaagcacgataaggacttcactacctgggatcagcaagaagctgctgagggacatttcagccctttgcctaaggctctcccataggacccagctttcttgtacaaagtggttcgataattccttaattaactagttctagagcggccgcccaccgcggtggagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactgaatt

(19) United States
(12) Patent Application Publication
(10) Pub. No.: US 2025/0075225 A1
(43) Pub. Date: Mar. 6, 2025

(54) Altering the Optical Properties of Fibres

(71) Applicant: Commonwealth Scientific and Industrial Research Organisation, Acton, Australian Capital Territory (AU)

(72) Inventors: Filomena PETTOLINO, Acton (AU); Colleen MACMILLAN, Acton (AU); Vivien ROLLAND, Acton (AU); Xiaoqing LI, Acton (AU); Madeline MITCHELL, Acton (AU)

(73) Assignee: Commonwealth Scientific and Industrial Research Organisation, Acton, Australian Capital Territory (AU)

(21) Appl. No.: 18/724,529

(22) PCT Filed: May 23, 2023
(86) PCT No.: PCT/AU2023/050436

§ 371 Date: Jun. 26, 2024

(30) Foreign Application Priority Data
May 23, 2022 (AU) 2022901373

(51) Int. Cl. C12N 15/82; C07K 14/415; C07K 14/435; C12N 9/02; C12N 9/10; C12N 15/52; D01G 99/00; D02G 3/02; D02G 3/04

(52) U.S. Cl. CPC C12N 15/825; C07K 14/415; C07K 14/43595; C12N 9/0069; C12N 9/0071; C12N 9/1051; C12N 15/52; C12N 15/8223; C12Y 113/11029; D01G 99/005; D02G 3/02; D02G 3/04; D02G 3/042; D10B 2201/02; D10B 2401/20

(57) ABSTRACT

The present disclosure relates generally to nucleic acid constructs comprising a nucleotide sequence comprising one or more pigment producing genes, wherein expression of the nucleotide sequence is under the control of a cotton fibre-specific promoter, and wherein the cotton fibre-specific promoter is capable of driving expression of the nucleotide sequence during two or more fibre development stages, and their use in the production of colour cotton fibre, or for the introduction of one or more pigment producing genes into a cotton plant. The present disclosure further contemplates cotton plants modified to produce cotton fibre, cotton seed, cotton lit, yarn, textiles and associated methods.

### RELATED APPLICATIONS

The application claims priority from Australian Provisional Patent Application No. 2022901373 filed on 23 May 2022, the entire content of which is hereby incorporated by reference.

### FIELD

The present disclosure relates generally to colored plant fibre comprising exogenous pigments produced in planta by the expression of a nucleic acid construct encoding one or more pigment producing genes. The present disclosure further relates to lint, yarn and textiles prepared therefrom, and associated methods for producing plants, lint, yarn and textiles.

### BACKGROUND

Natural plant fibres such as cotton, flax, hemp, jute, sisal, banana, coir, bamboo, and the like have been used for thousands of years for a variety of purposes, e.g., building materials, cosmetics, and textiles. While the use of natural fibres in the textile industry has been partially replaced by the use of synthetic fibres, interest in the use of natural fibres is growing as a more sustainable and environmentally friendly alternative to synthetic fibres. However, the industrial processing of natural fibres, such as cotton, to produce textiles typically involves textile wet-processing, which imparts aesthetic and functional appeal to the textile fabrics and down-stream products. However, textile wet-processing is recognized to be one of the most polluting industrial processes, emitting significant volumes of greenhouse gases and contributing to around 20% of global waste water pollution (see, e.g., Mehra et al., 2021, Toxicology International, 28(2): 165-176).

Textile wet-processing is a multi-stage manufacturing process, including pre-processing (e.g., scouring), processing (e.g., dying and printing) and post-processing (e.g., finishing). While each of these stages consume a significant amount of water, dyes, chemicals and energy, the pre-processing and processing stages present particular issues due to their reliance on harsh chemicals, high temperatures and large volumes of water to process the fabric. For instance, the pre-processing stage involves the scouring of raw cotton fabrics to remove natural hydrophobic impurities (e.g., fats, waxes, pectins and proteins) using a heated alkaline solution, such as sodium hydroxide. Thereafter, the scoured fabrics are bleached with hydrogen peroxide together with detergents and wetting agents to remove the protoplasmic residues of protein and flavone pigments of cotton. The chemicals used in the pre-processing stage are characterized by high levels of chemical oxygen demand (COD), biological oxygen demand (BOD), low pH and toxicity, which are present in the wastewater effluent produced by these processes, which is often discharged into the environment.

The processing stage involves the dying or printing of fabrics using dyes together with other chemicals to improve the adsorption process between the colors and fibres. Synthetic dyes are commonly used for this purpose due to their low cost, brilliant shade and variety of colors. However, unfixed residual synthetic dyes present in the wastewater effluent produced by the processing of cotton fabrics have been shown to be both toxic and carcinogenic (Zaharia et al., 2009, Environmental Engineering and Management Journal, 8: 1359-1369).

Consumer demand for more sustainably produced textiles has resulted in textile industries implementing cleaner production technologies, effluent treatment strategies and the reemergence of the use of natural dyes. However, effluent treatment strategies have proven to be expensive and ineffective to reduce chemical pollutants (see, e.g., Ciardelli et al., 2001, Resources, Conservation and Recycling, 31(2): 189-197), and the requirement for mordants (e.g., heavy metals, such as aluminum, iron, tin, copper and chrome, or acidic or basic chemical agents) to adhere natural dyes to cotton has limited the use of such natural dyes in the processing of cotton fabrics (see, e.g., Prabhu and Bhute, 2012, Journal of Natural Product and Plant Resources, 2(6): 649-664).

Naturally colored cotton has been known for more than 5000 years and occurs in all four species of cultivated cotton (i.e., Gossypium hirsutum, Gossypium barbadense, Gossypium herbaceum and Gossypium arboretum). However, these colored varieties generally have low-yield, poor fibre quality and variable and unstable colors. Although conventional breeding has improved the properties of some colored cotton, quality and yield remain low compared to white cotton and color range is limited. Genetically modifying cotton plants to alter the color of cotton fibre provides an alternative to the use of synthetic dyes, without the fibre quality limitations of naturally colored cotton. Previous approaches to genetically modify cotton to produce colored fibre include the introduction of indigoidine biosynthesis genes (i.e., igiA, igiD and igiB) to produce a blue pigment. However, constitutive expression of the indigoidine biosynthesis genes throughout the plant was toxic (Van de Loo, 2000). Moreover, other approaches to express transgenic pigment synthesis genes have attempted to produce cotton fibre with vivid colors, e.g., red, but have only produced light brown fiber (see, e.g., U.S. Pat. No. 7,732,678).

There remains, therefore, a need for improved methods and means for the sustainable production of colored fibre and associated products derived therefrom.

### SUMMARY OF THE DISCLOSURE

In an aspect of the present disclosure there is provided a nucleic acid construct comprising a nucleotide sequence encoding one or more pigment producing genes, wherein expression of the nucleotide sequence is under the control of a cotton fibre-specific promoter, and wherein the cotton fibre-specific promoter is capable of driving expression of the nucleotide sequence during two or more fibre development stages.

In another aspect, the present disclosure provides a nucleic acid construct comprising a nucleotide sequence comprising one or more pigment producing genes, wherein the one or more pigment producing genes are exogenous pigment producing genes that are non-native to cotton, wherein expression of the nucleotide sequence is under the control of a cotton fibre-specific promoter, wherein the cotton fibre-specific promoter is capable of driving expression of the nucleotide sequence during two or more fibre development stages, and wherein the cotton fibre-specific promoter is capable of driving expression of the nucleotide sequence at a level that is sufficient to produce pigment that is present in cotton fibre at ≥40 DPA.

In another aspect, the present disclosure provides a vector comprising the nucleic acid construct described herein.

In another aspect, the present disclosure provides a cell comprising the nucleic acid construct or vector described herein.

In another aspect, the present disclosure provides a polypeptide encoded by the nucleic acid construct described herein.

In another aspect, the present disclosure provides a method of producing a colored cotton fibre, the method comprising: (i) transforming a cotton plant cell with the nucleic acid construct or vector described herein; (ii) regenerating a cotton plant from the transformed cell; and (iii) harvesting the fibre from the regenerated cotton plant.

In another aspect, the present disclosure provides a method of introducing one or more exogenous pigment producing genes into a cotton plant, wherein the one or more pigment producing genes are heterologous to the cotton plant, the method comprising:
(i) crossing a first parent plant with a second parent plant, wherein said second plant is transgenic for the nucleic acid construct described herein; and
(ii) backcrossing a progeny plant of the cross of step (i) with a plant of the same genotype as the first parent plant to produce a cotton plant with a majority of the genotype of the first parent plant but characterized by the expression of the one or more pigment producing genes in the fibre cells of the cotton plant.

In another aspect, the present disclosure provides a plant modified to produce colored fibre, wherein the plant comprises the nucleic acid construct described herein, and wherein the one or more pigment producing genes are expressed in fibre cells of the plant.

In another aspect, the present disclosure provides a cotton plant modified to produce colored fibre, wherein the cotton plant comprises the nucleic acid construct described herein, and wherein the one or more pigment producing genes are expressed in fibre cells of the cotton plant.

In another aspect, the present disclosure provides a method of producing cotton seed, wherein the method comprises crossing the cotton plant described herein with itself, or a second, distinct cotton plant to produce cotton seed.

In another aspect, the present disclosure provides cotton seed produced by the methods described herein.

In another aspect, the present disclosure provides a composition comprising the cotton seed described herein, wherein the seed is comprised in plant seed growth media, and wherein said plant seed growth media comprises soil or synthetic cultivation media.

In another aspect, the present disclosure provides a method of producing a plant commodity product, wherein the method comprises collecting the plant commodity product from the cotton plant described herein.

In another aspect, the present disclosure provides a method of producing the cotton lint described herein, the method comprising: (i) growing the cotton plant described herein; and (ii) harvesting the cotton lint.

In another aspect, the present disclosure provides cotton lint obtained from the cotton plant described herein, or produced by the methods described herein.

In another aspect, the present disclosure provides a method of producing yarn, the method comprising: (i) obtaining the cotton lint described herein; and (ii) spinning the cotton lint to produce the yarn.

In another aspect, the present disclosure provides a yarn comprising the cotton lint described herein, or produced by the methods described herein.

In another aspect, the present disclosure provides a textile comprising the yarn described herein.

In another aspect, the present disclosure provides a method to produce a textile, the method comprising: (i) obtaining the yarn described herein; and (ii) weaving or knitting the yarn into a fabric to produce the textile.

### BRIEF DESCRIPTION OF THE DRAWINGS

Embodiments of the disclosure are described herein, by way of non-limiting example only, with reference to the accompanying drawings.

FIG. 1 shows the recombinant expression of betalain genes in Nicotiana benthamiana and Gossypium hirsutum. (A) A photographic representation of transient expression of betalain proteins in Nicotiana benthamiana by Agrobacterium-mediated infiltration of vectors comprising purple (DODA1, CYP76AD1 and cDOPA5GT) or yellow/orange (DODA1 and CYP76AD6) betalain gene constructs driven by the 2×35S constitutive promoter as shown in bottom left corner (2001), bottom right corner (2003) and top right corner (P19, negative control) and a vector comprising the purple betalain gene construct driven by the ltp3/8K12 fibre-specific promoter as shown in the top left corner (2009). (B) A photographic representation of cotton callus (left panel) and young plants/plantlets (right panel) transformed stably transformed with the P19 negative control construct. (C) A photographic representation of cotton callus (left panel) and young plants/plantlets (right panel) transformed stably transformed with a construct comprising the coding sequence of DODA1, CYP76AD1 and cDOPA5GT driven by the 2×35S constitutive promoter to generate purple pigment (2001). (D) A photographic representation of cotton callus (left panel) and young plants/plantlets (right panel) transformed stably transformed with a construct comprising the coding sequence of DODA1 and CYP76AD6 driven by the 2×35S constitutive promoter to generate yellow/orange pigment (2003).

FIG. 2 shows the difference in the expression of betalain genes in the whole plant and fibre tissue of cotton plants. (A) A photographic representation of a control cotton plant. (B) A photographic representation of a T₀ cotton plant stably transformed with a construct comprising the coding sequence of DODA1, CYP76AD1 and cDOPA5GT driven by the 2×35S constitutive promoter to generate purple pigment. (C) A photographic representation of a T₀ cotton plant stably transformed with a construct comprising the coding sequence of DODA1, CYP76AD1 and cDOPA5GT driven by the ltp3/8K12 fibre-specific promoter to generate purple pigment. (D)-(F) A series of photographic representations of cotton boll derived from the (D) control cotton plant; (E) T₀ cotton plant stably transformed with a construct comprising the coding sequence of DODA1, CYP76AD1 and cDOPA5GT driven by the 2×35S constitutive promoter; and (F) T₀ cotton plant stably transformed with a construct comprising the coding sequence of DODA1, CYP76AD1 and cDOPA5GT driven by the ltp3/8K12 fibre-specific promoter. (G) A photographic representation of the pink fibres of a cotton boll derived from a cotton plant stably transformed with a construct comprising the coding sequence of DODA1, CYP76AD1 and cDOPA5GT driven by the ltp3/8K12 fibre-specific promoter at 39 days post-anthesis (DPA) as compared to a control cotton boll with white fibres at around 40 DPA. (H) A photographic representation of two freeze-dried cotton bolls from wild type control cotton plants (left) and a cotton plant stably transformed with a construct comprising the coding sequence of DODA1, CYP76AD1 and cDOPA5GT driven by the ltp3/8K12 fibre-specific promoter (right) at 47 DPA. (I) A photographic representation of fresh cotton bolls of wild type control plants (Coker 315) and cotton plants stably transformed with a construct comprising the coding sequence of DODA1, CYP76AD1 and cDOPA5GT driven by the ltp3/8K12 fibre-specific promoter fibre-specific promoter at different developmental stages. All bolls were harvested within two days of the stated DPA (22 and 27 DPA bolls are at the precise stage). (J) A photographic representation of freeze-dried cotton bolls of wild type control plants (Coker 315) and cotton plants stably transformed with a construct comprising the coding sequence of DODA1, CYP76AD1 and cDOPA5GT driven by the ltp3/8K12 fibre-specific promoter fibre-specific promoter at different developmental stages. All bolls were harvested within two days of the stated DPA (22 and 27 DPA bolls are at the precise stage). (K) A photographic representation of the red cells of a leaf from the transgenic cotton plants stably transformed with a construct comprising the coding sequence of DODA1, CYP76AD1 and cDOPA5GT driven by the 2×35S constitutive promoter to generate purple pigment. Image obtained under magnification. (L) A series of photographic representations of the fluorescence signal of betalain pigment inside cotton leaf cells of plants stably transformed with a construct comprising the coding sequence of DODA1, CYP76AD1 and cDOPA5GT driven by the 2×35S constitutive promoter to generate purple pigment. Images obtained by confocal imaging. (M) A series of photographic representations of the fluorescence signal of betalain pigment inside cotton fibre cells stably transformed with a construct comprising the coding sequence of DODA1, CYP76AD1 and cDOPA5GT driven by the ltp3/8K12 fibre-specific promoter. Images obtained by confocal imaging.

FIG. 3 shows the recombinant expression of amilGFP in Nicotiana benthamiana and cotton. (A) A photographic representation of transient expression of amilGFP proteins in Nicotiana benthamiana by Agrobacterium-mediated infiltration of constructs driven by the 2×35S constitutive promoter (bottom) as compared to control (top) at 7 days post-infiltration (left panel); and a series of photographic representations of the fluorescent signal of amilGFP in the leaf of Nicotiana benthamiana transiently expressing amilGFP proteins following infiltration with a construct comprising the coding sequence of amilGFP driven by the 2×35S constitutive promoter at 3 days post-infiltration (right panel). (B) A series of photographic representations of the fluorescence signal of amilGFP protein in cotton leaf cells of plants stably transformed with a construct comprising the coding sequence of amilGFP driven by the 2×35S constitutive promoter, with or without a signal peptide obtained by confocal imaging. (C) A series of photographic representations of cytosolic expression of amilGFP in young cotton fibres of plants stably transformed with a construct comprising the coding sequence of amilGFP driven by the 2×35S constitutive promoter. (D) A series of photographic representations of cell wall expression of amilGFP in young cotton fibres of plants stably transformed with a construct comprising the coding sequence amilGFP driven by the 2×35S constitutive promoter and a signal peptide obtained by confocal imaging.

FIG. 4 shows the fineness and maturity ratio of cotton fibres measured with CottonScope. (A) A graphical representation of the fineness (y-axis; mTex) of mature and freeze-dried cotton fibre derived from wild type cotton plants or cotton plants stably transformed with a construct comprising the coding sequence of DODA1, CYP76AD1 and cDOPA5GT driven by the ltp3/8K12 fibre-specific promoter (x-axis). (B) A graphical representation of the maturity ratio (y-axis) of mature and freeze-dried cotton fibre derived from wild type cotton plants or cotton plants stably transformed with a construct comprising the coding sequence of DODA1, CYP76AD1 and cDOPA5GT driven by the ltp3/8K12 fibre-specific promoter. The mean is indicated by filled black diamonds. The letters (i.e., a, b, c, d, etc.) indicate significant differences between genotypes in A and between data sets in B, based on one-way ANOVA analysis with Fisher's LSD post hoc test.

FIG. 5 is a schematic representation of cotton fibre development adapted from Basra and Malik (1984, International Review of Cytology, 89: 65-113). As shown, cotton fibre development is classified into four overlapping stages: fibre initiation (from 3 days before anthesis to 5 days post-anthesis (DPA)), elongation (5 to 25 DPA), secondary cell wall deposition (from 15 to 45 DPA), and maturation (from 40 to 45 DPA, and thereafter).

FIG. 6 is a schematic representation of the ltp3/8K12 BAASS-amilGFP pMDC32 vector.

FIG. 7 is a schematic representation of the ltp3/8K12 BAASS-eforRED pMDC32 vector.

FIG. 8 is a schematic representation of the ltp3/8K12 DODA1-CYP76AD1-cDOPA5GT pMM2009 vector.

FIG. 9 shows that drying of immature cotton bolls is sufficient to maintain exogenous pigment in the fibre of transgenic cotton plants. A photographic representation of immature cotton bolls (50 or 51 DPA) with seeds from three independent transgenic lines (T1, T2, T3) that have been freeze dried (top row) or dried at room temperature (bottom row).

FIG. 10 shows that drying of fresh ginned wet cotton fibres is sufficient to maintain exogenous pigment in the fibre of transgenic cotton plants. (A) A photographic representation of fresh ginned cotton fibres from transgenic line T2 that have been freeze dried or dried in an oven with fan as compared to control wet fibres kept at room temperature. (B) A photographic representation of fresh ginned cotton fibres from transgenic line T2 that have been hot plate dried as compared to control wet fibres kept at room temperature. All groups of fibres were from the same immature cotton boll (˜45 DPA) and hand ginned to remove the seeds.

### BRIEF DESCRIPTION OF THE SEQUENCES

Nucleic acid sequences are referred to by a sequence identifier number (SEQ ID NO), with reference to the accompanying sequence listing.

SEQ ID NO: 1 shows the nucleotide sequence of cDOPA5GT (Mirabilis jalapa) codon optimized for expression in Arabidopsis.

SEQ ID NO: 2 shows the amino acid sequence of cDOPA5GT (Mirabilis jalapa).

SEQ ID NO:3 shows the nucleotide sequence of DODA1 (Beta vulgaris) codon optimized for expression in Arabidopsis.

SEQ ID NO: 4 shows the amino acid sequence of DODA1 (Beta vulgaris).

SEQ ID NO: 5 shows the nucleotide sequence of CYP76AD1 (Beta vulgaris) codon optimized for expression in Arabidopsis.

SEQ ID NO: 6 shows the amino acid sequence of CYP76AD1 (Beta vulgaris).

SEQ ID NO: 7 shows the nucleotide sequence of CYP76AD6 (Beta vulgaris) codon optimized for expression in Arabidopsis.

SEQ ID NO: 8 shows the amino acid sequence of CYP76AD6 (Beta vulgaris).

SEQ ID NO: 9 shows the nucleotide sequence of amilGFP.

SEQ ID NO: 10 shows the amino acid sequence of amilGFP.

SEQ ID NO: 11 shows the nucleotide sequence of CBDclos codon optimized for expression in E. coli.

SEQ ID NO: 12 shows the amino acid sequence of CBDclos.

SEQ ID NO: 13 shows the nucleotide sequence of the 2×35S promoter in the pAGM4723 construct.

SEQ ID NO: 14 shows the nucleotide sequence of the 2×35S promoter in the pMDC32 construct.

SEQ ID NO: 15 shows the nucleotide sequence of the ltp3/8K12 promoter.

SEQ ID NO: 16 shows the nucleotide sequence of amilCP codon optimized for expression in Arabidopsis.

SEQ ID NO: 17 shows the nucleotide sequence of amilCP codon optimized for expression in E. coli.

SEQ ID NO: 18 shows the amino acid sequence of amilCP (Acropora millepora).

SEQ ID NO: 19 shows the nucleotide sequence of eforRED codon optimized for expression in Arabidopsis.

SEQ ID NO: 20 shows the nucleotide sequence of eforRED codon optimized for expression in E. coli.

SEQ ID NO: 21 shows the amino acid sequence of eforRED.

SEQ ID NO: 22 shows the nucleotide sequence encoding the BAASS signal peptide.

SEQ ID NO: 23 shows the nucleotide sequence encoding the BAASS signal peptide codon optimized for expression in Arabidopsis.

SEQ ID NO: 24 shows the amino acid sequence of the BAASS signal peptide.

SEQ ID NO: 25 shows the nucleotide sequence encoding the GhFLA7 signal peptide.

SEQ ID NO: 26 shows the amino acid sequence of the GhFLA7 signal peptide.

SEQ ID NO: 27 shows the nucleotide sequence encoding the cotton FLA12 signal peptide.

SEQ ID NO: 28 shows the amino acid sequence of the cotton FLA12 signal peptide.

SEQ ID NO: 29 shows the nucleotide sequence of the cotton FLA7 promoter.

SEQ ID NO: 30 shows the nucleotide sequence of the cotton FLA12 promoter.

SEQ ID NO: 31 shows the nucleotide sequence of the ltp3/8K12-DODA1-ltp3/8K12-CYP76AD1-ltp3/8K12-cDOPA5GT construct.

SEQ ID NO: 32 shows the nucleotide sequence of the CYP76AD1-DODA1-cDOPA5GT construct.

SEQ ID NO: 33 shows the nucleotide sequence of the pMM2001 vector.

SEQ ID NO: 34 shows the nucleotide sequence of the pMM2003 vector.

SEQ ID NO: 35 shows the nucleotide sequence of the pMM2009 vector.

SEQ ID NO: 36 shows the nucleotide sequence of the ltp3/8K12 BAASS-amilGFP pMDC32 vector.

SEQ ID NO: 37 shows the nucleotide sequence of the ltp3/8K12 BAASS-eforRED pMDC32 vector.

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “an allele” includes a single allele, as well as two or more alleles; reference to “a treatment” includes a single treatment, as well as two or more treatments; and so forth.

In the context of this specification, the term “about” in relation to a numerical value or range is intended to cover numbers falling within ±10% of the specified numerical value or range.

Each embodiment in this specification is to be applied mutatis mutandis to every other embodiment, unless expressly stated otherwise.

Genes and other genetic material (e.g., mRNA, constructs, etc.) are represented in italics and their proteinaceous expression products are represented in non-italicized form. Thus, for example, amilGFP is an expression product of amilGFP.

Nucleotide and amino acid sequences are referred to by a sequence identifier number (i.e., SEQ ID NO:). The SEQ ID NOs: correspond numerically to the sequence identifiers <400>1 (SEQ ID NO: 1), <400>2 (SEQ ID NO: 2), etc. A sequence listing is provided after the claims. A list describing the SEQ ID NOs in the sequence listing is provided above under the section “Brief Description of the Sequences”.

All sequence identifiers (e.g., GenBank ID, EMBL-Bank ID, DNA Data Bank of Japan (DDBJ) ID, etc.) provided herein were current at the filing date.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

The present invention is based in part on the surprising observations made in the experiments described herein that exogenous pigments, such as carotenoids, anthocyanins, chromoproteins or betalains, can be produced in the fibre cells of plants by the expression of a nucleic acid construct comprising a nucleotide sequence encoding one or more pigment producing genes, wherein expression of the nucleotide sequence is under the control of a fibre-specific promoter that is capable of driving expression during two or more fibre development stages. In certain embodiments described herein, the selective expression of the one or more pigment producing genes in fibre cells avoids any negative impacts, e.g., on plant growth and development, which may result from constitutive expression of the one or more pigment producing genes throughout the entire plant. By generating colored fibre in planta, the inventors have beneficially developed a more sustainable and less toxic approach for producing colored fibre, as compared to current methods for scouring and dying fibre (or lint or yarn comprising such fibres).

### Nucleic Acid Molecules and Constructs

Accordingly, in an aspect, the present disclosure provides a nucleic acid construct comprising a nucleotide sequence encoding one or more pigment producing genes, wherein expression of the nucleotide sequence is under the control of a fibre-specific promoter, and wherein the fibre-specific promoter is capable of driving expression of the nucleotide sequence during two or more or all of fibre development stages.

As used herein, a “nucleic acid construct” refers to any nucleic acid molecule that comprises nucleotide sequences that are not a native gene in its native location. Typically, a nucleic acid construct comprises regulatory sequences (e.g., a fibre-specific promoter) and transcribed or protein coding sequences (e.g., one or more pigment producing genes) that are not found together in nature. The nucleic acid construct may comprise regulatory nucleotide sequences and coding nucleotide sequences that are derived from different sources.

The term “endogenous” is used herein to refer to a substance that is normally present or produced in an unmodified plant at the same developmental stage as the plant under investigation. An “endogenous gene” thus refers to a native gene in its natural location in the genome of an organism. As used herein, “recombinant nucleic acid molecule” may be used interchangeably with “nucleic acid construct” to refer to a nucleic acid molecule that has been constructed or modified by recombinant DNA technology. The term “exogenous” is used herein to refer to a substance that is not normally present or produced in an unmodified plant at the same developmental stage as the plant under investigation. The terms “exogenous nucleic acid molecule” or “exogenous nucleotide sequence” and the like refer to any nucleic acid which is introduced into the genome of a cell by experimental manipulations. Foreign or exogenous genes may be genes that are inserted into a non-native organism, native genes introduced into a new location within the native host, or chimeric genes. A “transgene” is a gene that has been introduced into the genome by a transformation procedure. The term “genetically modified” included introducing genes into cells by transformation or transduction, mutating genes in cells and altering or modulating the regulation of a gene in a cell or organisms to which these acts have been done or their progeny.

As used herein, a “polynucleotide”, “nucleic acid”, “nucleic acid molecule” or “nucleotide sequence” means a polymer of nucleotides, which may be DNA or RNA or a combination thereof, and includes mRNA, cRNA, cDNA, tRNA, siRNA, shRNA and hpRNA. It may be DNA or RNA of cellular, genomic or synthetic origin, for example, made with an automated synthesizer, and may be combined with carbohydrate, lipids, protein or other materials, labelled with fluorescent or other groups, or comprise one or more modified nucleotides not found in nature, well known to those skilled in the art. The polymer may be single-stranded, essentially double-stranded or partly double-stranded. Examples of a partly-double-stranded RNA molecule include a hairpin RNA (hpRNA), short hairpin RNA (shRNA) or self-complementary RNA, which comprise a double-stranded stem formed by base pairing between a nucleotide sequence and its complement and a loop sequence which covalently joins the nucleotide sequence and its complement. “Base pairing” as used herein refers to standard base pairing between nucleotides, include G:U base pairs. “Complementary” means two polynucleotides are capable of base pairing (hybridizing) along part of their lengths, or along the full length of one or both. A “hybridized polynucleotide” means the polynucleotide is actually base paired to its complement.

By “isolated” it is meant material that is substantially free or essentially free from components that normally accompany it in its native state. As used herein, an “isolated nucleic acid molecule” or “isolated polynucleotide” means a nucleic acid molecule which is at least partially separated from, preferably substantially or essentially free of, the polynucleotide sequences of the same type with which it is associated or linked in its native state. For example, an “isolated nucleic acid molecule” includes a nucleic acid molecule which has been purified or separated from the sequences which flank it in a naturally occurring state, e.g., a DNA fragment which has been removed from the sequences which are normally adjacent to the fragment. Preferably, the isolated nucleic acid molecule is at least 90% free from other components, such as proteins, carbohydrates, lipids, etc. The term “nucleic acid construct” as used herein refers to a nucleic acid molecule formed in vitro by the manipulation of nucleic acid into a form not normally found in nature.

In another aspect, the present disclosure provides an isolated nucleic acid construct comprising a nucleotide sequence encoding one or more pigment producing genes, wherein expression of the nucleotide sequence is under the control of a fibre-specific promoter, and wherein the fibre-specific promoter is capable of driving expression of the nucleotide sequence during two or more fibre development stages.

The term “gene” as used herein is to be taken in the broadest context to include the DNA sequences comprising the protein coding region of a structural gene and sequences located adjacent to the coding region on both the 5′ and 3′ ends for a distance of at least about 2 kilobases (kb) on either end. The sequences which are located 5′ of the coding region and which are present on the mRNA are referred to as “5′ non-translated sequences”. The sequences which are located 3′ or downstream of the coding region and which are present on the mRNA are referred to as 3′ non-translated sequences. The term “gene” encompasses by cDNA are genomic forms of a gene. A genomic form or clone of a gene contains the coding region which may be interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide. The term “gene” includes a synthetic or fusion molecules encoding all or part of the proteins of the invention described herein and a complementary nucleotide sequence to any one of the above.

A “pigment producing gene” refers to a nucleotide sequence encoding a pigment or enzymes capable of producing a pigment. As the pigment producing genes contemplated herein are derived from natural sources, e.g., coral, plants of the order Caryophyllales and mushrooms of the genera Amanita, Hygrocybe and Hygrophorus, pigments encoded by such genes, or produced by the enzymes encoded by such genes may thus be referred to as “natural pigments”.

In an embodiment, the one or more pigment producing genes encodes a pigment or an enzyme required to produce a pigment.

In an embodiment, the one or more pigment producing genes are exogenous pigment producing genes. In an embodiment, the one or more exogenous pigment producing genes are non-native to cotton.

By “pigment” it is meant any light absorbing molecule. The color of a pigment is determined by the wavelengths of light that are reflected (i.e., not absorbed) by the molecule. The term “pigment” is used herein in the broadest possible manner to encompass molecules that absorb/reflect visible or non-visible wavelengths of light.

In an embodiment, the pigment is an exogenous pigment. Exogenous pigments may be pigments that are non-native to the organism (i.e., not naturally produced by the organism), or pigments that are not typically expressed in fibre cells, or at a level sufficient to generate colored fibre.

In an embodiment, the exogenous pigments are non-native to cotton.

In an embodiment, the pigment is melanin.

In an embodiment, the pigment is selected from the group consisting of a chromoprotein, a betalain, an anthocyanin, a carotenoid and an indigoidine.

The term “chromoprotein” as used herein refers to a family of eukaryotic proteins that are produced by, e.g., corals, fungi and sea anemone. For example, in corals, these small proteins are homologous with green fluorescent protein (GFP) produced by jellyfish and are encoded by single genes, which assemble their chromophore without the need for co-factors or other substrates. Suitable chromoproteins would be known to persons skilled in the art, illustrative examples of which include meffRED (Montipora efflorescens), eforRED (Echinopora forskaliana), asPink (Anemonia sulcata), spisPink (Stylophora pistillata), amilGFP (Acropora millepora), amajLime (Anemonia majano), cjBlue (Cnidopus japonicas), meffBlue (Montipora efflorescens), aeBlue (Actinia equina), amilCP (Acropora millepora) and gfasPurple (Galaxea fascicularis).

In an embodiment, the chromoprotein is selected from the group consisting of amilGFP (Acropora millepora), amilCP (Acropora millepora), eforRED (Echinopora forskaliana), and combinations of the foregoing.

The term “betalain” as used herein refers to vacuolar pigments that are produced by plants of the order Caryophyllales and mushrooms of the genera Amanita, Hygrocybe and Hygrophorus.

In an embodiment, the betalain is a betacyanin.

“Betacyanins” contain a cyclo-3,4-dihydroxyphenylalanine (cylo-DOPA) residue, and are red-violet in color. The most common betacyanin is betanin (i.e., betanidin 5-O-β-glucoside, or “beetroot red”), however, other betacyanins would be known to persons skilled in the art, illustrative examples of which include isobetanin, neobetanin, 2-decarboxy-neobetanin, 2,17-bidecarboxy-betanin, 17-decarboxy-neobetanin, 6′-O-feruloyl-betanin and 6′-O-feruloyl-isobetanin isobetanin.

In an embodiment, the betacyanin is betanin.

In an embodiment, the betalain is a betaxanthin.

“Betaxanthins” contain different amino acid or amine residues, and are yellow-orange in color. Suitable betaxanthins would be known to persons skilled in the art, illustrative examples of which include vulgaxanthin I, miraxanthin V, indicaxanthin and dopaxanthin.

In an embodiment, the betaxanthin is vulgaxanthin I.

The term “indigoidine” as used herein refers to a natural blue pigment that is produced by several bacteria. The biosynthetic pathway of indigodine has been identified in multiple bacterial species, including Erwinia chrysantemi, Photohabdus luminescens, Phaeobacter spp., Streptomyces chromofuscus and Vogesella indigofera. In one example, indigodine synthesis in Erwinia chrysantemi is dependent on a regulator region (pecS) and three open reading frames (ORFs) designated indA, indB and indC. In Vogesella indigofera, the indigoidine locus is composed of five genes, igiA, igiB, igiC, igiD and igiE.

In an embodiment, the one or more pigment producing genes is selected from the group consisting of cDOPA5GT, DODA1, CYP76AD1, CYP76AD6, amilGFP, amilCP, eforRED, igiA, igiD, igiB and combinations of the foregoing.

In an embodiment, the one or more pigment producing genes is selected form the group consisting of cDOPA5GT, DODA1, CYP76AD1, CYP76AD6 and combinations of the foregoing.

cDOPA5GT encodes a cyclo-DOPA 5-O-glucosyltransferase, which glycosylates the 5-O-ring position of cyclo-DOPA to produce cyclo-DOPA-glucoside, which then spontaneously condenses with betalamic acid to form betalain. An example of a cDOPA5GT gene is that of Mirabilis jalapa (GenBank ID: AB182643.1). An example of a codon optimized variant of cDOPA5GT is the sequence shown herein as SEQ ID NO: 1.

DODA1 encodes a L-DOPA 4,5-dioxygenase, which converts L-DOPA into betalamic acid, which can spontaneously condense with amino acids to form betaxanthins. An example of a DODA1 gene is that of Beta vulgaris (GenBank ID: HQ656027.1). An example of a codon optimized variant of CYP76AD6 is the sequence shown herein as SEQ ID NO: 3.

CYP76AD1 encodes a Cytochrome P450-like protein, which oxidizes L-DOPA, to form cyclo-DOPA. An example of a CYP76AD1 gene is that of Beta vulgaris (GenBank ID: HQ656023.1). An example of a codon optimized variant of CYP76AD6 is the sequence shown herein as SEQ ID NO: 5.

CYP76AD6 encodes a Cytochrome P450-like protein, which catalyzes tyrosine hydroxylation, to form L-DOPA. An example of a CYP76AD6 gene is that of Beta vulgaris (GenBank ID: KT962274.1). An example of a codon optimized variant of CYP76AD6 is the sequence shown herein as SEQ ID NO: 7.

In an embodiment, the one or more pigment producing genes encode a red-violet pigment or enzymes required to produce a red-violet pigment (e.g., DODA1, CYP76AD1 and cDOPA5GT). In accordance with this embodiment, the one or more pigment producing genes comprise DODA1, CYP76AD1 and cDOPA5GT.

In an embodiment, the one or more pigment producing genes encode a yellow-orange pigment or the enzymes required to produce a yellow-orange pigment. In accordance with this embodiment, the one or more pigment producing genes comprise DODA1 and CYP76AD6.

In an embodiment, the one or more pigment producing genes encode an orange-pink pigment or the enzymes required to produce an orange-pink pigment. In accordance with this embodiment, the one or more pigment producing genes comprise cDOPA5GT, DODA1, CYP76AD1 and CYP76AD6.

In an embodiment, the one or more pigment producing genes is selected from the group consisting of amilGFP, amilCP, eforRED and combinations of the foregoing.

amilGFP encodes a green fluorescent protein comprising the FQYG chromophore derived from Acropora millepora (UniProt ID: Q66PV8). An example of a codon optimized variant of amilGFP is the sequence shown herein as SEQ ID NO: 9.

amilCP encodes a violet fluorescent protein comprising the CQYG chromophore derived from Acropora millepora (UniProt ID: Q66PV0).

eforRED encodes a red fluorescent protein comprising the HMYG chromophore derived from Echinopora forskalilana (GenPept ID: ACD13196).

In an embodiment, the pigment producing gene is amilGFP.

Where the nucleic acid construct comprises a nucleotide sequences encoding more than one pigment producing gene, persons skilled in the art will appreciate that the pigment producing genes may be arranged in tandem in the nucleic acid construct, e.g., in the same reading frame, such that the expression of the pigment producing genes may be placed under the control of a single fibre-specific promoter (see, e.g., SEQ ID NO: 32), or the pigment producing genes may be arranged with multiple fibre-specific promoters, such that the expression of each pigment producing gene is under the control of a fibre-specific promoter (see, e.g., SEQ ID NO: 31).

The term “fibre-specific promoter” refers to an array of nucleic acid control sequences that direct transcription of the nucleotide molecule that is preferentially expressed in fibre cells relative to other tissues, cells or organs, preferably most if not all other tissues or organs in a plant.

As used herein, the term “fibre cell” refers to plant cells with thick secondary cell walls, including cells present in the seed trichome and plant xylem, phloem or stem. In some embodiments, the fibre cells are cells of the cotton plant seed fibre, cotton xylem and phlomen fibres, bast fibres, hemp seed fibres and bamboo stem fibres.

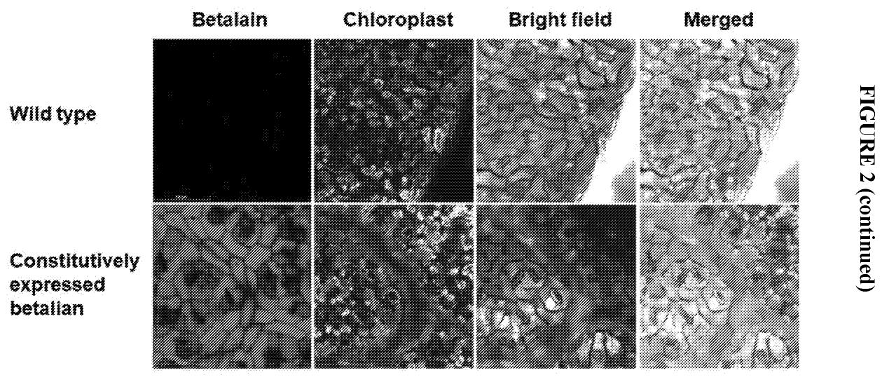

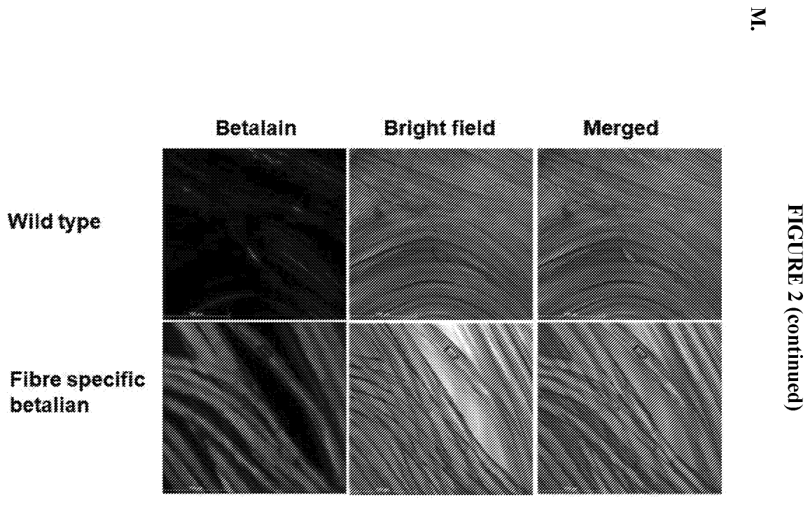

The fibre-specific promoters contemplated herein are capable of driving expression of the nucleotide sequence during two or more fibre development stages. Fibre development stages define the process by which fibre cells are produce and maturate to form mature fibres. For example, cotton fibre development undergoes several distinctive, but overlapping steps including fibre initiation, elongation, secondary wall deposition and maturation, as shown in FIG. 5.

The selection of fibre-specific promoters that are capable of driving expression during two or more fibre development stages beneficially enables the production and accumulation of higher concentrations of pigments, results in the generation of vivid colored fibre until very late stages of fibre development, as compared to fibre-specific promoters that only drive expression in one fibre development stage, e.g., the fibre elongation-specific promoter, PGbEXPA2 (Li et al., 2015, Plant Cell Reports, 34: 1539-1549), which only drives expression in 5-10 DPA fibres.

In an embodiment, the fibre-specific promoter is a cotton fibre-specific promoter. Suitable cotton fibre-specific promoters would be known to persons skilled in the art, illustrative examples of which include ltp3/8K12, GhSCFP, and the promoter of the cotton fibre-specific genes CesA, CSL FLA, FSltp4, Expansin, E6, Rac13, CelA1, LTP, and Fb late as described by, e.g., MacMillan et al., (2017, BMC Genomics, 18: 539), Chen and Burke (2015, PLoS One, 10(6): e0129870), Delaney et al. (2007, Plant Cell Physiology, 48(10): 1426-1437) and Yaqoob et al. (2020, PLoS One, 15(3): e0230519).

In an embodiment, the cotton fibre-specific promoter is ltp3/8K12, GhSCFP, or the promoter of a cotton fibre-specific gene selected from the group consisting of FSltp4, Expansin, E6, Rac13, FLA7, FLA12, FLA9, FLA11, FLA17, CelA1, LTP, and Fb late.

In an embodiment, the cotton-fibre-specific promoter is ltp3/8K12 or the promoter of cotton-fibre-specific gene selected from the group consisting of FLA7, FLA12, FLA9, FLA11 and FLA17.

In an embodiment, the cotton fibre-specific promoter is ltp3/8K12 (SEQ ID NO: 15; as described in US Patent Application No. 2017/0247712, the content of which is incorporated herein).

In another embodiment, the cotton fibre-specific promoter is the promoter of cotton fibre-specific gene selected from the group consisting of FLA7, FLA12, FA9, FLA11 and FLA17.

In an embodiment, the cotton fibre-specific promoter is capable of driving expression of the nucleotide sequence during two of the fibre development stages selected from fibre initiation (i.e., 0-5 DPA), elongation (i.e., 5-25 DPA), secondary wall deposition (i.e., 15-45 DPA), maturation (i.e., ≥40 DPA), and any combination of the foregoing.

In an embodiment, the cotton fibre-specific promoter is capable of driving expression of the nucleotide sequence during two of the fibre development stages selected from (a) fibre initiation, being about 0-about 5 DPA; (b) elongation, being about 5-about 25 DPA; (c) secondary wall deposition, being about 15-about 45 DPA; and (d) maturation, being at least about 40 DPA, and (e) any combination of (a) to (d).

In an embodiment, the cotton fibre-specific promoter is capable of driving expression of the nucleotide sequence during fibre initiation, being about 0-about 5 DPA (e.g., 0, 1, 2, 3, 4 or 5 DPA).

In an embodiment, the cotton fibre-specific promoter is capable of driving expression of the nucleotide sequence during fibre elongation, being about 5-about 25 DPA (e.g., 5, 6, 7, 8, 9, 10, 11, 12, 13, 14, 15, 16, 17, 18, 19, 20, 21, 22, 23, 24 or 25 DPA).

In an embodiment, the cotton fibre-specific promoter is capable of driving expression of the nucleotide sequence during secondary wall deposition, being about 15-about 45 DPA (e.g., 15, 16, 17, 18, 19, 20, 21, 22, 23, 24, 25, 26, 27, 28, 29, 30, 31, 32, 33, 34, 35, 36, 37, 38, 39, 40, 41, 42, 43, 44 or 45 DPA).

In an embodiment, the at least two fibre development stages at elongation and secondary wall deposition.

In an embodiment, the cotton fibre-specific promoter is capable of driving expression of the nucleotide sequence from about 14 DPA to about 45 DPA.

In an embodiment, the cotton fibre-specific promoter is capable of driving expression of the nucleotide sequence at a level that is sufficient to produce pigment that is present in cotton fibre at ≥40 DPA.

The nucleic acid construct may comprise additional regulatory elements of sequences. Suitable regulatory sequences would be known to persons skilled in the art, illustrative examples of which include leader or signal sequences, ribosomal binding sites, transcriptional start and termination sequences, and enhancer or activator sequences.

It is also contemplated herein that the nucleic acid construct comprise nucleotide sequences encoding elements and amino acid sequences associated with protein localization and interactions. Accordingly, in an embodiment, the nucleic acid construct further comprises a nucleotide sequence encoding one or both of (i) a signal peptide; and (ii) an anchoring domain.

By “signal peptide” it is meant an amino acid sequence of a plant protein, which is typically located at the N-terminus of the protein, and directs the protein from the cytosol to subcellular compartments of the plant cell, e.g., mitochondria, chloroplasts, or apoplast/cell wall.

In an embodiment, the signal peptide is selected from the group consisting of barley aleurain vacuole-targeting signal (ALE), Arabidopsis 2S2, Arabidopsis β-expansin, barley α-amylase signal sequence (BAASS), tobacco calreticulin signal peptide (CALSP), Chitinase 1, maize expansin B, leader peptide derived from murine monoclonal antibody mAb24 (MMA), potato protease inhibitor II (PPI), PR-S/Pr1a/Pr1b, PttCel9B3, Phaseolus vulgaris polygalacturonase-inhibiting protein (PvPGIP1), rice α-amylase (−/3A/3D), and fasciclin-like arabinogalactan (FLA) signal peptides.

In an embodiment, the nucleotide secretion signal is BAASS or a FLA signal peptide.

The FLA signal peptide may be derived from any suitable FLA protein known to persons skilled in the art, illustrative examples of which include cotton FLA1, FLA7, FLA9, FLA11, FLA12, FLA17, and the FLA proteins characterized by, e.g., MacMillan et al., (2010, The Plant Journal, 62(4): 689-703), Huang et al. (2008, Physiologia Plantarum, 134: 348) and Guerriero et al. (2017, BMC Genomics, 18: 741).

In an embodiment, the FLA signal peptide comprises an amino acid sequence set forth as SEQ ID NOs: 26 and 28, or an amino acid sequence having at least 90% identity to any of the foregoing.

In an embodiment, the anchoring domain is a carbohydrate binding domain.

In an embodiment, the carbohydrate binding domain is a cellulose binding domain. Suitable cellulose binding domains would be known to persons skilled in the art, illustrative examples of which include CBD-clos (Clostridium thermocellum), CBD-P (Phytophthora infestans), CBD-Cex (Cellumonas fimi), pepC, pepD and TUPpep.

In an embodiment, the cellulose binding domain is CBD-clos (Clostridium thermocellum).

In an embodiment, the nucleic acid construct comprises a nucleotide sequence selected from the group consisting of SEQ ID NOs: 1, 3, 5, 7, 9, 11 and a sequence having 90% identity to any of the foregoing.

In another aspect, the present disclosure provides an isolated nucleic acid molecule comprising a nucleotide sequence encoding one or more pigment producing genes, or a functional fragment or variant thereof, or a complementary form thereof, wherein expression of the nucleic acid sequence is controlled by a fibre-specific promoter, said nucleotide sequence selected from the group consisting of:
(a) a nucleotide sequence having at least 90% identity to the nucleotide sequence set forth in SEQ ID NO: 31 or 32;
(b) a nucleotide sequence comprising the nucleotide sequence set forth in SEQ ID NO: 31 or 32;
(c) a nucleotide sequence having at least 90% identity to the nucleotide sequence set forth in SEQ ID NO: 16 or 17;
(d) a nucleotide sequence comprising the nucleotide sequence set forth in SEQ ID NO:16 or 17;
(e) a nucleotide sequence having at least 90% identity to the nucleotide sequence set forth in SEQ ID NO: 9;
(d) a nucleotide sequence comprising the nucleotide sequence set forth in SEQ ID NO: 9;
(e) a nucleotide sequence having at least 90% identity to the nucleotide sequence set forth in SEQ ID NO: 19 or 20; and
(f) a nucleotide sequence comprising the nucleotide sequence set forth in SEQ ID NO: 19 or 20.

The terms “identity” or “sequence identity” are used interchangeably herein to refer to the extent that the sequences are identical on a nucleotide-by-nucleotide basis or an amino acid-by-amino acid basis over a window of comparison. Thus, a “percentage of sequence identity” is calculated by comparing two optimally aligned sequences over a window of comparison, determining the number of positions at which the identical nucleic acid base (e.g., A, G, T, C and U) or the identical amino acid residue (e.g., Ala, Pro, Ser, Thr, Gly, Val, Leu, Ile, Phe, Tyr, Trp, Lys, Arg, His, Asp, Glu, Asn, Gln, Cyc and Met) occurs in both sequences to yield the number of matched positions, dividing the number of matched positions by the total number of positions in the window of comparison, and multiplying the result by 100 to yield the percentage of sequence identity.

Methods for the determination of nucleic acid sequence identity would be known to persons skilled in the art, illustrative examples of which include computer programs that employ algorithms, such as BLAST (Atschul et al., 1990, Journal of Molecular Biology, 215(3): 403-410).

Nucleotide or amino acid sequences are indicated as “essentially similar” when such sequences have a sequence identity of at least 90% (e.g., at least 90%, 91%, 92%, 93%, 94%, 95%, 96%, 97%, 98%, or 99%). It is clear that when RNA sequences are described as essentially similar to, or have a certain degree of sequence identity with, DNA sequences, thymine (T) in the DNA sequence is considered equivalent to uracil (U) in the RNA sequence.

With regard to the defined nucleic acid molecules, it will be appreciated that % identity figures higher than those provided above will encompass preferred embodiments, Thus, where applicable, in light of the minimum % identity figures, it is preferred that the heterologous nucleic acid molecule comprises a nucleotide sequence which is at least 90%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or more preferably 100% identical to the relevant nominated SEQ ID NO.

### Vectors, Delivery Vehicles and Cells

The present disclosure also provides vectors comprising the nucleic acid construct described herein.

By “vector” it is meant a nucleic acid molecule, preferably a DNA molecule derived from, e.g., a plasmid, bacteriophage, or plant virus, into which a nucleotide sequence may be inserted or cloned. A vector is preferably a double-stranded DNA and contains one or more unique restriction sites and may be capable of autonomous replication in a defined host cell including a target cell or tissue or a progenitor cell or tissue thereof, or capable of integration into the genome of the defined host such that the clone sequence is reproducible. Accordingly, the vector may be an episomal vector (i.e., that does not integrate into the genome of a host cell), or can be a vector that integrate into a host cell genome. Vectors may be replication competent or replication-deficient. Exemplary vectors include, but are not limited to, plasmids (e.g., linear or closed circular plasmids), an extrachromosomal element, a minichromosome, an artificial chromosome and plant viral vectors. The choice and design of an appropriate vector is within the ability and discretion of one of ordinary skill in the art.

In an embodiment, the vector is a plasmid.

Plasmid vectors typically include additional nucleotide sequences that provide for easy selection, amplification, and transformation of the expression cassette in prokaryotic and eukaryotic cells, e.g., pUS-derived vectors, pSK-derived vectors, pGEM-derived vectors, pSP-derived vectors, pBS-derived vectors, or binary vectors containing one or more T-DNA regions. Additional nucleotide sequences include origins of replication to provide for autonomous replication of the vector, selectable marker genes, preferably encoding antibiotic or herbicide resistance, unique multiple cloning sites providing for multiple sites to insert a nucleic acid construct or genes encoded in a nucleic acid construct, and sequences that enhance transformation of prokaryotic and eukaryotic (especially plant) cells.

By “marker gene” it is meant a gene that imparts a distinct phenotype to cells expressing the marker gene and thus allows such transformed cells to be distinguished from cells that do not have the marker. A selectable marker gene confers a trait for which one can “select” bases on resistance to a selective agent (e.g., a herbicide, antibiotic, radiation, heat or other treatment damaging to untransformed cells). Suitable selectable marker genes for the selection of plant transformants would be known to persons skilled in the art, illustrative examples of which include a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione; a glutamine synthetase gene conferring, upon overexpression, resistance to glutamine synthetase inhibitors such as phosphinothricin, an acetyltransferase gene from Streptomyces viridochromogenes conferring resistance to the selective agent phosphinothricin, a gene encoding a 5-enolshikimate-3-phosphate synthase (EPSPS) conferring tolerance to N-phosphonomethylglycine, a bar gene conferring resistance against bialaphos, a nitrilase gene such as bxn from Klebsiella ozaenae which confers resistance to bromoxynil, a dihydrofolate reductase (DHFR) gene conferring resistance to methotrexate, a mutant acetolactate synthase gene (ALS), which confers resistance to imidazolinone, sulfonylurea or other ALS-inhibiting chemicals, or a mutated anthranilate synthase gene that confers resistance to 5-methyl tryptophan.

In an embodiment, the vector comprises a nucleotide sequence set forth as SEQ ID NOs: 33 to 37, or a nucleotide sequence having at least 90% identity to any of the foregoing.

In another aspect, the present disclosure provides a cell, or cell extract derived therefrom, comprising the nucleic acid construct or the vector described herein.

In an embodiment, the cell is a plant cell. Suitable plant cells would be known to persons skilled in the art, illustrative examples of which include cotton, flax, hemp, jute, ramie, kenaf, sisal, banana, manila hemp, pineapple and coir plant cells.

In an embodiment, the cell is a cotton plant cell.

In an embodiment, the cotton plant cell is a Gossypium hirsutum, Gossypium barbadense, Gossypium herbaceum or Gossypium arboretum plant cell.

In an embodiment, the cell is a Gossypium hirsutum plant cell. In another embodiment, the cell is a Gossypium hirsutum variety Coker 315 plant cell.

The cell or cell extract may be provided with the nucleic acid construct or vectors described herein using any suitable method known in the art. Such methods include transfection, transduction, viral transduction, microinjection, lipofection, nucleofection, nanoparticle bombardment, transformation, conjugation and the like. The skilled person would readily understand and adapt and such method taking into consideration whether the nucleic acid construct was provided as vector, e.g., a plasmid or viral vector.

### Polypeptides

In another aspect, the present disclosure provides a polypeptide encoded by the nucleic acid construct described herein.

The terms “polypeptide” and “protein” are generally used interchangeably herein and are intended to include variants, mutants, modifications and/or derivatives of the pigment producing proteins described herein. As used herein, “substantially purified polypeptide” refers to a polypeptide that has been separated from the lipids, nucleic acids, other peptides and other molecules with which it is naturally associated in its native state. Preferably, the substantially purified polypeptide is at least 60% free, more preferably at least 75% free, and more preferably at least 90% free from other components with which it is naturally associated. By “recombinant polypeptide” it is meant a polypeptide made using recombinant techniques, e.g., through the expression of a recombinant polynucleotide in a cell, preferably a plant cell, e.g., a cotton plant cell.

### Methods for Producing a Colored Plant Fibre

In another aspect of the present disclosure, there is provided method of producing colored plant fibre, the method comprising: (i) transforming a plant cell with a the nucleic acid construct or vector described herein; (ii) regenerating a plant from the transformed cell, and (iii) harvesting the fibre from the regenerated plant.

The term “transformation” means alteration of the genotype of an organism, for example a bacterium or a plant, by the introduction of a foreign or exogenous nucleic acid. By “transformant” is meant an organism so altered.

The term “regeneration” as used herein in relation to plant materials means growing a whole, differentiated plant from a plant cell, a group of plant cells, a plant part such as, for example, from an embryo, scutellum, protoplast, callus, or other tissue, but not including growth of a plant from a seed.

The particular choice of a transformation technology will be determined by its efficiency to transform certain plant species as well as the experience and preference of the person practicing the invention with a particular methodology of choice. It will be apparent to the skilled person that the particular choice of a transformation system to introduce a nucleic acid construct into plant cells is not essential to or a limitation of the invention, provided it achieves an acceptable level of nucleic acid transfer. Guidance in the practical implementation of transformation systems for plant improvement is provided by Birch (1997, Annual Review of Plant Physiology and Plant Molecular Biology, 48: 297-326.

Introduction and expression of foreign or exogenous polynucleotides in dicotyledonous plants such as cotton, has been shown to be possible using the T-DNA of the tumor-inducing (Ti) plasmid of Agrobacterium tumefaciens (see, e.g., U.S. Pat. No. 5,004,863). In an embodiment, the heterologous nucleic acid molecule described herein may be introduced into a plant cell utilizing A. tumefaciens containing the Ti plasmid. In using an A. tumefaciens culture as a transformation vehicle, it is most advantageous to use a non-oncogenic strain of the Agrobacterium as the vector carrier so that normal non-oncogenic differentiation of the transformed tissues is possible. It is preferred that the Agrobacterium harbors a binary Ti plasmid system. Such a binary system comprises: (i) a first Ti plasmid having a virulence region essential for the introduction of transfer DNA (T-DNA) into plants, and (ii) a chimeric plasmid. The chimeric plasmid contains at least one border region of the T-DNA region of a wild-type Ti plasmid flanking the nucleic acid molecule to be transferred. Binary Ti plasmid systems have been shown effective to transform plant cells as described by, e.g., De Framond, 1983, Biotechnology, 1: 262; and Hoekema et al., 1983, Nature, 303: 179. Such a binary system is preferred inter alia because it does not require integration into the Ti plasmid in Agrobacterium.

Methods involving the use of Agrobacterium include, but are not limited to transformation of plant cells or tissues with Agrobacterium such as transformation of seeds, apices or meristems with Agrobacterium, or inoculation in planta such as the floral-dip method as described by Clough and Bent, 1998, The Plant Journal, 16(6): 735-743. This approach is based on the infiltration of a suspension of Agrobacterium cells. Alternatively, the nucleic acid construct may be introduced using root-inducing (Ri) plasmids of Agrobacterium as vectors.

Methods for transformation of plants by introduction of an exogenous nucleic acid and for regeneration of plants from cells by somatic embryogenesis are well known in the art. Other methods for introducing the nucleic acid construct into a plant cell are by electroporation, or high velocity ballistic penetration by small particles (also known as particle bombardment or microprojectile bombardment) with the nucleic acid to be introduced contained either within the matrix of small beads or particles, or on the surface thereof as described by, e.g., Klein et al., 1987, Nature, 327: 70.

In an embodiment, the plant cell is a cotton plant cell, a flax plant cell, a hemp plant cell, a jute plant cell, a ramie plant cell, a kenaf plant cell, a sisal plant cell, a banana plant cell, a manila hemp plant cell, a pineapple plant cell or a coir plant cell.

In an embodiment, the plant cell is a cotton plant cell.

In an embodiment, the cotton plant cell is a Gossypium hirsutum, Gossypium barbadense, Gossypium herbaceum or Gossypium arboretum plant cell.

In an embodiment, the cell is a Gossypium hirsutum plant cell. In another embodiment, the cell is a Gossypium hirsutum variety Coker 315 plant cell.

In an embodiment, the method further comprises the step of crossing the plant regenerated in step (ii) with a second plant to produce a progeny plant.

In an embodiment, the second plant is of a different genetic background to the plant regenerated in step (ii).

In another aspect, there is provided a method of introducing one or more pigment producing genes into a plant, wherein the one or more pigment producing genes are heterologous to the plant, the method comprising:
(i) crossing a first parent plant with a second parent plant, wherein said second plant is a plant that is transgenic for the nucleic acid construct described herein; and
(ii) backcrossing a progeny plant of the cross of step (i) with a plant of the same genotype as the first parent plant to produce a plant with a majority of the genotype of the first parent plant but characterized by the expression of the one or more pigment producing genes in the fibre cells of the plant.

In an embodiment, the method further comprises genotyping the progeny plant for the presence or absence of the one or more pigment producing genes using a method of genotyping a plant, the method comprising: (i) obtaining a sample comprising nucleic acid or protein extracted from the progeny plant, and (ii) detecting in the sample the nucleic acid construct, or the polypeptide described herein.

Any molecular biological technique known in the art that is capable of detecting the nucleic acid construct, or the polypeptide can be used in the methods described herein, illustrative embodiments include the use of nucleic acid amplification, nucleic acid sequencing, nucleic acid hybridization with suitably labelled probes, single-strand conformation analysis (SSCA), denaturing gradient gel electrophoresis (DGGE), heteroduplex analysis (HET), or by immunoassays to detect or quantify the expression of a pigment or enzyme encoded by the transgene (i.e., the one or more pigment producing genes).

In an embodiment, the method further comprises analyzing the optical properties of the fibres of the progeny plant for the presence or absence of one or more exogenous pigments, the method comprising: (i) obtaining a sample comprising fibre cells from the progeny plant, and (ii) using a spectroscopic method to detect pigments across visible or non-visible wavelengths.

Suitable spectroscopic methods would be known to persons skilled in the art, illustrative methods include absorbance spectroscopy, fluorescence spectroscopy and infrared spectroscopy. In some embodiments, the spectroscopic method utilizes the excitation and emission wavelengths presented in Table 2.

It is further contemplated herein that the introduction of pigment producing genes that encode or produce pigments on the non-visible spectra may be used for tracing and tracking purposes, e.g., in precision agriculture applications for tracing the harvested cotton lines from the farm and across supply chains.

In an embodiment, the plant is a cotton plant.

### Plants and Products Derived Therefrom

In another aspect of the present disclosure, there is provided a plant modified to produce colored fibre, wherein the plant comprises the nucleic acid construct described herein, and wherein the one or more pigment producing genes are expressed in fibre cells of the plant.

As used herein, the term “plant” refers to whole plants, but as used as an adjective refers to any substance which is present in, obtained from, derived from, or related to a plant, including plant organs (e.g., leaves, stems, roots, flowers), single cells (e.g., pollen), seeds and plant cells. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (e.g., leaves, stems), roots, floral organs/structures, seed (e.g., embryo, cotyledons, seed coat), plant tissue (e.g., vascular tissue, ground tissue), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, e.g., calli. Exemplary plant tissues in or from seeds are cotyledon, embryo and embryo axis.

In an embodiment, the plant is a cotton plant, a flax plant, a hemp plant, a jute plant, a ramie plant, a kenaf plant, a sisal plant, a banana plant, a manila hemp plant, a pineapple plant or a coir plant.

In an embodiment, the plant is a cotton plant.

In an embodiment, the plant is a transgenic plant.

The term “transgenic plant” as used herein refers to plants and their progeny, which have been genetically modified using recombinant techniques. This would generally be to produce at least one pigment producing protein described herein in the desired plant or plant organ. Transgenic plant parts include all parts and cells of said plants which comprise the transgene such as, e.g., seeds, cultured tissues, callus and protoplasts. Transgenic plants contain genetic material that they did not contain prior to the transformation. The genetic material is typically stably integrated into the genome of the plant. The introduced genetic material may comprise sequences that naturally occur in the same species but in a rearranged order or in a different arrangement of elements, e.g., an antisense sequence or a sequence encoding a double-stranded RNA. Such plants are included herein in “transgenic plants”. In an embodiment, the transgenic plants are homozygous for each gene that has been introduced (e.g., transgene) so that their progeny do not segregate for the desired phenotype.

Methods for the transformation of plants to introduce an exogenous nucleic acid molecule (e.g., the nucleic acid construct described herein) would be known to persons skilled in the art, illustrative examples of which include acceleration of genetic material coated onto micro particles directly into cells, transformation by Agrobacterium-mediated technology, and electroporation technology. In an embodiment, transgenic plants are produced by Agrobacterium tumefaciens-mediated transformation procedures. Vectors comprising the desired nucleic acid construct may be introduced into regenerable cells of tissue cultured plants or explants, or other suitable plant cells, e.g., protoplasts.

As used herein, the term “corresponding non-transgenic plant” refers to a plant which is the same or similar in most characteristics, preferably isogenic or near-isogenic relative to the transgenic plant, but without the heterologous nucleic acid molecule described herein. Preferably, the corresponding non-transgenic plant is of the same cultivar or variety as the progenitor of the transgenic plant of interest, or a sibling plant line which lacks the genetic modification, often termed a “segregant”, or a plant of the same cultivar or variety transformed with an “empty vector” construct, and may be a non-transgenic plant.

It is also contemplated herein that the nucleic acid construct can be introduced using biologically-based techniques. Suitable biologically-based techniques would be known to persons skilled in the art, illustrative examples of which include the use of biological agents for introducing exogenous nucleic acid sequences, e.g., CRISPR, endonucleases, ZFNs, meganucleases, etc.

As used herein, the term “biological agents” means any agent useful to insert exogenous sequences, which includes enzymes that induce double stranded breaks in DNA that stimulate endogenous repair mechanisms. These include endonucleases, zinc finger nucleases, TAL effector proteins, transposases, site-specific recombinases and are CRISPR endonucleases. Zinc finger nucleases (ZFNs), e.g., facilitate site-specific cleavage within a selected gene within a genome allowing endogenous or other end-joining repair mechanisms to introduce insertions to repair the gap. Zinc finger nuclease technology is described in Le Provost et al. (2009, Trends in Biotechnology, 28(3): 134-141), Durai et al. (2005, Nucleic Acids Research, 33: 5978-5990) and Liu et al. (2010, Biotechnology and Bioengineering, 106: 97-105).

The term “wild-type” as used herein refers to a cell, tissue or plant that has not been modified. Wild-type cells, tissue or plants that are known in the art may be used as controls to compare the levels of expression of endogenous or exogenous nucleic acid molecules or polypeptides, or the extent and nature of trait modification in cells, tissue, or plants modified as described herein. For example, as used herein, “wild-type cotton” means a cotton plant (or part thereof) that has not been modified, e.g., non-transgenic. Specific wild-type cotton suitable for cultivation include, but are not limited to, cotton cultivar Coker 315.

The skilled person will appreciate that where a comparison is made between the plants or fibre of the present disclosure and those which are wild-type, the comparison is performed with plants grown under essentially identical growing conditions, growth time, temperature, water and nutrient supply, etc., and for cotton lint obtained from such plants.

As used herein, the term “progeny” includes all offspring from a plant, both the immediate and subsequent generations, and both plants and seed. Progeny include the seeds and plants obtained after self-fertilization (“selfing”) and the seed and plants resulting from a cross between two parental plants, such as the F1 offspring (first generation), F2, F3, F4, etc., being the offspring from the second etc., generations after selfing of the F1 plants.

The present disclosure also provides a cotton plant modified to produce colored fibre, wherein the cotton plant comprises the nucleic acid construct described herein, and wherein the one or more pigment producing genes are expressed in fibre cells of the cotton plant.

Reference to a “cotton plant” refers to any plant belonging to the genus Gossypium and to any wild relatives, progenitor species, germplasm, cultivar or variety thereof. For example, cotton plants include species such as: G. anapoides, G. anomalum, G. arboreum, G. areysianum, G. aridum, G. armourianum, G. australe, G. barbadense, G. barbosanum, G. benadirense, G. bickii, G. briccetti, G. capitis-viridis, G. costulatum, G. cunningharnii, G. darwinii, G. davidsonii, G. enthyle, G. exiguum, G. gossypioides, G. harknessii, G. herbaceum, G. hirsutum, G. incanum, G. klotzschianum, G. laxum, G. lobatum. G. londonderriense. G. longicalyx G. marchantii. G. mustelinum. G. nandewarense, G. nelsonii, G. nobile, G. pilosum, G. populifoliurn, G. pulchellum, G. raimondii, G. robinsonii, G. rotundifoliurn, G. schwendimantii, G. somalense, G. soudanense, G. stocksii, G. sturtianum, G. thurberi, G. timorense, G. tomentosum, G. trilobum, G. triphyllum, and G. vindis. Particular species are cotton species suitable for cultivation, including Gossypium hirsutum, Gossypium barbadense, Gossypium herbaceum and Gossypium arboretum. Persons skilled in the art will appreciate that one convenient variety, cultivar or mutant may be genetically modified and the phenotype bred into related varieties or species by standard breeding techniques. Reference to “progenitor” refers to any of the species, varieties, cultivars, or germplasm, from which a plant is derived.

As used herein, the term “derivative species, germplasm or variety” shall be taken to mean any plant species, germplasm or variety that is produced using a stated cotton species, variety, cultivar, or germplasm, using standard procedures of sexual hybridization, recombinant DNA technology, tissue culture, or a combination of any one or more said procedures. In particular, interspecific hybrids have been produced between various important cotton species such as cotton species suitable for cultivation, including Gossypium hirsutum, Gossypium barbadense, Gossypium herbaceum and Gossypium arboretum, and between certain diploid species, and the production of such interspecific hybrids is routine to those skilled in the art.

As used herein, the term “cotton” refers to any species of the Genus Gossypium, preferably cotton species suitable for cultivation, including Gossypium hirsutum, Gossypium barbadense, Gossypium herbaceum and/or Gossypium arboretum. In some embodiments, a suitable control or reference plant is a non-transformed substantially isogenic cotton plant treated in the same way as the “test” plant.

In another aspect, the present disclosure provides a method of producing cotton seed, wherein the method comprises crossing the plant described herein with itself, or a second, distinct cotton plant to produce cotton seed.

In an embodiment, the method further comprises: (i) growing a progeny plant from a subsequent generation from the cotton seed and crossing the progeny plant of a subsequent generation with itself or a second plant; and (ii) repeating step (i) for additional generations with sufficient inbreeding to produce seed of an inbred cotton plant.

In another aspect, the present disclosure provides cotton seed produced by the method described herein.

Persons skilled in the art would appreciate that where cotton seed is produced for use in horticultural or crop industries, seed coating may be performed to coat the cotton seed with low amounts of exogenous materials (e.g., growth media, cultivation media, microbial inoculant, fungicide, pesticide), to improve seedling establishment, increase plant yield, nutrition, and tolerance to biotic-abiotic stresses (see, e.g., Azfal et al., 2020, Agriculture, 10: 526). Colorants and dyes may also be used to facilitate traceability, labelling, variety and treatment. Binding agents, and sometimes filling agents are used in a seed coating formulation to assist the adherence of any active ingredient to the seed (Rocha et al., 2019, Frontiers in Plant Science, 10: 1357). Commercially available cotton seed coatings include Vibrance Complete, Genero 600, Cruiser, Avicta, Dynasty CST, Trilex and CruiserExtreme.

Accordingly, in another aspect, the present disclosure provides a composition comprising the cotton seed described herein, wherein the seed is comprised in a plant seed coating, and wherein said seed coating comprises one or more of a fungicide, pesticide, colorant.

Accordingly, in another aspect, the present disclosure provides a composition comprising the cotton seed described herein, wherein the seed is comprised in plant seed growth media, and wherein said plant seed growth media comprises soil or synthetic cultivation media.

The present inventors have shown that the exogenous pigments, such as chromoproteins or betalains, can be produced in the fibre cells of cotton plants by the expression of a nucleic acid construct comprising a nucleotide sequence comprising one or more pigment producing genes, wherein expression of the nucleotide sequence is under the control of a fibre-specific promoter, wherein the fibre-specific promoter is capable of driving expression of the nucleotide sequence during two or more fibre developmental stages. The unexpected technical advantage of the approach described herein is the provision of colored cotton fibre that is produced in planta. The selective expression of the one or more pigment producing genes in fibre cells has been shown to produce cotton plants with colored fibre without causing adverse effects to plant development or fibre quality, relative to wild-type cotton plants. By generating colored cotton fibre in planta, the inventors have beneficially developed a more sustainable and less toxic approach for producing colored cotton fibre, and derivable products (lint, yarn, etc.) as compared to current methods for scouring and dying cotton fibre (or lint or yarn comprising such fibres).

In an embodiment, the exogenous pigment is selected from the group consisting of a chromoprotein, a betalain, an anthocyanin, a carotenoid, an indigoidine, melanin, and combinations thereof.

In an embodiment, the exogenous pigment is selected from the group consisting of eforRED (Echinopora forskaliana), amilGFP (Acropora millepora), amilCP (Acropora millepora) and combinations of the foregoing.

In another embodiment, the exogenous pigment is amilGFP (Acropora millepora). In accordance with this embodiment, the cotton plant is modified to produce red fibre.

In an embodiment, the exogenous pigment is a betalain selected from the group consisting of a betacyanin, a betaxanthin, and combinations of the foregoing.

In another embodiment, the exogenous pigment is betanin. In accordance with this embodiment, the cotton plant is modified to produce pink/purple fibre.

In another embodiment, the exogenous pigment is vulgaxanthin I. In accordance with this embodiment, the cotton plant is modified to produce yellow/orange fibre.

In an embodiment, the exogenous pigment is present in the fibre at ≥40 DPA (e.g., 40, 41, 42, 43, 44, 45, 46, 47, 48, 49, 50, 51, 52, 53, 54, 55, 56, 57, 58, 59, or 60 DPA).

In an embodiment, the exogenous pigment is present in the fibre at ≥50 DPA. In another embodiment, the exogenous pigment is present in the fibre at ≥55 DPA.

In an embodiment, the exogenous pigment is substantially stable in mature fibre.

By “substantially stable” it is meant that exogenous pigment accumulated during fibre development is not degraded.

In an embodiment, the exogenous pigment is substantially stable in the fibre at ≥40 DPA (e.g., 40, 41, 42, 43, 44, 45, 46, 47, 48, 49, 50, 51, 52, 53, 54, 55, 56, 57, 58, 59, or 60 DPA).

In an embodiment, the exogenous pigment is substantially stable in the fibre at ≥50 DPA. In another embodiment, the exogenous pigment is substantially stable in the fibre at ≥55 DPA.

In an aspect disclosed herein, there is provided a method of producing a commodity plant product, wherein the method comprises collecting the commodity plant product from the plants described herein.

In an embodiment, the commodity plant product is lint or fibre.

In another aspect, the present disclosure provides a method of producing the cotton lint described herein, the method comprising: (i) growing the cotton plant described herein; and (ii) harvesting the cotton lint.

In an embodiment, the method further comprises drying the harvested cotton lint.

In an embodiment, the harvested cotton lint is dried using a method selected from the group consisting of convective drying, contact drying, dielectric drying and freeze drying.

In an embodiment, the dried harvested cotton lint has a dry basis moisture content of less than about 7% by weight (e.g., about 7%, about 6%, about 5%, about 4%, about 3%, about 2% or about 1%).

In another embodiment, the harvested cotton lint is dehydrated.

In an embodiment, the method further comprises fixing the harvested cotton line in a solvent comprising alcohol.

In an embodiment, the alcohol is selected from the group consisting of ethanol, propan-2-ol and trichloroethene. In an embodiment, the alcohol is ethanol.

In another aspect, the present disclosure provides cotton lint obtained by the method disclosed herein.

In another aspect, the present disclosure provides cotton lint obtained from the cotton plant described herein.

By “cotton lint” it is meant the fibrous coat that covers cottonseed, which is separated from the cottonseed (and trash) by ginning. Cotton lint comprises “cotton fibre” or “cotton fibre cells”, which are single-celled trichomes that differentiate from the ovule epidermis. Cotton lint is processed into yarn, textiles (e.g., fabric) and other products (e.g., paper and personal hygiene products, such as tampons, bandages, cotton buds and cotton balls).

Cotton fibre fineness is defined in terms of linear density (e.g., milligrams/kilometer; “millitex” or “mTex”), with commercial cotton fibre falling within the range of from about 160 mTex to about 240 mTex.

Accordingly, in an embodiment, the cotton lint comprises fibres with an average fineness of from about 160 mTex to about 240 mTex. In another embodiment, the cotton lint comprises fibres with an average fineness of from about 180 mTex to about 215 mTex.

Fibre maturity is defined as the relative thickening of the fibre cell wall. The theoretical range of maturity ratio is from 0.2 to 1.2, with immature fibres having an average maturity ratio of <0.8 and mature fibres having an average maturity ratio of from about 0.8 to about 1.0. Fibres having a maturity ratio of >1 are considered to be very mature.

In an embodiment, the cotton lint comprises fibres with an average maturity ratio of ≥0.8 (e.g., 0.8, 0.9, 1.0, 1.1 and 1.2).

Methods for the measurement of fibre fineness and maturity ratio would be known to persons skilled in the art, illustrative examples of which include the use of the CottonScope instrument, as described elsewhere herein.

In another aspect, the present disclosure provides yarn comprising the cotton lint described herein.

In an embodiment, the yarn further comprises one or more additional fibres selected from the group consisting of polyester, viscose, acrylic, wool and nylon.

Where cotton lint (i.e., fibre) is combined with one or more additional fibres, the resulting yarn is referred to as a “blended yarn”. The ratio of cotton to the one or more additional fibres will be dependent on the application of the blended yarn, e.g., textile, medicinal uses, etc. Suitable ratios of cotton to the one or more additional fibres would be known to persons skilled in the art, illustrative examples of which include cotton and polyester blends of 65/35, 67/33, 70/30, 50/50, 45/55 and 52/48 (polyester/cotton); cotton and acrylic blends of 75/25, 60/40 and 50/50 (cotton/acrylic); cotton and viscose blends of 55/45, 65/35, 70/30 and 85/15 (cotton/viscose). Binary blended yarns (i.e., comprising two fibre components) and tertiary blended yarns (i.e., comprising three or more fibre components) are contemplated herein.

In an embodiment, the yarn (i.e., the blended yarn) comprises at least about 30% cotton (e.g., 30%, 40%, 50%, 60%, 70%, 80%, 90% cotton).

Accordingly, in an embodiment, the yarn comprises at least about 30% cotton, preferably at least about 31%, at least about 32%, at least about 33%, at least about 34%, at least about 35%, at least about 36%, at least about 37%, at least about 38%, at least about 39%, at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, or more preferably at least about 95% cotton.

In another aspect, the present disclosure provides a method of producing yarn, the method comprising: (i) obtaining the cotton lint described herein; and (ii) spinning the cotton lint to produce the yarn.

In an embodiment, the method further comprises drying the cotton lint before step (ii).

In an embodiment, the method further comprises fixing the cotton lint in a solvent comprising alcohol before step (ii).

In an embodiment, step (i) further comprises harvesting the cotton lint from a cotton plant and/or ginning the cottonseed from the lint; and/or wherein step (ii) comprises one or more or all of carding, drawing, combing and roving before spinning the cotton lint to produce the yarn.

In an embodiment, step (i) further comprises obtaining one or more additional fibres, and wherein step (ii) further comprises spinning the cotton lint and the one or more additional fibres to produce a blended yarn.

In an embodiment, wherein the one or more additional fibres is selected from the group consisting of polyester, viscose, acrylic, wool and nylon.

In an embodiment, wherein the blended yarn comprises at least about 30% cotton.

In an aspect, the present disclosure provides yarn produced by the methods disclosed herein.

In an aspect, the present disclosure provides the use of the cotton lint or the yarn described herein to produce a textile.

The term “textile” as used herein refers to any material comprising interlacing fibres (e.g., any woven or knitted fabric) for any use. Suitable textiles would be known to persons skilled in the art, illustrative examples of which include fabric, geotextiles, medical dressings, bandages, carpet, denim, linen, technical textiles, and the like.

In another aspect, the present disclosure provides a textile comprising the yarn described herein.

In an aspect, the present disclosure provides a method to produce a textile, the method comprising: (i) obtaining the yarn described herein; and (ii) weaving or knitting the yarn into a fabric to produce the textile.

All publications mentioned in this specification are herein incorporated by reference. The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavor to which this specification relates.

It will be appreciated by persons skilled in the art that numerous variations and/or modifications may be made to the present disclosure without departing from the spirit or scope of the disclosure as broadly described. The present embodiments are, therefore, to be considered in all respects as illustrative and not restrictive.

The present disclosure will now be further described in greater detail by reference to the following specific examples, which should not be construed as in any way limiting the scope of the disclosure.

### EXAMPLES

### Example 1—Materials and Methods

### Constructs

### Betalains

Four genes from the betalain synthesis pathway: cDOPA5GT (GenBank ID: AB182643.1; SEQ ID NO: 1), DODA1 (GenBank ID: HQ656027.1; SEQ ID NO: 3), CYP76AD1 (GenBank ID: HQ656023.1; SEQ ID NO: 5) and CYP76AD6 (GenBank ID: KT962274.1; SEQ ID NO: 7) were used in this study. The coding sequences (CDS) of those genes were codon optimized for Arabidopsis and synthesized by GeneArt (Thermo Fisher Scientific). Golden Gate SC linker sequences and protect linkers were added and the Golden Gate cloning method was used to assemble different genes into one construct as previously described (Engler et al., 2008, PLoS One, 3(11): e3647). Three genes (DODA1, CYP76AD1 and cDOPA5GT) were assembled into a single construct to produce purple betanin. Two genes (DODA1 and CYP76AD6) were assembled into a single construct to produce yellow/orange betaxanthins (Table 1). The constitutive promoter 2×35S (SEQ ID NO: 14) or the cotton fibre-specific promoter ltp3/8K12 (SEQ ID NO: 16) were used to drive the expression of the gene constructs within the pAGM4723 vector. A kanamycin resistance gene was included in the vectors under the control of a 2×35S constitutive promoter.

### Chromoproteins

The CDS of chromoprotein amilGFP (GenBank ID: AY646067.1; SEQ ID NO: 9) was codon optimized for Arabidopsis and synthesized through GeneArt with the Gateway cloning adaptors at 3′-attL1 and 5′-attL2 ends. The synthesized sequence was then cloned into pMDC32 vector by the LR gateway reaction with the 2×35S promoter and the plant selectable marker gene for hygromycin resistance.

Nicotiana benthamiana Leaf Infiltration

Agrobacterium tumefaciens GV3101 was used for Nicotiana benthamiana leaf infiltration as described by Stoddard and Rolland (2019, Plant Direct, 3(1): e00112). Agrobacteria transformed with plasmids of interests were grown in LB median with rifampicin (50 μg/ml) and either kanamycin (25 μg/ml) or hygromycin (25 μg/ml) at 28° C. in dark with shaking (around 200 rpm) for 24 hours.

The P19 protein of the tomato bushy stunt virus can inhibit post-transcriptional gene silencing, which enables the expression of other constructs (Roth et al., 2004, Virus Research, 102(1): 97-108). Agrobacteria transformed with the vector encoding P19 (OD600=0.3) were mixed with Agrobacteria transformed with the plasmid of interest (OD600=0.5) for infiltration. Agrobacteria transformed with the vector encoding P19 alone were used as the negative control. Cells were centrifuged for 8 minutes at 2150 rpm and the supernatant was removed. Cells were then re-suspended in 10 mM MES pH 5.6, 10 mM MgCl₂, and 150 μM acetosyringone and incubated for 2 hours at room temperature. The abaxial side of expanding Nicotiana benthamiana leaves were infiltrated using a 1 mL syringe.

### Tissue Culture Transformation

Coker 315-11 derived from a single plant selection of cotton cv. Coker 315 was used as the recipient of transformation by infecting the Agrobacterium tumefaciens strain (AGL1 or GV3101) with the constructs described above in accordance with the method of Murray et al. (1999, Molecular Breeding, 5(3): 219-232). Following transformation with the 2×35S promoter driven construct, Coker 315 derived embryogenic callus were grown on selection media up to plantlet. The developing plantlet was then moved into taller pots with rooting media to form a more established plant.

### Plant Growth

Nicotiana benthamiana seeds were sown in soil and plants were grown in a CONVIRON growth cabinet for 5 weeks under the following conditions: 16 hour/8 hour day/night photoperiod, 23° C. and 100 μmol photons m⁻² s⁻¹ of light intensity.

Transgenic cotton plants and wild-type cotton plants were grown in greenhouse at 28° C./20° C. day/night temperatures with natural light. Osmocote Exact Standard (ICL Specialty Fertilizers) fertilizer was added regularly, and plants were watered by an automatic watering system.

### Freeze Drying

Cotton flowers were tagged on the day they open as 0 Days Post Anthesis (DPA). Cotton bolls were collected at around 10, 15, 20, 25, 30, 40, 50 and 60 DPA. The boll coats were either cut open or removed entirely before 48 hours of freeze drying.

### Confocal Imaging

A Leica SP8 confocal laser-scanning microscope (Leica Microsystems), which is manipulated through the Leica LASX software, was used to image various plant samples, including infiltrated Nicotiana benthamiana leaves, transgenic cotton leaves and fibres at different developmental stages. The 10× (NA=0.3), 20× (NA=0.5) or 40× (NA=1.1) water immersion objectives were used. Fresh plant tissue samples (leaf discs or fibres) were mounted in water and imaged with the confocal laser-scanning microscope. For the imaging of plant samples expressing amilGFP, the excitation was set at 503 nm, and the emission was set at 508-550 nm. For the imaging of plant samples expressing betalain, the excitation was set at 540 nm, and the emission was set at 620-640 nm. For the imaging of chloroplasts, the emission was set at 650-690 nm.

### Fibre Quality Assessment

Fineness and maturity ratio of fibres obtained from the transgenic cotton plants described herein was measured using the Cottonscope® (BSC Electronics Pty Ltd, WA, Aus). The Cottonscope® is a fully automated microscope that measures cotton maturity, width and fineness, as previously described by Rodgers et al (2012, Textile Research Journal, 82(3): 259-271). The Cottonscope® was used according to the manufacturer's instructions.

### Example 2—Recombinant Expression of Betalain Pathway Genes

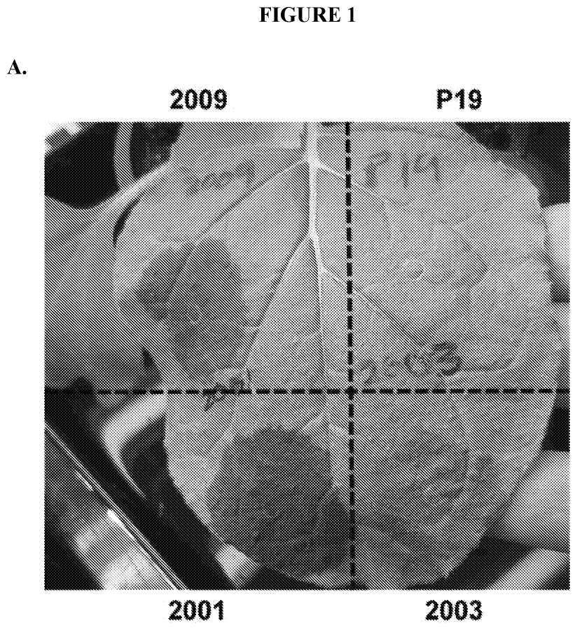

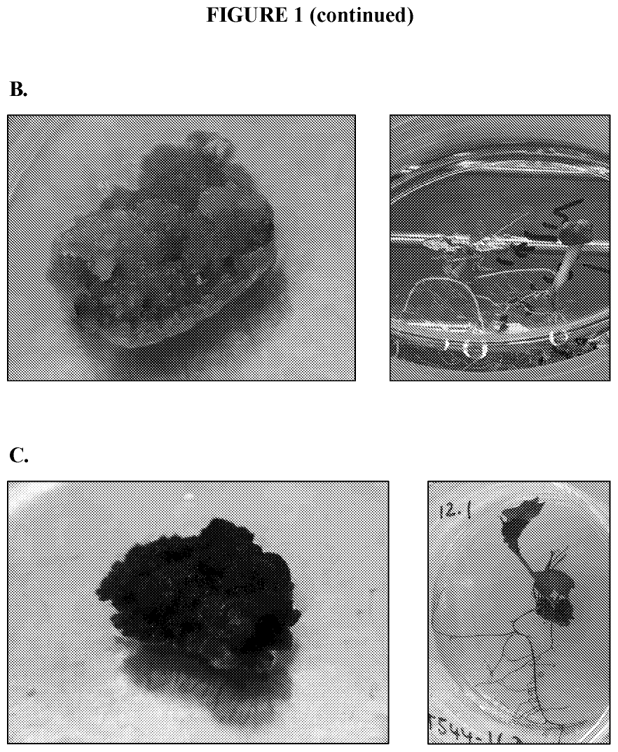

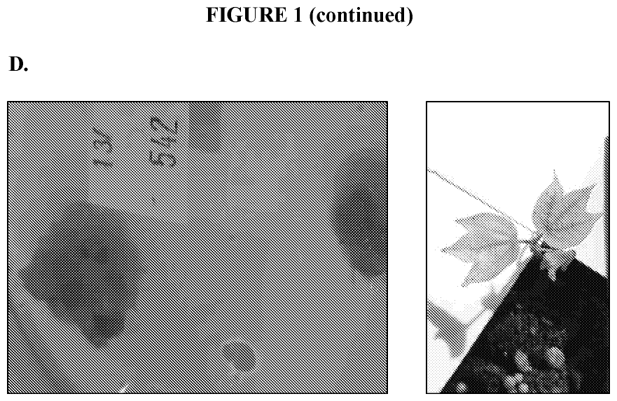

Constructs for the heterologous expression of betalain pathway genes were transiently expressed in Nicotiana benthamiana leaves following infiltration of the 2001 vector comprising a construct of DODA1, CYP76AD1 and cDOPA5GT (i.e., the purple pigment construct) driven by the 2×35S constitutive promoter, the 2003 vector comprising a construct of DODA1 and CYP76AD6 (i.e., the yellow/orange pigment construct) driven by the 2×35S constitutive promoter and the 2009 vector comprising the purple pigment construct driven by the fibre-specific promoter to generate purple or yellow/orange pigments (FIG. 1A). The 2×35S constitutive promoter successfully activated the betalain pathway genes for both the 2001 and 2003 vectors. Similarly, the fibre-specific promoter was able to activate the betalain pathway genes for the 2009 vector.

The same vectors were stably transformed into cotton using the tissue culture methods described elsewhere herein. Compared to the callus transformed with the control P19 vectors (FIG. 1B), the callus transformed with the 2001 vector and the resulting plants/plantlets generated purple pigment (FIG. 1C) and the callus transformed with the 2003 vector and the resulting plants/plantlets generated yellow/orange pigment (FIG. 1D).

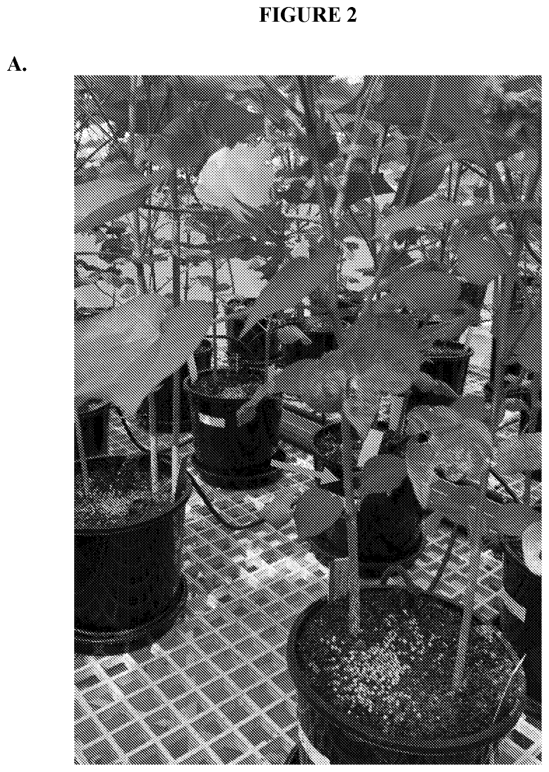

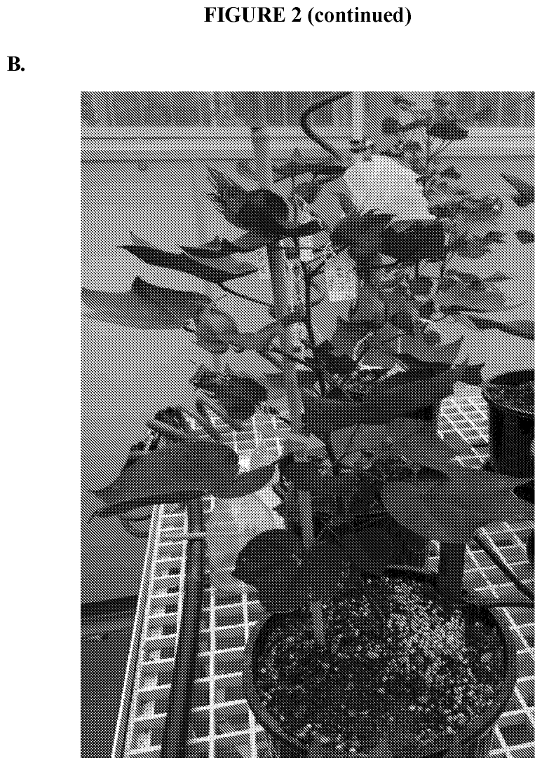

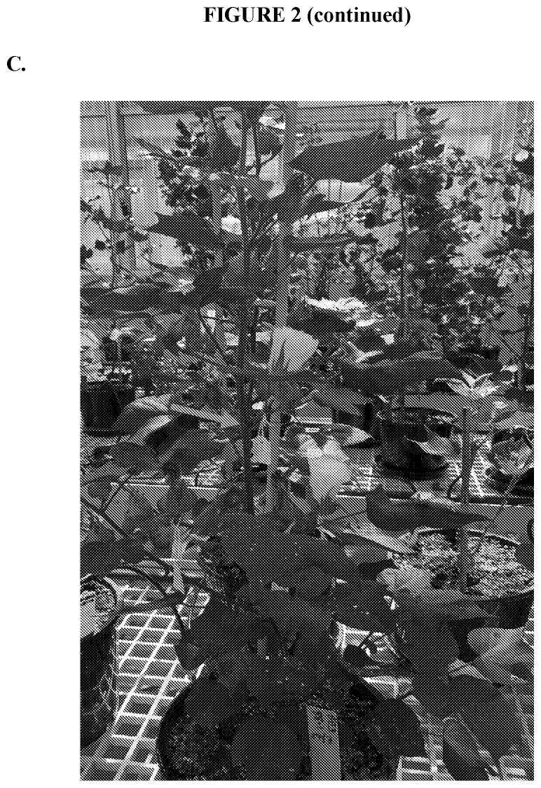

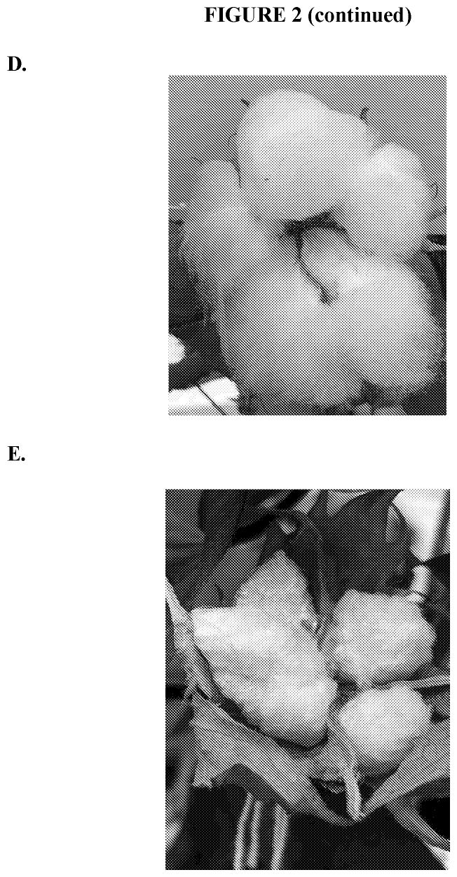

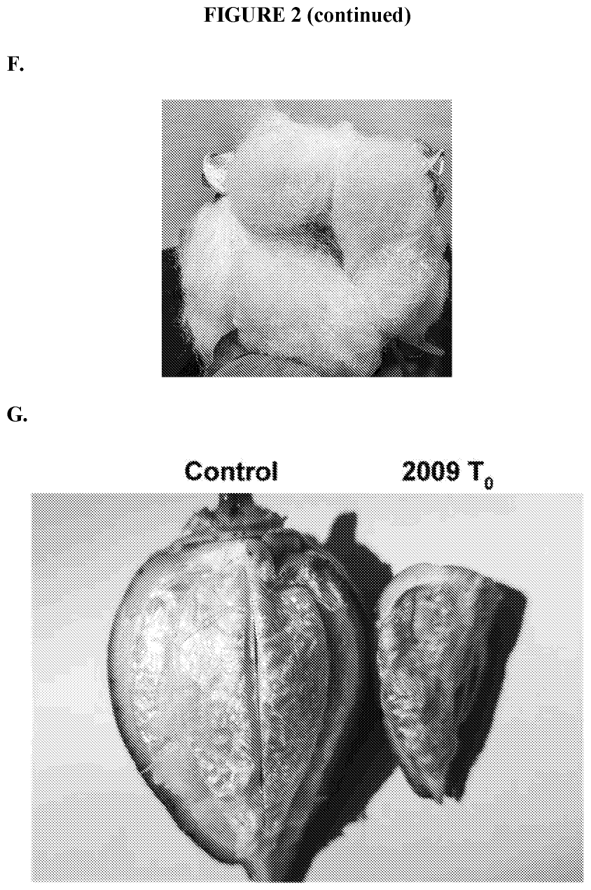

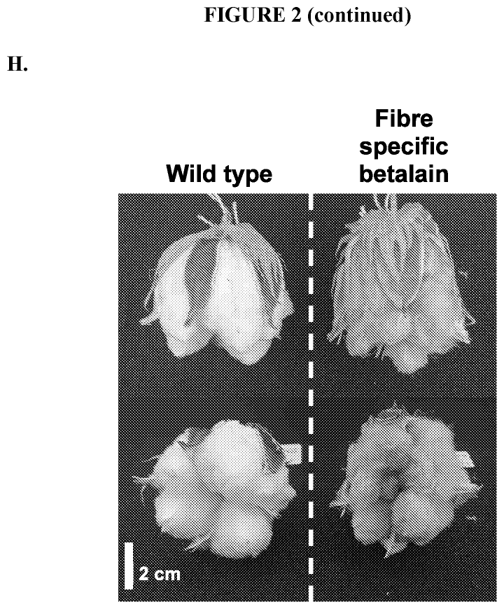

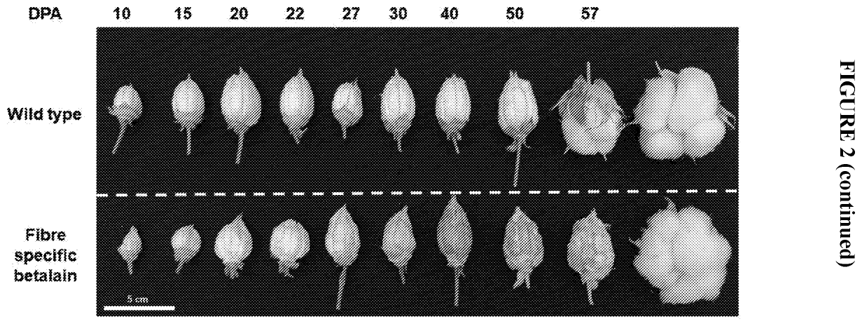

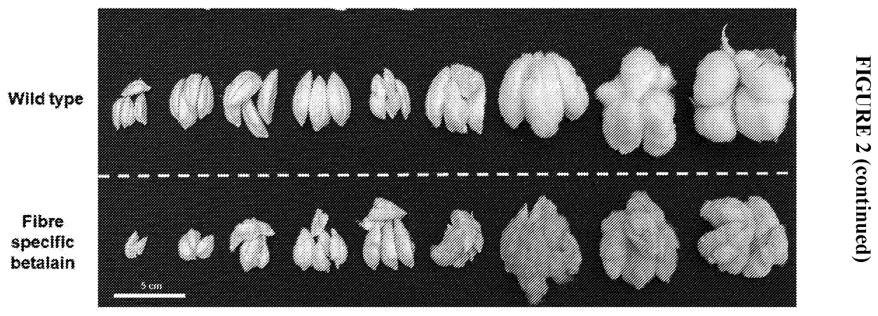

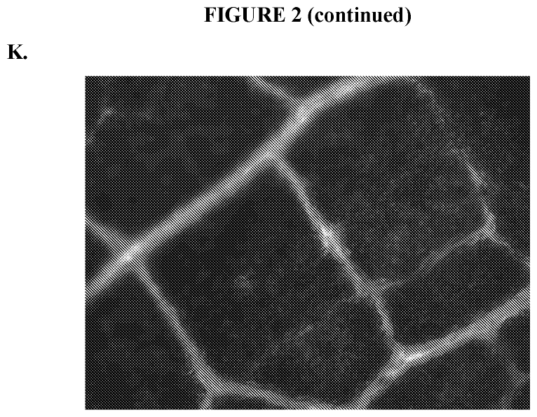

Transgenic cotton plants stably expressing the 2001 vector produced purple pigment throughout the entire plant and pink flowers at 0 days post-anthesis (DPA), as compared to control plants that have green foliage with creamy yellow flowers (FIGS. 2A and 2B). Transgenic cotton plants stably expressing the 2009 vector produced green foliage or partially purple foliage with creamy yellow flowers consistent with the control plants at 0 DPA (FIG. 2C). The control cotton boll had white fibres (FIG. 2E), whereas the cotton boll of the transgenic cotton plants stably expressing the 2009 vector with the fibre-specific promoter produced pink fibres before drying, and light brown fibres when dried and opened (FIG. 2G).

### Example 3—Recombinant Expression of Chromoprotein Genes

A construct for the heterologous expression of amilGFP was transiently expressed in Nicotiana benthamiana leaves following infiltration (FIG. 3A). While there was no color change observable between the leaves infiltrated with the amilGFP construct and the control leaves, the fluorescence signal of amilGFP was detected with infiltrated Nicotiana benthamiana leaf (FIG. 3A).

T₀ transgenic cotton plants transduced with the same constructs had detectable amilGFP fluorescence signal in both leaves and developing fibres (FIGS. 3B and 3C). Expression of amilGFP was observed using confocal microscopy in the cytosol and immature cotton fibres following transduction with constructs in the absence of a nucleotide sequence encoding a signal peptide. Further, transduction of constructs encoding a signal peptide resulted in amilGFP expression in the secondary cell wall (FIGS. 3C and 3D).

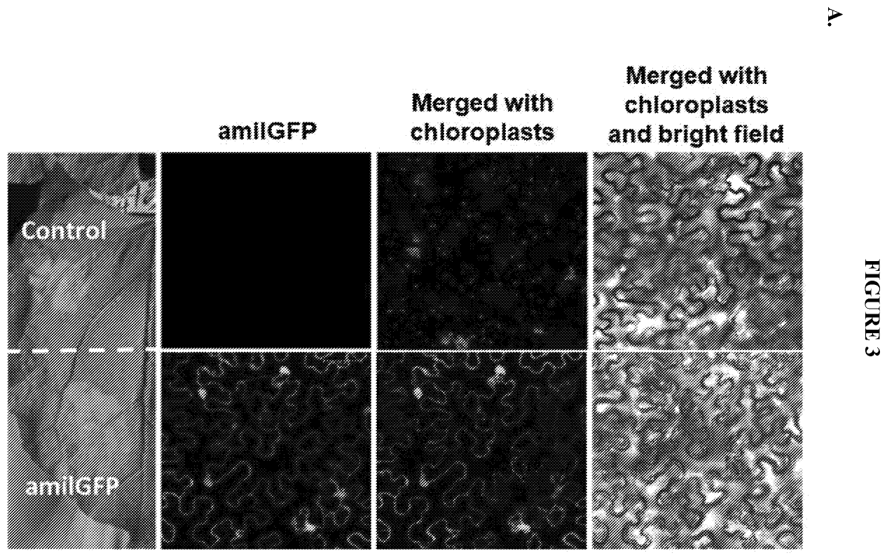

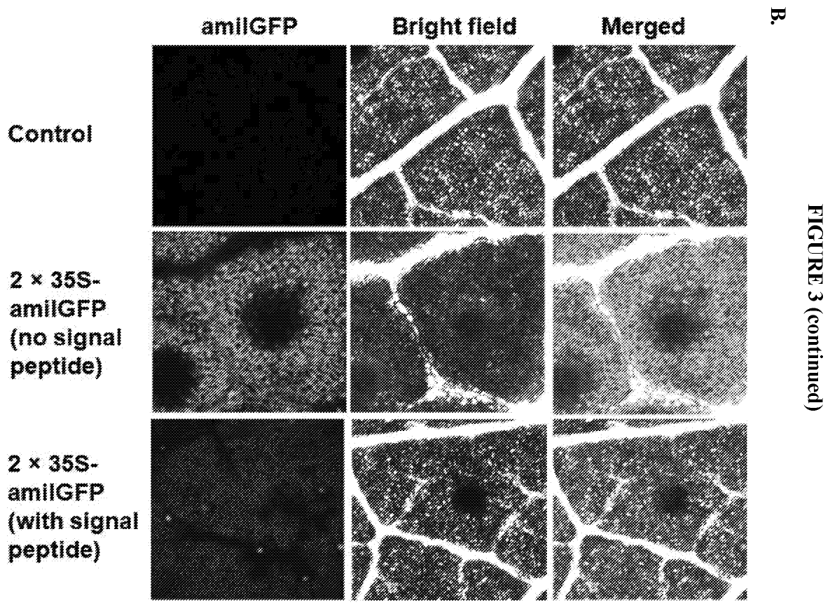

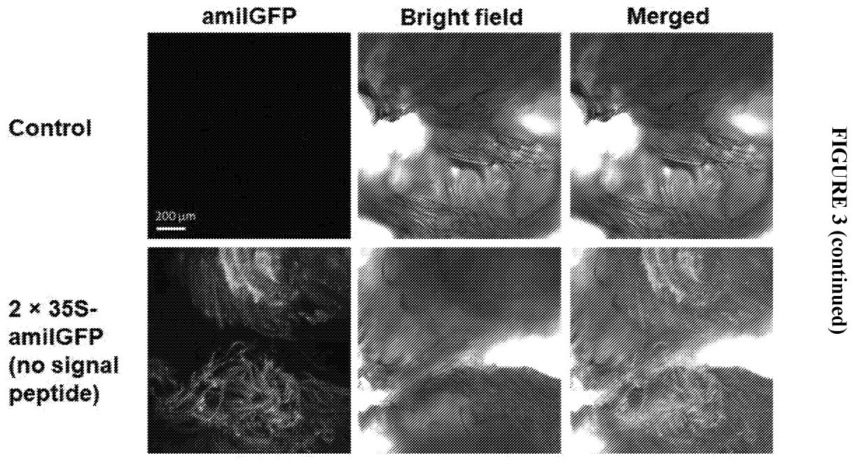

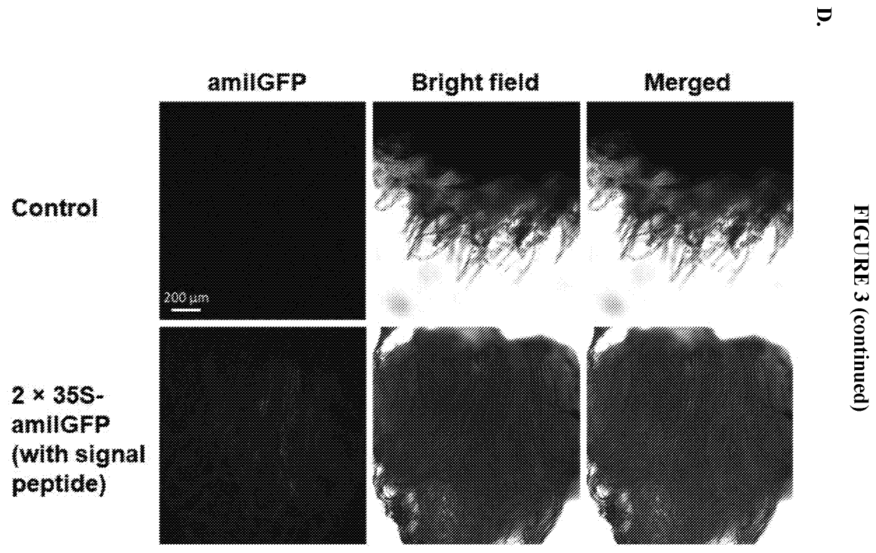

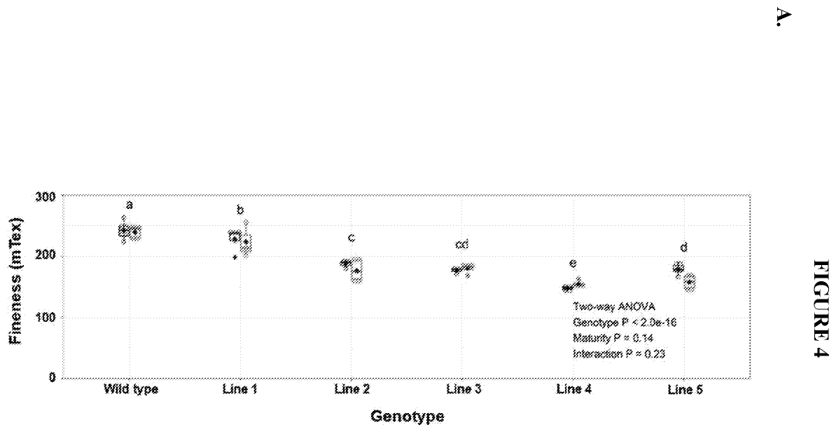

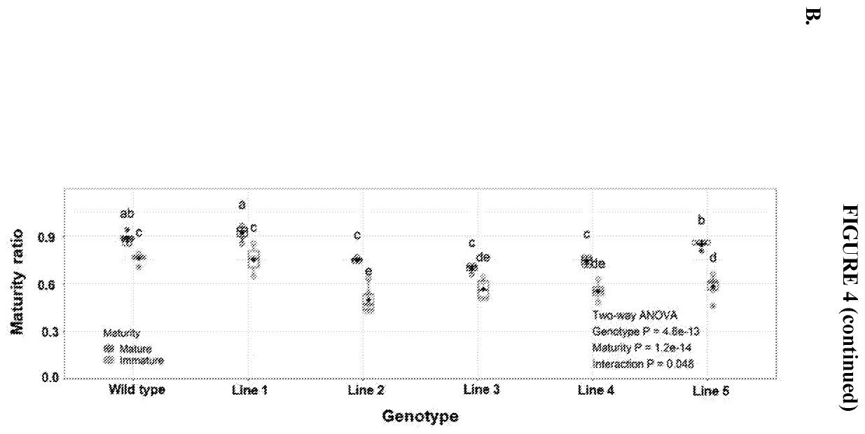

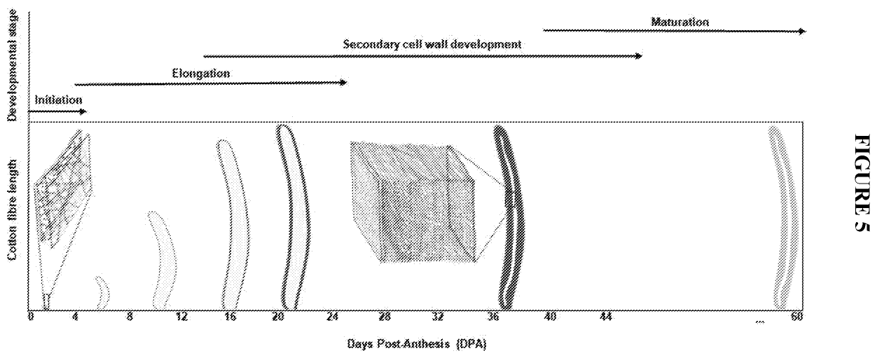

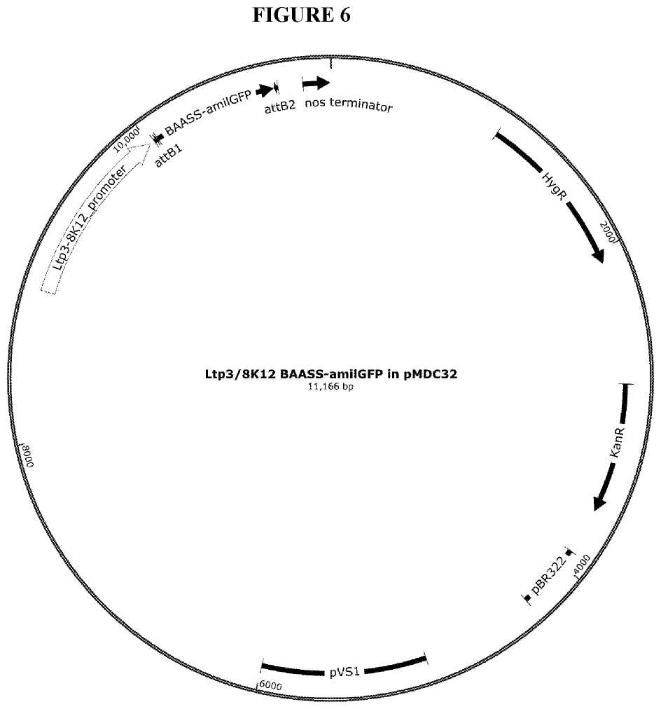

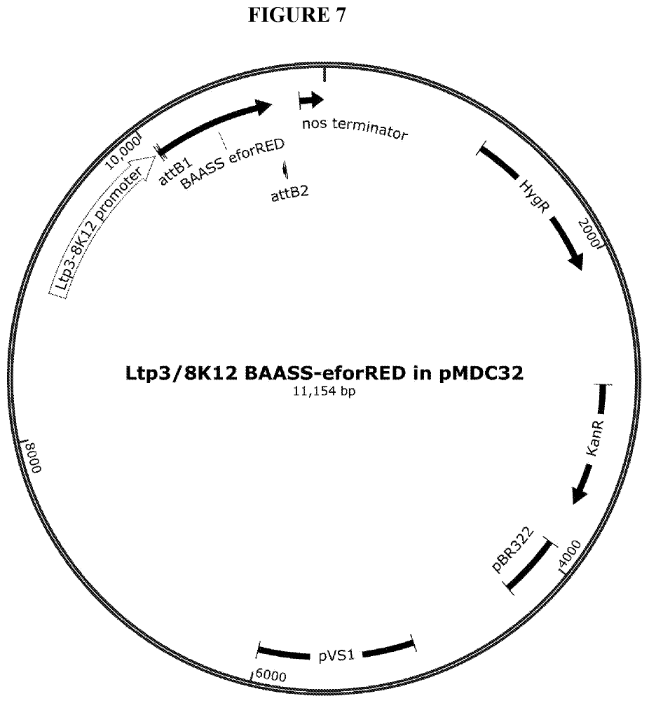

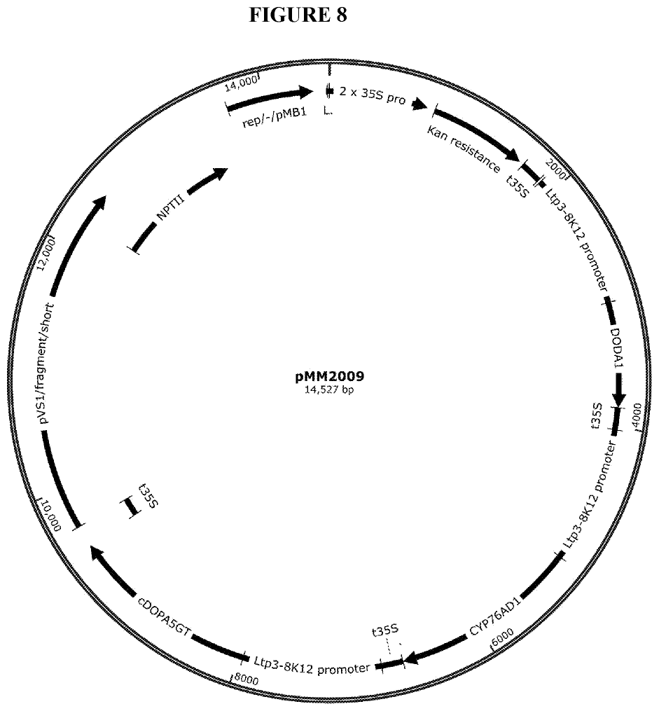

Constructs for the fibre-specific expression of one or more pigment producing genes are shown in FIGS. 6 to 8.

### Example 4—Fibre Quality

Naturally occurring colored cotton is characterized by reduced lint length and fineness. Such quality attributes are controlled by multiple genes that are susceptible to environmental factors, which negatively impact fibre development (Murthy, 2001, Resonance, December, 29-35). To assess the quality attributes of the colored cotton lines described herein, fibre samples obtained from the transgenic lines were assessed using Cottonscope, which measures cotton fibre fineness and maturity quickly and accurately in the same measurement.

Cotton bolls were obtained from transgenic cotton plants stably expressing the 2009 vector and wild type cotton plants (i.e., Coker 315) at different developmental stages. All cotton bolls were harvested on the specified days post-anthesis (DPA), or within two days of the specified DPA. Fresh and freeze-dried cotton bolls were used for the analysis. For each line tested, the number of samples (i.e., bolls) ranged from 3-6 obtained from the same transgenic line, or from different wild type plants.

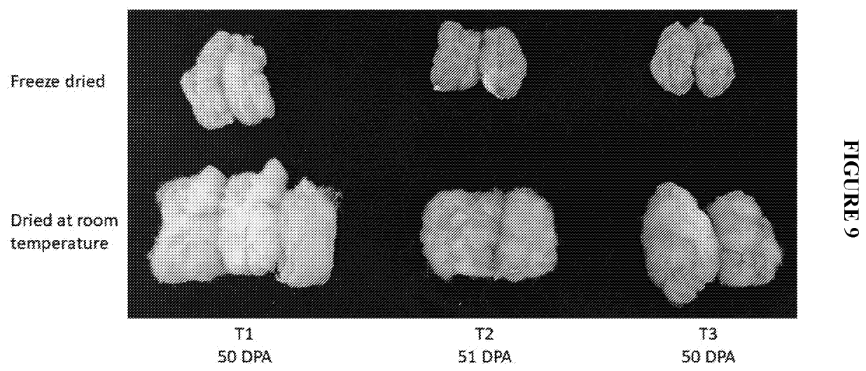

No significant difference in the fineness of the mature and immature fibres of the transgenic plants was observed, nor was there a significant difference between the transgenic lines and the wild type (FIG. 9A). However, the transgenic lines produced finer fibres as compared to the wild type cotton plant. The transgenic lines also produced fibre with a similar maturity ratio to the wild type cotton plant (FIG. 9B), which indicates that the heterologous expression of the pigment producing genes did not result in a significant reduction in fibre quality. These data demonstrate that the transgenic lines can produce colored fibre with the same, or improved fineness and similar maturity ratio as the wild type, which may be further refined by, e.g., by introgressing the Coker 315 cultivate into modern elite cotton varieties.

### Example 5—Production of Hybrid Cotton Seed

Cotton plants can be crossed with cotton plants that are transgenic for the nucleic acid construct described herein. Crossing can be natural or mechanical. The flowers of cotton plants comprise both the male and female parts in the same flower, as such cotton plants usually self-pollinate. To achieve the crossing of two selected cotton plants it is typical to use artificial hybridization for example the flower used as a female in a cross is manually cross-pollinated prior to maturation of pollen from the flower or the male parts of the flower can be emasculated using methods known in the art. Selection of plants to be used in a cross will be informed include a step to identify cotton plants having other desirable features, such as higher fibre (lint) yield, improved fibre characteristics, earlier maturity, disease resistance genes or reduced gossypol content of the seed. The F1 progeny plants can be backcrossed to a cotton plant as a recurrent parent for several generations to provide cotton plants with colored fibre.

The progeny with colored construct can be determined by molecular marker analysis or by analysis of the lint as described herein. Typically backcrossing procedures include crossing the original line of interest (recurrent parent) to a second variety (nonrecurrent parent) that carries the colored cotton genetic locus to be transferred. The backcrossing process may be assisted by the use of molecular markers.

### Example 6—Color Maintenance

Three independent transgenic lines were grown for T1 plants. Cotton bolls were collected between 45-53 DPA, or 45-50 DPA. To investigate whether the presence of water leads to pigment degradation, immature cotton bolls were dried using convective drying, contact drying, freeze drying or drying at room temperature.

In the first instance, immature cotton bolls with seeds were either: (i) freeze dried for 48 hours with the FTS Systems Flexi-Dry MP freeze dryer (−40° C., vacuum below 500 mT); or (ii) left at room temperature overnight (23-25° C.). As shown in FIG. 9, immature cotton fibres (with seeds) that were freeze dried or dried at room temperature retained a vivid pink color after maturation at around 50 DPA.

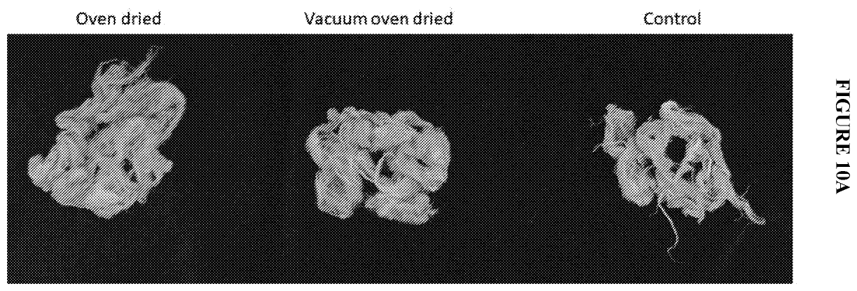

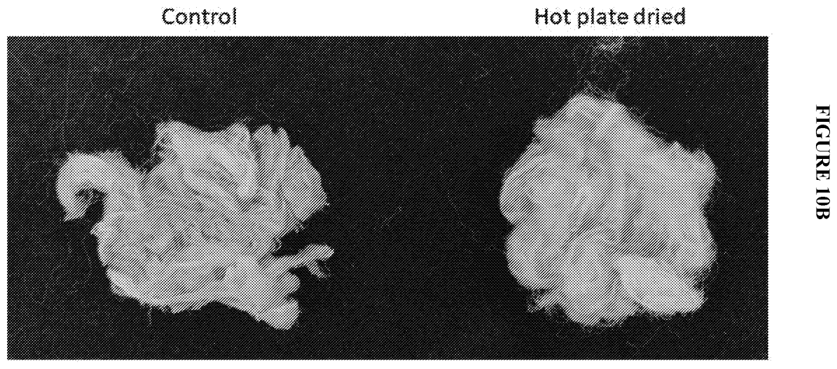

Thereafter, fresh ginned wet cotton fibres were either: (i) oven dried at 60° C. for 30 minutes with a vacuum pressure of −50 kPa; (ii) oven (i.e., convective) dried (60° C. for 30 minutes); (iii) left at room temperature overnight (23-25° C.); or (iv) dried for 5 minutes on a 200° C. hot plate (i.e., contact drying), turning sides frequently. As shown in FIG. 10, each of the drying techniques was sufficient to retain a vivid pink color, which was consistent with the color of the fresh ginned wet fibres.

Ethanol was also shown to be an effective fixative for cotton fibre.

Taken together, these data demonstrate that the presence of water leads to color degradation in transgenic cotton lines modified to express betalain genes. Therefore, removal of water by rapid drying methods can be used to maintain exogenous pigments in cotton fibre, even after maturation.

### Discussion

Naturally occurring colored cotton has been known for more than 5000 years in the Andes of South America ranging from deep chocolate, red, green, blue, mauve and many shades of brown (e.g., light brown, golden-brown, etc.) (Murthy, 2001, supra). However, these colored varieties tend to have poor fibre quality (e.g., short, coarse and/or weak), variable and unstable colors and low yield. In addition, modern cotton ginning and spinning processes require longer and stronger fibres, favoring the use of superior white cotton which is amenable to post-harvest dyeing (Vreeland, 1999, Scientific American, 280(4): 112-118). Consequently, colored cotton has historically been grown only in limited quantities and locations due to poor fibre quality and the risk of contamination of white fibres, which may result in price penalties due to decreased color grade (McVeigh, 2015, The impact of colour discounts to the Australian cotton industry, Report for Nuffield Project No. 1517).

Efforts to improve natural colored cotton have focused on conventional breeding techniques to address some of the issues associated with the poor fibre quality, such as producing varieties that are machine spinnable. However, while conventional breeding efforts have improved some of the fibre qualities of colored cotton, the fibre quality remains poor and the yield low compared to white cotton. Furthermore, the range of colors available is limited.

The present inventors have shown have shown that the exogenous pigments, such as chromoproteins or betalains, can be produced in the fibre cells of cotton plants by the expression of a nucleic acid construct comprising a nucleotide sequence comprising one or more pigment producing genes, wherein expression of the nucleotide sequence is under the control of a fibre-specific promoter, wherein the fibre-specific promoter is capable of driving expression of the nucleotide sequence during two or more fibre developmental stages. Using a fibre-specific promoter that drives expression across the mid-late stages of fibre development, betalain pigments were successfully produced in cotton fibres and resulted in visibly pink fibres, which were maintained in the inner boll (fibre and seeds) even after lypholization of bolls harvested at up to 59 DPA. Plants were either green or only partially purple (due to leaky expression in old tissues) and flowers resembled those of control plants at 0 DPA. By contrast, the 2×35S constitutive promoter driven construct generated purple/violet plants with purple leaves, stems and cotton boll coats and bracts. The flowers were pink upon opening (i.e., 0 DPA) while wild type cotton was green with creamy yellow flowers at 0 DPA. However, 2×35S promoter driven constructs only produced white fibres throughout the development, similar to those observed in wild type lines, despite the ovules being pink in the early developmental stages, as compared to the white ovules of the wild type plants.

The unexpected technical advantage of the approach described herein is the provision of colored cotton fibre that is produced in planta. Unlike previously reported attempts to generate novel colored cotton fibres using synthetic biology, e.g., blue indigo by Radik and Llewellyn (2000, Cotton Research Development Corporation (CRDC) Final Reports), black melanin by Xu et al. (2007, Plant Biology, 9(1): 41-48), and anthocyanins by Gong et al. (2018, in Cotton Fiber: Physics, Chemistry and Biology, 117-132), Yan et al. (2018, Plant Biotechnology Journal, 16(10): 1735-1747) and Wang et al. (2022, Plant Physiology, 00: 1-16), the inventors have produced a vivid pink cotton fibre, which remains colored through to the very late stages of fibre development.

Further, the selective expression of the one or more pigment producing genes in fibre cells has been shown to produce cotton plants with colored fibre without causing adverse effects to plant development or fibre quality, relative to wild type cotton plants. This outcome is unexpected, given that other known naturally occurring colored cotton plants suffer from severe fibre quality defects that make such varieties inappropriate for commercial fibre cultivation.

Taken together, these data enable the use of nucleic acid constructs comprising a nucleotide sequence comprising one or more pigment producing genes, wherein expression of the nucleotide sequence is under the control of a fibre-specific promoter, and wherein the fibre-specific promoter is capable of driving expression of the nucleotide sequence during two or more fibre developmental stages, to produce colored plant fibres in planta. The nucleic acid constructs, vectors, plants and associated products exemplified herein provide an example of colored dye-free cotton, which represents a beneficial alternative to the use of toxic and unsustainable synthetic dyes.

### Annotated Exemplary Sequences

SEQ ID NO: 31
Fibre-specific promoter ltp3/8K12 shown in bold; DODA1, CYP76D1 and cDOPA5GT,
shown respectively (from top to bottom) in italics; and 35S terminator shown in underline.
AAAATCACTTATCAATTTCAAAAACAGAGGTTAGCCGAATGCTAAGAGCTT
AAAAATGGCTTCTTTTGTTTCTTTTTCTTGCAAACGGTGGAGAGAAGAGG
GAAATGAAGATTGACCATATCTTTTTTTATTATGTTTTAACATATAATATTA
ATAATTTAATCATAATTATACTTTGGTGAATGTGACAGTGGGGATATACGT
AAAGTATATAACATTATACTTTTTGCAAGCAGTTGGCTGGTCTACCCAAGA
GTGATCAAATTTTGAGCTGCCTTCAATGAGCCAATTTTTGCCTATAATGGA
TAAAGGCACTTTGTCTAGTTCAACTGCTCACAGAATAATGTTAAAATGAAA
TTAAAACAAGGTGGGCTGGTCACACCAAAAAAATAAAATATTAATGTGGT
GTTTGGTTAGTCGATTTTATATTAGTTCCATGGCATACCGCCTGGAAAAGG
AAAATTCATGTAAATAATATATTTATAAAAATTTATATTAAAACTAAATGA
AATTTTAGTTGAAATAGTTAAGTTAAAAAGAGTAAAATTTATAATTTATCA
TAATTTTATAGAAATTGAGACTAAAAAACATTAAGAGAATAAATTCTATAA
CAAAGACAATTTAGTAAAAATGTCCTTTTAGGTAATTTTAAGTACTCTTAA
CCAAAATAAAAAATTCAAATCAAATTAACCAAATAAGATAATATAACATAC
GGAACATCCCACTTATAATCTTACATCCCCGTAATCTTATTATGAAAAGTA
ATCTTATATTACTCGAATCAAATGCTCTCACAAACTATTATCTAAATAAAG
AAAAACACTTAATTTTTATAACATTTTTTTTCATATATTTGAAAGATTATAT
TTTGTATTTTTACGTAAAAATATTTGACATAGATTGAGCACCTTTTTAACA
TAATTCCACCATAAGTCAATTATGTAGATGAGAAATTGGTACAAACAACGT
GGAGCCAAATCCCACCAAACCATCTCTCATCCTCTCCTATAAAAGGCTTGC
TACACATAGACAACAATCCACACAAAAATACACTTAAAATTCTTTTCTTTC
TATTTGGTTAACCGGTACCAATGAAGATGATGAACGGCGAGGACGCCAACGACC
AGATGATCAAAGAGAGCTTCTTCATCACCCACGGGAACCCTATCCTCACTGTTGAGG
ATACTCATCCTCTCAGGCCGTTCTTCGAAACTTGGAGGGAAAAGATCTTCAGCAAGA
AGCCGAAGGCCATCCTCATCATCTCTGGACATTGGGAGACTGTGAAGCCTACTGTG
AACGCTGTGCACATCAACGATACCATCCACGACTTCGATGACTACCCGGCTGCTATG
TACCAGTTCAAGTATCCTGCTCCTGGTGAGCCTGAGCTTGCTAGAAAGGTTGAAGAG
ATCCTCAAGAAGTCCGGGTTCGAGACTGCTGAGACTGATCAAAAGAGGGGTCTTGA
TCACGGTGCTTGGGTTCCACTTATGCTCATGTATCCTGAGGCTGACATCCCTGTGTG
CCAGCTTTCTGTTCAGCCTCATCTCGATGGAACCTACCACTACAATCTTGGAAGGGC
TCTTGCTCCGCTCAAGAACGATGGTGTTCTCATCATCGGAAGCGGGTCTGCTACACA
TCCTCTTGATGAGACTCCTCACTACTTCGATGGTGTGGCTCCTTGGGCTGCTGCTTT
TGATTCTTGGCTTCGTAAGGCCCTCATCAACGGTAGATTCGAAGAGGTGAACATCTA
CGAGAGCAAGGCTCCTAACTGGAAGCTCGCTCATCCATTTCCAGAGCACTTCTACCC
TCTCCATGTTGTTCTTGGAGCTGCTGGTGAAAAGTGGAAGGCTGAGCTTATCCACTC
TTCTTGGGATCATGGAACCCTCTGCCACGGGTCTTACAAGTTCACTTCTGCTTGA
GCTTCTCTAGCTAGAGTCGATCGACAAGCTCGAGTTTCTCCATAATAATGTGTG
AGTAGTTCCCAGATAAGGGAATTAGGGTTCCTATAGGGTTTCGCTCATGTGTTG
AGCATATAAGAAACCCTTAGTATGTATTTGTATTTGTAAAATACTTCTATCAAT
AAAATTTCTAATTCCTAAAACCAAAATCCAGTACTAAAATCCAGATCGCTACT
AGAATTCGAGCTCGGAGAAGCTTAAAATCACTTATCAATTTCAAAAACAGAG
GTTAGCCGAATGCTAAGAGCTTAAAAATGGCTTCTTTTGTTTCTTTTTCTT
GCAAACGGTGGAGAGAAGAGGGAAATGAAGATTGACCATATCTTTTTTTA
TTATGTTTTAACATATAATATTAATAATTTAATCATAATTATACTTTGGTGA
ATGTGACAGTGGGGATATACGTAAAGTATATAACATTATACTTTTTGCAAG
CAGTTGGCTGGTCTACCCAAGAGTGATCAAATTTTGAGCTGCCTTCAATG
AGCCAATTTTTGCCTATAATGGATAAAGGCACTTTGTCTAGTTCAACTGCT
CACAGAATAATGTTAAAATGAAATTAAAACAAGGTGGGCTGGTCACACCA
AAAAAATAAAATATTAATGTGGTGTTTGGTTAGTCGATTTTATATTAGTTC
CATGGCATACCGCCTGGAAAAGGAAAATTCATGTAAATAATATATTTATAA
AAATTTATATTAAAACTAAATGAAATTTTAGTTGAAATAGTTAAGTTAAAA
AGAGTAAAATTTATAATTTATCATAATTTTATAGAAATTGAGACTAAAAAA
CATTAAGAGAATAAATTCTATAACAAAGACAATTTAGTAAAAATGTCCTTT
TAGGTAATTTTAAGTACTCTTAACCAAAATAAAAAATTCAAATCAAATTAA
CCAAATAAGATAATATAACATACGGAACATCCCACTTATAATCTTACATCC
CCGTAATCTTATTATGAAAAGTAATCTTATATTACTCGAATCAAATGCTCT
CACAAACTATTATCTAAATAAAGAAAAACACTTAATTTTTATAACATTTTTT
TTCATATATTTGAAAGATTATATTTTGTATTTTTACGTAAAAATATTTGACA
TAGATTGAGCACCTTTTTAACATAATTCCACCATAAGTCAATTATGTAGAT
GAGAAATTGGTACAAACAACGTGGAGCCAAATCCCACCAAACCATCTCTC
ATCCTCTCCTATAAAAGGCTTGCTACACATAGACAACAATCCACACAAAAA
TACACTTAAAATTCTTTTCTTTCTATTTGGTTAACCGGTACCAATGGATCATG
CTACCCTCGCTATGATCCTCGCGATCTGGTTCATCAGCTTCCACTTCATCAAGCTCTT
GTTCAGCCAGCAGACGACCAAGCTTCTTCCTCCTGGACCTAAACCTCTTCCGATCAT
CGGTAACATCCTCGAGGTTGGAAAGAAGCCGCACCGTTCTTTTGCTAACCTGGCTAA
GATCCACGGACCGCTCATTTCTCTTAGACTCGGATCTGTGACGACCATCGTGGTGTC
ATCTGCTGATGTGGCCAAAGAGATGTTCCTCAAGAAGGATCACCCGCTCAGCAACA
GGACTATCCCTAATTCTGTGACCGCTGGTGACCACCACAAGCTGACTATGTCTTGGC
TTCCTGTGTCTCCGAAGTGGCGAAACTTCCGTAAGATTACTGCTGTGCACCTCTTGT
CTCCACAGAGACTTGATGCTTGCCAGACCTTCAGACATGCTAAGGTTCAGCAGCTCT
ACGAGTACGTTCAAGAGTGTGCTCAGAAAGGACAGGCTGTGGATATTGGCAAGGCT
GCCTTTACTACCAGCCTCAACCTTCTGTCCAAGCTGTTCTTCAGCGTTGAGCTTGCT
CACCACAAGAGCCACACTAGCCAAGAGTTCAAAGAGCTGATCTGGAACATCATGGAA
GATATCGGGAAGCCGAACTACGCTGACTACTTCCCTATCCTCGGATGCGTTGACCCT
TCTGGAATCAGAAGAAGGCTCGCTTGCTCTTTCGACAAGCTGATCGCTGTTTTCCAG
GGGATCATCTGCGAAAGACTCGCTCCTGATTCTTCTACCACTACCACCACTACTACC
GACGACGTTCTCGATGTGCTTCTCCAGCTTTTCAAGCAGAACGAGCTTACCATGGGC
GAGATCAACCATCTCCTCGTGGATATCTTCGATGCTGGAACCGATACCACCAGCTCT
ACTTTCGAGTGGGTGATGACCGAGCTTATCAGAAACCCTGAGATGATGGAAAAGGC
CCAAGAAGAGATCAAGCAGGTCCTCGGAAAGGACAAGCAGATCCAAGAGAGCGACA
TCATCAACCTTCCATACCTCCAGGCCATCATCAAAGAGACTCTCAGACTCCATCCTC
CGACCGTTTTCTTGCTTCCTAGAAAGGCTGATACCGACGTCGAGCTGTACGGTTACA
TCGTTCCTAAGGATGCTCAGATCCTCGTGAACCTTTGGGCTATCGGTAGAGATCCTA
ACGCTTGGCAGAACGCCGACATTTTTAGCCCTGAGAGATTCATCGGATGCGAGATC
GATGTGAAGGGTCGTGATTTCGGACTTCTCCCATTTGGAGCTGGTAGAAGGATCTGC
CCTGGAATGAACCTCGCTATCAGAATGCTCACCCTTATGCTCGCTACTCTCCTCCAG
TTCTTCAACTGGAAGCTCGAGGGTGACATCTCTCCTAAGGACCTCGATATGGACGAG
AAGTTCGGAATCGCTCTCCAAAAGACCAAGCCGCTTAAGCTCATCCCTATTCCGAGG
TACTGAGCTTCTCTAGCTAGAGTCGATCGACAAGCTCGAGTTTCTCCATAATAA
TGTGTGAGTAGTTCCCAGATAAGGGAATTAGGGTTCCTATAGGGTTTCGCTCAT
GTGTTGAGCATATAAGAAACCCTTAGTATGTATTTGTATTTGTAAAATACTTCT
ATCAATAAAATTTCTAATTCCTAAAACCAAAATCCAGTACTAAAATCCAGATC
GCTTTACGAATTCCCATGGGGAGAAGCTTAAAATCACTTATCAATTTCAAAA
ACAGAGGTTAGCCGAATGCTAAGAGCTTAAAAATGGCTTCTTTTGTTTCTT
TTTCTTGCAAACGGTGGAGAGAAGAGGGAAATGAAGATTGACCATATCTT
TTTTTATTATGTTTTAACATATAATATTAATAATTTAATCATAATTATACTT
TGGTGAATGTGACAGTGGGGATATACGTAAAGTATATAACATTATACTTTT
TGCAAGCAGTTGGCTGGTCTACCCAAGAGTGATCAAATTTTGAGCTGCCT
TCAATGAGCCAATTTTTGCCTATAATGGATAAAGGCACTTTGTCTAGTTCA
ACTGCTCACAGAATAATGTTAAAATGAAATTAAAACAAGGTGGGCTGGTC
ACACCAAAAAAATAAAATATTAATGTGGTGTTTGGTTAGTCGATTTTATAT
TAGTTCCATGGCATACCGCCTGGAAAAGGAAAATTCATGTAAATAATATAT
TTATAAAAATTTATATTAAAACTAAATGAAATTTTAGTTGAAATAGTTAAG
TTAAAAAGAGTAAAATTTATAATTTATCATAATTTTATAGAAATTGAGACT
AAAAAACATTAAGAGAATAAATTCTATAACAAAGACAATTTAGTAAAAATG
TCCTTTTAGGTAATTTTAAGTACTCTTAACCAAAATAAAAAATTCAAATCA
AATTAACCAAATAAGATAATATAACATACGGAACATCCCACTTATAATCTT
ACATCCCCGTAATCTTATTATGAAAAGTAATCTTATATTACTCGAATCAAA
TGCTCTCACAAACTATTATCTAAATAAAGAAAAACACTTAATTTTTATAAC
ATTTTTTTTCATATATTTGAAAGATTATATTTTGTATTTTTACGTAAAAATA
TTTGACATAGATTGAGCACCTTTTTAACATAATTCCACCATAAGTCAATTA
TGTAGATGAGAAATTGGTACAAACAACGTGGAGCCAAATCCCACCAAACC
ATCTCTCATCCTCTCCTATAAAAGGCTTGCTACACATAGACAACAATCCAC
ACAAAAATACACTTAAAATTCTTTTCTTTCTATTTGGTTAACCGGTACCAAT
GACCGCCATCAAGATGAACACCAACGGTGAGGGTGAGACTCAGCACATCCTTATGA
TCCCTTTCATGGCTCAGGGACACCTCAGACCATTICTCGAGCTTGCTATGTTCCTCTA
CAAGCGTAGCCACGTGATCATCACACTCCTCACTACTCCTCTCAACGCTGGATTCCT
TAGACATCTCCTCCACCACCACAGCTACAGCTCATCTGGAATCAGGATCGTCGAGCT
GCCTTTCAACTCTACTAACCATGGACTCCCACCGGGAATCGAGAATACTGATAAGCT
TACTCTCCCGCTCGTGGTGAGCCTCTTCCATTCTACTATTTCTCTCGATCCGCACCTC
AGGGACTACATCTCTAGACATTTCAGCCCTGCTAGGCCTCCTCTCTGCGTTATCCAT
GATGTTTTTCTCGGCTGGGTTGACCAGGTGGCAAAGGATGTTGGATCTACTGGTGTG
GTGTTCACTACCGGTGGTGCTTATGGAACCTCTGCCTACGTTTCGATCTGGAACGAT
CTCCCTCACCAGAACTACTCTGACGACCAAGAATTCCCTCTTCCGGGATTCCCTGAG
AACCACAAGTTCAGAAGATCTCAGCTCCACCGTTTCCTCAGGTACGCTGATGGATCT
GATGACTGGTCCAAGTACTTCCAGCCTCAGCTCAGACAGAGCATGAAGTCTTTCGGA
TGGCTCTGCAACAGCGTGGAAGAGATTGAGACTCTCGGGTTCTCTATCCTCCGTAAC
TACACTAAGCTCCCGATCTGGGGAATCGGACCTCTTATTGCTTCTCCTGTGCAGCAC
TCCAGCAGCGATAACAATTCTACAGGTGCTGAGTTCGTGCAGTGGCTCTCTTTGAAA
GAGCCTGACTCTGTCCTCTACATCAGCTTCGGAAGCCAGAACACCATCTCTCCGACT
CAGATGATGGAACTTGCTGCTGGACTTGAGTCCTCTGAGAAGCCATTCCTTTGGGTT
ATCAGGGCTCCTTTCGGGTTCGACATCAACGAAGAAATGAGGCCTGAGTGGCTTCC
TGAGGGATTCGAAGAGAGGATGAAGGTCAAGAAGCAGGGGAAGCTCGTTTACAAGC
TTGGACCTCAGCTTGAGATCCTCAACCACGAGTCTATCGGAGGATTCCTCACTCATT
GCGGCTGGAACTCTATCCTTGAGTCTCTTAGAGAAGGGGTGCCAATGCTTGGATGG
CCTTTGGCTGCTGAACAGGCTTACAACCTTAAGTACCTCGAGGACGAGATGGGAGTT
GCTGTTGAACTTGCTAGAGGACTCGAAGGGGAGATCAGCAAAGAGAAGGTTAAGCG
TATCGTCGAGATGATCCTCGAGAGGAACGAGGGATCTAAAGGCTGGGAGATGAAGA
ACAGGGCTGTTGAGATGGGGAAGAAGCTCAAGGACGCTGTCAACGAGGAAAAAGA
GCTGAAGGGGTCAAGCGTGAAGGCTATCGATGATTTCCTCGACGCTGTGATGCAGG
CTAAGCTTGAGCCATCTCTTCAGTGAGCTTCTCTAGCTAGAGTCGATCGACAAGC
TCGAGTTTCTCCATAATAATGTGTGAGTAGTTCCCAGATAAGGGAATTAGGGT
TCCTATAGGGTTTCGCTCATGTGTTGAGCATATAAGAAACCCTTAGTATGTATT
TGTATTTGTAAAATACTTCTATCAATAAAATTTCTAATTCCTAAAACCAAAATC
CAGTACTAAAATCCAGAT
SEQ ID NO: 32
CYP76D1, DODA1 and cDOPA5GT, shown respectively (from top to bottom) in italics; and
2A linker sequence shown in bold.
ATGGATCATGCTACCCTCGCTATGATCCTCGCGATCTGGTTCATCAGCTTCCACTTC
ATCAAGCTCTTGTTCAGCCAGCAGACGACCAAGCTTCTTCCTCCTGGACCTAAACCT
CTTCCGATCATCGGTAACATCCTCGAGGTTGGAAAGAAGCCGCACCGTTCTTTTGCT
AACCTGGCTAAGATCCACGGACCGCTCATTTCTCTTAGACTCGGATCTGTGACGACC
ATCGTGGTGTCATCTGCTGATGTGGCCAAAGAGATGTTCCTCAAGAAGGATCACCCG
CTCAGCAACAGGACTATCCCTAATTCTGTGACCGCTGGTGACCACCACAAGCTGACT
ATGTCTTGGCTTCCTGTGTCTCCGAAGTGGCGAAACTTCCGTAAGATTACTGCTGTG
CACCTCTTGTCTCCACAGAGACTTGATGCTTGCCAGACCTTCAGACATGCTAAGGTT
CAGCAGCTCTACGAGTACGTTCAAGAGTGTGCTCAGAAAGGACAGGCTGTGGATAT
TGGCAAGGCTGCCTTTACTACCAGCCTCAACCTTCTGTCCAAGCTGTTCTTCAGCGT
TGAGCTTGCTCACCACAAGAGCCACACTAGCCAAGAGTTCAAAGAGCTGATCTGGAA
CATCATGGAAGATATCGGGAAGCCGAACTACGCTGACTACTTCCCTATCCTCGGATG
CGTTGACCCTTCTGGAATCAGAAGAAGGCTCGCTTGCTCTTTCGACAAGCTGATCGC
TGTTTTCCAGGGGATCATCTGCGAAAGACTCGCTCCTGATTCTTCTACCACTACCAC
CACTACTACCGACGACGTTCTCGATGTGCTTCTCCAGCTTTTCAAGCAGAACGAGCT
TACCATGGGCGAGATCAACCATCTCCTCGTGGATATCTTCGATGCTGGAACCGATAC
CACCAGCTCTACTTTCGAGTGGGTGATGACCGAGCTTATCAGAAACCCTGAGATGAT
GGAAAAGGCCCAAGAAGAGATCAAGCAGGTCCTCGGAAAGGACAAGCAGATCCAAG
AGAGCGACATCATCAACCTTCCATACCTCCAGGCCATCATCAAAGAGACTCTCAGAC
TCCATCCTCCGACCGTTTTCTTGCTTCCTAGAAAGGCTGATACCGACGTCGAGCTGT
ACGGTTACATCGTTCCTAAGGATGCTCAGATCCTCGTGAACCTTTGGGCTATCGGTA
GAGATCCTAACGCTTGGCAGAACGCCGACATTTTTAGCCCTGAGAGATTCATCGGAT
GCGAGATCGATGTGAAGGGTCGTGATTTCGGACTTCTCCCATTTGGAGCTGGTAGA
AGGATCTGCCCTGGAATGAACCTCGCTATCAGAATGCTCACCCTTATGCTCGCTACT
CTCCTCCAGTTCTTCAACTGGAAGCTCGAGGGTGACATCTCTCCTAAGGACCTCGAT
ATGGACGAGAAGTTCGGAATCGCTCTCCAAAAGACCAAGCCGCTTAAGCTCATCCCT
ATTCCGAGGTACTGAGGAAGCGGAGCTACTAACTTCAGCCTGCTGAAGCAG
GCTGGAGACGTGGAGGAGAACCCTGGACCTATGAAGATGATGAACGGCGAGG
ACGCCAACGACCAGATGATCAAAGAGAGCTTCTTCATCACCCACGGGAACCCTATCC
TCACTGTTGAGGATACTCATCCTCTCAGGCCGTTCTTCGAAACTTGGAGGGAAAAGA
TCTTCAGCAAGAAGCCGAAGGCCATCCTCATCATCTCTGGACATTGGGAGACTGTGA
AGCCTACTGTGAACGCTGTGCACATCAACGATACCATCCACGACTTCGATGACTACC
CGGCTGCTATGTACCAGTTCAAGTATCCTGCTCCTGGTGAGCCTGAGCTTGCTAGAA
AGGTTGAAGAGATCCTCAAGAAGTCCGGGTTCGAGACTGCTGAGACTGATCAAAAG
AGGGGTCTTGATCACGGTGCTTGGGTTCCACTTATGCTCATGTATCCTGAGGCTGAC
ATCCCTGTGTGCCAGCTTTCTGTTCAGCCTCATCTCGATGGAACCTACCACTACAAT
CTTGGAAGGGCTCTTGCTCCGCTCAAGAACGATGGTGTTCTCATCATCGGAAGCGG
GTCTGCTACACATCCTCTTGATGAGACTCCTCACTACTTCGATGGTGTGGCTCCTTG
GGCTGCTGCTTTTGATTCTTGGCTTCGTAAGGCCCTCATCAACGGTAGATTCGAAGA
GGTGAACATCTACGAGAGCAAGGCTCCTAACTGGAAGCTCGCTCATCCATTTCCAGA
GCACTTCTACCCTCTCCATGTTGTTCTTGGAGCTGCTGGTGAAAAGTGGAAGGCTGA
GCTTATCCACTCTTCTTGGGATCATGGAACCCTCTGCCACGGGTCTTACAAGTTCAC
TTCTGCTTGAGGTTCAGGTGCCACTAATTTTTCTCTCTTGAAACAGGCCGGT
GACGTTGAAGAGAACCCAGGTCCAATGACCGCCATCAAGATGAACACCAACGG
TGAGGGTGAGACTCAGCACATCCTTATGATCCCTTTCATGGCTCAGGGACACCTCAG
ACCATTICTCGAGCTTGCTATGTTCCTCTACAAGCGTAGCCACGTGATCATCACACTC
CTCACTACTCCTCTCAACGCTGGATTCCTTAGACATCTCCTCCACCACCACAGCTAC
AGCTCATCTGGAATCAGGATCGTCGAGCTGCCTTTCAACTCTACTAACCATGGACTC
CCACCGGGAATCGAGAATACTGATAAGCTTACTCTCCCGCTCGTGGTGAGCCTCTTC
CATTCTACTATTTCTCTCGATCCGCACCTCAGGGACTACATCTCTAGACATTTCAGCC
CTGCTAGGCCTCCTCTCTGCGTTATCCATGATGTTTTTCTCGGCTGGGTTGACCAGG
TGGCAAAGGATGTTGGATCTACTGGTGTGGTGTTCACTACCGGTGGTGCTTATGGAA
CCTCTGCCTACGTTTCGATCTGGAACGATCTCCCTCACCAGAACTACTCTGACGACC
AAGAATTCCCTCTTCCGGGATTCCCTGAGAACCACAAGTTCAGAAGATCTCAGCTCC
ACCGTTTCCTCAGGTACGCTGATGGATCTGATGACTGGTCCAAGTACTTCCAGCCTC
AGCTCAGACAGAGCATGAAGTCTTTCGGATGGCTCTGCAACAGCGTGGAAGAGATT
GAGACTCTCGGGTTCTCTATCCTCCGTAACTACACTAAGCTCCCGATCTGGGGAATC
GGACCTCTTATTGCTTCTCCTGTGCAGCACTCCAGCAGCGATAACAATTCTACAGGT
GCTGAGTTCGTGCAGTGGCTCTCTTTGAAAGAGCCTGACTCTGTCCTCTACATCAGC
TTCGGAAGCCAGAACACCATCTCTCCGACTCAGATGATGGAACTTGCTGCTGGACTT
GAGTCCTCTGAGAAGCCATTCCTTTGGGTTATCAGGGCTCCTTTCGGGTTCGACATC
AACGAAGAAATGAGGCCTGAGTGGCTTCCTGAGGGATTCGAAGAGAGGATGAAGGT
CAAGAAGCAGGGGAAGCTCGTTTACAAGCTTGGACCTCAGCTTGAGATCCTCAACC
ACGAGTCTATCGGAGGATTCCTCACTCATTGCGGCTGGAACTCTATCCTTGAGTCTC
TTAGAGAAGGGGTGCCAATGCTTGGATGGCCTTTGGCTGCTGAACAGGCTTACAAC
CTTAAGTACCTCGAGGACGAGATGGGAGTTGCTGTTGAACTTGCTAGAGGACTCGA
AGGGGAGATCAGCAAAGAGAAGGTTAAGCGTATCGTCGAGATGATCCTCGAGAGGA
ACGAGGGATCTAAAGGCTGGGAGATGAAGAACAGGGCTGTTGAGATGGGGAAGAA
GCTCAAGGACGCTGTCAACGAGGAAAAAGAGCTGAAGGGGTCAAGCGTGAAGGCT
ATCGATGATTTCCTCGACGCTGTGATGCAGGCTAAGCTTGAGCCATCTCTTCAGTGA

TABLE 1
Transgenic cotton lines comprising nucleic acid constructs encoding
betalain pathway genes

| Trans- | | | | | |
|---|---|---|---|---|---|
| formation | Vector | Promoter | Gene 1 | Gene 2 | Gene 3 |
| 544 (purple; | pMM2001 | 2 × 35S | DODA1 | CYP76AD1 | cDOPA5GT |
| whole plant) | | | | | |
| 542 (yellow/ | pMM2003 | 2 × 35S | DODA1 | CYP76AD6 | — |
| green; whole | | | | | |
| plant) | | | | | |
| 553 (purple; | pMM2009 | ltp3/ | DODA1 | CYP76AD1 | cDOPA5GT |
| fibre only) | | 8K12 | | | |

TABLE 2
Spectroscopic characteristics of chromoproteins (reproduced from
Liljeruhm et al., 2018, Journal of Biomedical Engineering, 12:8)

| Chromoprotein/pigment | Max. excitation (nm) | Max. emission (nm) |
|---|---|---|
| meffRed | 560 | 576 |
| eforRED | 589 | 609 |
| asPink | 568 | 595 |
| spisPink | 560 | NF |
| amilGFP | 503 | 512 |
| amajLime | 458 | 486 |
| cjBlue | 610 | NF |
| meffBlue | 592 | NF |
| aeBlue | 597 | NF |
| amilCP | 588 | NF |
| gfasPurple | 577 | NF |

NF—non-fluorescent.

The claims defining the invention are as follows:

1. A nucleic acid construct comprising a nucleotide sequence comprising one or more pigment producing genes, wherein expression of the nucleotide sequence is under the control of a cotton fibre-specific promoter, and wherein the cotton fibre-specific promoter is capable of driving expression of the nucleotide sequence during two or more fibre development stages.

2. The nucleic acid construct of claim 1, wherein the cotton fibre-specific promoter is ltp3/8K12, or a promoter of a cotton fibre-specific gene selected from the group consisting of FLA7, FLA12, FLA9, FLA11 and FLA17.

3. The nucleic acid construct of claim 2, wherein the cotton fibre-specific promoter is ltp3/8K12.

4. The nucleic acid construct of any one of claims 1 to 3, wherein the one or more pigment producing genes are exogenous pigment producing genes, and wherein the exogenous pigment producing genes are non-native to cotton.

5. The nucleic acid construct of any one of claims 1 to 4, wherein the two or more fibre development stages includes secondary wall deposition, being from about 15 to about 45 days post-anthesis (DPA).

6. The nucleic acid construct of claim 5, wherein the two or more fibre development stages includes elongation, being from about 5 to about 25 DPA.

7. The nucleic acid construct of any one of claims 1 to 6, wherein the cotton fibre-specific promoter is capable of driving expression of the nucleotide sequence at a level that is sufficient to produce pigment that is present in cotton fibre at ≥40 DPA.

8. The nucleic acid construct of any one of claims 1 to 7, wherein the one or more pigment producing genes encodes a pigment or an enzyme required to produce a pigment.

9. The nucleic acid construct of claim 8, wherein the pigment is melanin.

10. The nucleic acid construct of claim 8, wherein the pigment is selected from the group consisting of a chromoprotein, a betalain, an anthocyanin, a carotenoid and an indigoidine.

11. The nucleic acid construct of claim 10, wherein the chromoprotein is selected from the group consisting of eforRED (Echinopora forskaliana), amilGFP (Acropora millepora), amilCP (Acropora millepora) and combinations of the foregoing.

12. The nucleic acid construct of claim 10, wherein the betalain is a betacyanin.

13. The nucleic acid construct of claim 12, wherein the betacyanin is betanin.

14. The nucleic acid construct of claim 10, wherein the betalain is a betaxanthin.

15. The nucleic acid construct of claim 14, wherein the betaxanthin is vulgaxanthin I.

16. The nucleic acid construct of any one of claims 1 to 15, wherein the one or more pigment producing genes is selected from the group consisting of cDOPA5GT, DODA1, CYP76AD1, CYP76AD6, amilGFP, amilCP, eforRED, igiA, igiD, igiB and combinations of the foregoing.

17. The nucleic acid construct of claim 16, wherein the one or more pigment producing genes is selected from the group consisting of cDOPA5GT, DODA1, CYP76AD1, CYP76AD6 and combinations of the foregoing.

18. The nucleic acid construct of claim 17, wherein the one or more pigment producing genes comprise DODA1, CYP76AD1 and cDOPA5GT.

19. The nucleic acid construct of claim 17, wherein the one or more pigment producing genes comprise DODA1 and CYP76AD6.

20. The nucleic acid construct of claim 16, wherein the one or more pigment producing genes is selected from the group consisting of amilGFP, amilCP, eforRED, and combinations of the foregoing.

21. The nucleic acid construct of any one of claims 1 to 20, further comprising a nucleotide sequence encoding one or both of (i) a signal peptide; and (ii) an anchoring domain.

22. The nucleic acid construct of claim 21, wherein the signal peptide is barley α-amylase signal sequence (BAASS) or a cotton fasciclin-like arabinogalatan (FLA) signal peptide.

23. The nucleic acid construct of claim 22, wherein the anchoring domain is a carbohydrate binding domain.

24. The nucleic acid construct of claim 23, wherein the carbohydrate binding domain is a cellulose binding domain.

25. The nucleic acid construct of claim 1, comprising a nucleotide sequence selected from the group consisting of SEQ ID NOs: 1, 3, 5, 7, 9, 11 and a sequence having 90% identity to any of the foregoing.

26. A nucleic acid construct comprising a nucleotide sequence comprising one or more pigment producing genes, wherein the one or more pigment producing genes are exogenous pigment producing genes that are non-native to cotton, wherein expression of the nucleotide sequence is under the control of a cotton fibre-specific promoter, wherein the cotton fibre-specific promoter is capable of driving expression of the nucleotide sequence during two or more fibre development stages, and wherein the cotton fibre-specific promoter is capable of driving expression of the nucleotide sequence at a level that is sufficient to produce pigment that is present in cotton fibre at ≥40 DPA.

27. A vector comprising the nucleic acid construct of any one of claims 1 to 26.

28. A cotton plant cell comprising the nucleic acid construct of any one of claims 1 to 26, or the vector of claim 27.

29. A polypeptide encoded by the nucleic acid construct of any one of claims 1 to 26.

30. A method of producing a colored cotton fibre, the method comprising: (i) transforming a cotton plant cell with the nucleic acid construct of any one of claims 1 to 26, or the vector of claim 27; (ii) regenerating a cotton plant from the transformed cell; and (iii) harvesting the fibre from the regenerated cotton plant.

31. The method of claim 30, further comprising the step of crossing the regenerated cotton plant in step (ii) with a second cotton plant to produce a progeny cotton plant.

32. The method of claim 31, wherein the second cotton plant is of a different genetic background to the regenerated cotton plant in step (ii).

33. A method of introducing one or more pigment producing genes into a cotton plant, wherein the one or more pigment producing genes are heterologous to the cotton plant, the method comprising:
(i) crossing a first parent cotton plant with a second parent cotton plant, wherein said second parent cotton plant is transgenic for the nucleic acid construct of any one of claims 1 to 26; and
(ii) backcrossing a progeny plant of the cross of step (i) with a cotton plant of the same genotype as the first parent cotton plant to produce a cotton plant with a majority of the genotype of the first parent cotton plant but characterized by the expression of the one or more pigment producing genes in the fibre cells of the cotton plant.

34. The method of claim 33, further comprising genotyping the progeny plant for the presence or absence of the one or more pigment producing genes using a method of genotyping a cotton plant, the method comprising: (i) obtaining a sample comprising nucleic acid or protein extracted from the progeny plant, and (ii) detecting in the sample the nucleic acid construct of any one of claims 1 to 26, or the polypeptide of claim 29.

35. The method of claim 33 or claim 34, further comprising analyzing the optical properties of the fibres of the progeny plant for the presence or absence of one or more exogenous pigments, the method comprising: (i) obtaining a sample comprising fibre cells from the progeny plant, and (ii) using a spectroscopic method to detect pigments across visible or non-visible wavelengths.

36. A cotton plant modified to produce colored fibre, wherein the cotton plant comprises the nucleic acid construct of any one of claims 1 to 26, and wherein the one or more pigment producing genes are expressed in fibre cells of the cotton plant.

37. A method of producing cotton seed, wherein the method comprises crossing the plant of claim 36 with itself or a second, distinct cotton plant to produce cotton seed, wherein the cotton seed comprises the nucleic acid construct of any one of claims 1 to 26.

38. The method of claim 37, further comprising: (i) growing a progeny plant from a subsequent generation from the cotton seed and crossing the progeny plant of a subsequent generation with itself or a second cotton plant; and (ii) repeating step (i) for additional generations with sufficient inbreeding to produce seed of an inbred cotton plant, wherein the seed of the inbred cotton plant comprises the nucleic acid construct of any one of claims 1 to 26.

39. Cotton seed produced by the method of claim 37 or claim 38.

40. A composition comprising the cotton seed of claim 39, wherein the seed is comprised in plant seed growth media, and wherein said plant seed growth media comprises soil or synthetic cultivation media.

41. A method of producing a commodity plant product, wherein the method comprises collecting the commodity plant product from the cotton plant of claim 36.

42. The method of claim 41, wherein the commodity plant product is cotton lint or fibre.

43. A method of producing cotton lint, the method comprising: (i) growing the cotton plant of claim 36; and (ii) harvesting the cotton lint.

44. The method of claim 43, further comprising drying the harvested cotton lint.

45. The method of claim 44, wherein the harvested cotton lint is dried using a method selected from the group consisting of convective drying, contact drying, dielectric drying and freeze drying.

46. The method of claim 44 or claim 45, wherein the dried harvested cotton lint has a dry basis moisture content of less than about 7% by weight.

47. The method of any one of claims 44 to 46, wherein the harvested cotton lint is dehydrated.

48. The method of claim 43, further comprising fixing the harvested cotton lint in a solvent comprising alcohol.

49. The method of claim 48, wherein the alcohol is selected from the group consisting of ethanol, propan-2-ol and trichloroethene.

50. Cotton lint produced by the method of any one of claims 43 to 49.

51. Cotton lint obtained from the cotton plant of claim 36.

52. The cotton lint of claim 50 or claim 51, comprising fibre with an average fineness of from about 160 mTex to about 240 mTex.

53. The cotton lint of any one of claims 50 to 52, comprising fibre with an average maturity ratio of ≥0.8.

54. The cotton lint of any one of claims 50 to 53, which has a dry basis moisture content of less than about 7% by weight.

55. The cotton lint of any one of claims 50 to 54, comprising fibre with one or more exogenous pigments that are present in the fibre at ≥40 DPA.

56. A method of producing yarn, the method comprising: (i) obtaining the cotton lint of any one of claims 50 to 55; and (ii) spinning the cotton lint to produce the yarn.

57. The method of claim 56, wherein step (i) further comprises harvesting the cotton lint from a cotton plant and/or ginning the cottonseed from the lint; and/or wherein step (ii) comprises one or more or all of carding, drawing, combing and roving, and spinning the cotton lint to produce the yarn.

58. The method of claim 56 or claim 57, wherein step (i) further comprises obtaining one or more additional fibres, and wherein step (ii) further comprises spinning the cotton lint and the one or more additional fibres to produce a blended yarn.

59. The method of claim 58, wherein the one or more additional fibres is selected from the group consisting of polyester, viscose, acrylic, wool and nylon.

60. The method of claim 58 or claim 59, wherein the blended yarn comprises at least about 30% cotton.

61. Yarn produced by the method of any one of claims 56 to 60.

62. Yarn comprising the cotton lint of any one of claims 50 to 55.

63. The yarn of claim 62, further comprising one or more additional fibres selected from the group consisting of polyester, viscose, acrylic, wool and nylon.

64. The yarn of claim 63, comprising at least about 30% cotton.

65. A textile comprising the yarn of any one of claims 61 to 64.

66. A method to produce a textile, the method comprising: (i) obtaining the yarn of any one of claims 61 to 65; and (ii) weaving or knitting the yarn into a fabric to produce the textile.
